SEQUENCE LISTING (16 sequences)

SEQ ID NO: 1 - DNA, 18 bp, Artificial sequence
  Synthetic oligonucleotide
tggagcaagtgcctcctc

SEQ ID NO: 2 - DNA, 21 bp, Artificial sequence
  Synthetic oligonucleotide
atattaccacttcgattggtc

SEQ ID NO: 3 - DNA, 21 bp, Artificial sequence
  Synthetic oligonucleotide
tggagctggaagtgcctcctc

SEQ ID NO: 4 - DNA, 21 bp, Artificial sequence
  Synthetic oligonucleotide
gtgatgggagagtccgataga

SEQ ID NO: 5 - DNA, 21 bp, Artificial sequence
  Synthetic oligonucleotide
gtcagctggggtcgtttcatc

SEQ ID NO: 6 - DNA, 21 bp, Artificial sequence
  Synthetic oligonucleotide
ctggttggcctgccctatata

SEQ ID NO: 7 - DNA, 21 bp, Artificial sequence
  Synthetic oligonucleotide
gtcagctggggtcgtttcatc

SEQ ID NO: 8 - DNA, 21 bp, Artificial sequence
  Synthetic oligonucleotide
gtgaaaggatatcccaataga

SEQ ID NO: 9 - DNA, 21 bp, Artificial sequence
  Synthetic oligonucleotide
gtagtcccggcctgcgtgcta

SEQ ID NO: 10 - DNA, 21 bp, Artificial sequence
  Synthetic oligonucleotide
gaccggttacacagcctcgcc

SEQ ID NO: 11 - DNA, 21 bp, Artificial sequence
  Synthetic oligonucleotide
gtagtcccggcctgcgtgcta

SEQ ID NO: 12 - DNA, 21 bp, Artificial sequence
  Synthetic oligonucleotide
tccttgcacaatgtcaccttt

SEQ ID NO: 13 - DNA, 21 bp, Artificial sequence
  Synthetic oligonucleotide
ccctgccgggatcgtgacccg

SEQ ID NO: 14 - DNA, 21 bp, Artificial sequence
  Synthetic oligonucleotide
tcgaagccgcggctgccaaag

SEQ ID NO: 15 - DNA, 25 bp, Artificial sequence
  Synthetic oligonucleotide
gccagctcaccatggatgatgatat

SEQ ID NO: 16 - DNA, 25 bp, Artificial sequence
  Synthetic oligonucleotide
gcttgctgatccacatctgctggaa

(19) United States
(12) Patent Application Publication
(10) Pub. No.: US 2006/0083711 A1
(43) Pub. Date: Apr. 20, 2006

(54) Methods and products related to the intracellular delivery of polysaccharides

(75) Inventors: David A. Berry, Brookline, MA (US); Daniel G. Anderson, Framingham, MA (US); David M. Lynn, Madison, WI (US); Ram Sasisekharan, Bedford, MA (US); Robert S. Langer, Newton, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Massachusetts Institute of Technology, Cambridge, MA (US)

(21) Appl. No.: 11/107,360

(22) Filed: Apr. 15, 2005

Related U.S. Application Data
(60) Provisional application No. 60/562,873, filed on Apr. 15, 2004.

(51) Int. Cl. A61K 31/785; A61K 31/737; A61K 31/727

(52) U.S. Cl. 424/78.12; 514/54; 514/56

(57) ABSTRACT

The invention relates, in part, to methods and compositions for the intracellular delivery of polysaccharides. In particular, the methods and compositions relate to the intracellular delivery of glycosaminoglycans, such as heparin. The invention in other aspects relates to the use of glycosaminoglycans for the treatment of proliferative disorders, such as cancer. The invention is still other aspects relates to improving cell viability. The invention also relates to the delivery of polysaccharides while avoiding unwanted effects of the polysaccharides. For example, heparin can be delivered while avoiding its anticoagulant effects.

### RELATED APPLICATIONS

This application claims priority under 35 U.S.C. §119 from U.S. provisional application Ser. No. 60/562,873, filed Apr. 15, 2004, the entire contents of which is herein incorporated by reference.

### GOVERNMENT SUPPORT

Aspects of the invention may have been made using funding from National Institutes of Health Grant numbers GM26698, CA52857, EB00244 and HL59966. Accordingly, the Government may have rights in the invention.

### FIELD OF THE INVENTION

The invention, in part, is directed to the intracellular delivery of polysaccharides, methods and compositions related thereto. In particular, the methods and compositions relate to the intracellular delivery of glycosaminoglycans, such as heparin. The invention in other aspects relates to the use of glycosaminoglycans for the treatment of proliferative disorders, such as cancer.

### BACKGROUND OF THE INVENTION

The role of glycosaminoglycans (GAGs) in influencing biological processes has been defined by their function in the extracellular matrix (ECM). Heparin/heparan sulfate-like glycosaminoglycans (HSGAGs) are linear polysaccharides found as the GAG component of heparan sulfate proteoglycans (HSPGs). Depending on the core protein, HSPGs are either free in the ECM or at the cell-ECM interface (Sasisekharan, R., et al., Nat. Rev. Cancer, 2:521-528 (2002)). Interactions between GAGs, such as HSGAGs, and other ECM components regulate important physiological and pathological processes including normal development, wound healing, and tumor progression (Perrimon, N., et. al, Nature, 404:725-728 (2000); Conrad, H. E., et al., San Diego: Academic Press (1998)).

The information rich nature of HSGAGs allows them to regulate such a wide variety of cell processes (Esko, J. D., et al., J. Clin. Invest., 108:169-173 (2001)). The HSGAG polysaccharide is composed of a disaccharide repeat unit consisting of a glucosamine linked to either an iduronic acid or a glucuronic acid. Potential 2-O sulfation on the uronic acid, 6-O and 3-O sulfation of the glucosamine, and an unmodified, acetylated or sulfated amine, lead to 48 potential disaccharide units that compose the 10-100-mer HSGAG chain (Perrimon, N., et. al, Nature, 404:725-728 (2000)). HSPGs are either at the cell-extracellular matrix (ECM) interface as with syndecans, or free in the ECM as with perlecans (Sasisekharan, R., et al., Nat. Rev. Cancer, 2:521-528 (2002)). In addition to the information content inherent to the polysaccharide chain (Blackhall, F. H., et al., Br. J. Cancer, 85:1094-1098 (2001), the tumorigenicity of a HSGAG chain is distinct whether it is free in the ECM or attached to an HSPG on the cell surface (Liu, D., et al., Proc. Natl. Acad. Sci USA, 99:568-573 (2002)).

In normal function, HSGAGs are brought into the cell in a controlled fashion. For example, HSGAGs bind to fibroblast growth factor (FGF) 2 and FGF receptor (FGFR) 1, forming a ternary complex that is internalized (Sperinde, G. V., et al., Biochemistry, 39:3788-3796 (2000)); Pellegrini, L., et al., Nature 407:1029-1034 (2000)). HSGAGs may facilitate the localization of the FGF-FGFR-HSGAG complex to the nucleus where it impacts cell function (Hsia, E., et al., J. Cell Biochem., 88:1214-1225 (2003)). Nonetheless the role of free HSGAGs within the cell has not been established.

### SUMMARY OF THE INVENTION

This invention provides, in part, methods and compositions related to the intracellular delivery of polysaccharides. In particular, the methods and compositions relate to the intracellular delivery of glycosaminoglycans, such as heparin. The invention in one aspect relates to the use of glycosaminoglycans for the treatment of proliferative disorders, such as cancer. In another aspect the invention relates to compositions that avoid unwanted properties of polysaccharides. In some embodiments the compositions provided comprise glycosaminoglycans that result in the intracellular delivery of the glycosaminoglycan.

In one aspect of the invention compositions are provided the comprise a glycosaminoglycan at a high dose. The high dose in one embodiment is one that results in a concentration of the administered glycosaminoglycan within at least one cell of greater than 1 mM. In other embodiments the high dose results in an intracellular concentration of the administered glycosaminoglycan equal to or greater than 5 mM, 10 mM, 20 mM, 50 mM, 75 mM, 100 mM, 125 mM, 140 mM or more. In another embodiment the high dose results in an intracellular concentration of the administered glycosaminoglycan of 150 mM.

The glycosaminoglycan in the compositions provided herein can be uncomplexed (not associated with another molecule) in the composition. The glycosaminoglycan can also be complexed with another molecule in the composition. Therefore, in another aspect of the invention compositions that comprise cationic polymer-glycosaminoglycan conjugates are provided. In some embodiments the compositions result in the reduction or avoidance of unwanted side effects normally associated with the administration of the glycosaminoglycan. In one embodiment the composition when administered results in reduced or no anticoagulation, anticoagulation being in some embodiments an unwanted side effect. Other examples of unwanted side effects include bleeding, heparin-induced thrombocytopenia, other heparin related side effects, etc. Therefore, in one aspect of the invention compositions and methods comprising a polysaccharide, such as a glycosaminoglycan, for intracellular delivery and that avoids or reduces at least one unwanted side effect are provided. Therefore, the compositions and methods provided results in none or less of the unwanted side effect as compared to the administration of the polysaccharide in ways other than those provided herein. The methods of administration provided result in the intracellular delivery of the polysaccharide.

According to one aspect of the invention, a composition is provided that comprises a cationic polymer and a polysaccharide wherein the polysaccharide is present in a therapeutically effective amount. In one embodiment it is the combination of the cationic polymer and polysaccharide that is in a therapeutically effective amount. In one embodiment the polysaccharide is a glycosaminoglycan. In another embodiment the glycosaminoglycan is not hyaluronic acid. In another embodiment the therapeutically effective amount is an intracellular therapeutically effective amount.

According to another aspect of the invention, a composition is provided that comprises a polysaccharide and a cationic polymer wherein the cationic polymer is not is not a protamine, a histone, a polyamino acid, or a polyamido amine.

According to still another aspect of the invention, a composition is provided that comprises a polysaccharide, a cationic polymer and a pharmaceutically acceptable carrier wherein more cationic polymer is present in the composition (w/w) than the polysaccharide.

According to yet another aspect of the invention, a composition is provided that comprises a poly(β-amino ester) and a polysaccharide.

According to still a further aspect of the invention, a composition is provided that comprises a cationic polymer, a polysaccharide and a targeting molecule. In certain embodiments the targeted cells are non-macrophage cells. In other embodiments of the invention, the targeted cells have increased endocytic rates. In yet other embodiments of the invention, the targeted cells with increased endocytic rates are cancer cells, such as epithelial cancer cells. In other embodiments the cancer with increased endocytic rates include adenocarcinomas (e.g., prostate and colon adenocarcinoma) and sarcomas (e.g., melanoma). In other embodiments the cells with increased endocytic rates are hyperplastic cells. In another embodiment the targeting molecule is a molecule that targets cancer cells. In still another embodiment the molecule that targets cancer cells is a molecule that binds a cancer antigen. In a further embodiment the molecule that binds a cancer antigen is an antibody, fragment of the antibody, binding peptide or a functional equivalent of the foregoing molecules.

In one embodiment the polysaccharides provided herein are therapeutic polysaccharides. In another embodiment the polysaccharide is an isolated polysaccharide. In still another embodiment the polysaccharide that is delivered intracellularly is both therapeutic and isolated. In one embodiment of the compositions and methods provided herein the polysaccharide is a glycosaminoglycan. In another embodiment the glycosaminoglycan is a heparin/heparin sulfate-like glycosaminoglycan (HSGAG). In another embodiment of the invention the glycosaminoglycan is heparin, biotechnologically prepared heparin, chemically modified heparin, synthetic heparin, heparan sulfate, enoxaparin, low molecular weight heparin (LMWH) or chondroitin sulfate. In still another embodiment the glycosaminoglycan is a chondroitin sulfate. In yet another embodiment the chondroitin sulfate is chondroitin sulfate A, chondroitin sulfate B, or chondroitin sulfate C. In still another embodiment the glycosaminoglycan is keratan sulfate. In yet another embodiment the glycosaminoglycan is dermatan sulfate. In still a further embodiment the glycosaminoglycan is highly sulfated, such as a highly sulfated HSGAG. In one embodiment the highly sulfated GAG, or HSGAG, has more than, on average, 1, 1.25, 1.5, 1.75, 2, 2.25, 2.5, 2.75, 3, 3.25, 3.5, or 3.75 sulfates per disaccharide. In another embodiment the highly sulfated GAG has on average 4 sulfates per disaccharide. In another embodiment the GAG has not been cleaved by a glycosaminoglycan-degrading enzyme. In still another embodiment the GAG has been cleaved by a glycosaminoglycan-degrading enzyme. In one embodiment the GAG is a HSGAG that has been cleaved with a heparinase. In one embodiment the HSGAG has been cleaved with a HSGAG-degrading enzyme. In another embodiment the HSGAG-degrading enzyme is a heparinase. In still another embodiment the heparinase is heparinase I and/or heparinase III. In another embodiment the glycosaminoglycan is one with a high charge density and/or high molecular weight. In one embodiment the glycosaminoglycan with a high molecular weight has a molecular weight greater than 3000 Da, 5000 Da, 7500 Da, 10000 Da or 15000 Da or more. An example of a glycosaminoglycan with high charge density is heparin and LMWH. In one embodiment the glycosaminoglycan with high charge density and high molecular weight is full length heparin. Other examples of glycosaminoglycans with high charge density and/or high molecular weight include full-length heparan sulfate, chondroitin sulfate, dermatan sulfate and hyaluronic acid. Therefore, in one embodiment the glycosaminoglycan is a full length glycosaminoglycan. In still another embodiment the glycosaminoglycan is not hyaluronic acid.

In one embodiment of the compositions and methods provided herein the cationic polymer is degradable. In another embodiment the polymer has low toxicity. In still another embodiment the polymer is biologically inert. In another embodiment the cationic polymer is one that promotes the uptake of a polysaccharide by a cell. In another embodiment the cationic polymer is a poly(β-amino ester). In still another embodiment the poly(β-amino ester) is A5, A8, A11, B6, B9, B11, B14, C4, C12, C32, D6, D94, E7, E14, E28, F20, F28, G5, C32-2, U28, U28-3, JJ28-3, D94-5, E28-3, U32, U32-2, JJ28, JJ32, JJ32-3, F28-6, F32 or F32-2.

In yet another embodiment of the compositions and methods provided herein there is two, three, four, five or more times (w/w) more cationic polymer than polysaccharide. In another embodiment the cationic polymer is complexed to the polysaccharide in a ratio of 2:1, 3:1, 4:1, 5:1, 10:1, 15:1, 20:1, 25:1, 30:1, 35:1, 40:1, 45:1, 50:1, or 60:1 or more.

In another embodiment of the compositions and methods provided herein the polysaccharide is in a therapeutically effective amount. In another embodiment the therapeutically effective amount is an amount effective to promote apoptosis. In another embodiment the therapeutically effective amount is an amount effective to inhibit cell proliferation. In yet another embodiment the therapeutically effective amount is an amount effective to treat a disease characterized by abnormal cell proliferation. In one embodiment the disease characterized by abnormal cell proliferation is cancer, Paget's disease, dermoid cysts, exuberant granulation, retinal detachment, cardiovascular conditions (e.g., restenosis (e.g., post angioplasty), atherosclerosis (e.g., from macrophage infiltrate), arteriosclerosis (e.g., from macrophage infiltrate), vasculidities (e.g., large-vessel vasculitis, such as embolic (clot, atheroemboli), giant-cell (temporal or cranial) arteritis (granulomatous), Takayasu arteritis (type I: aortic arch syndrome) types I-IV (granulomatous), syphilitic aortitis, non-luetic infectious aneurysms (salmonella, staph., enterococci), atherosclerotic aortic aneurysm and inflammatory abdominal aortic aneurysm; medium-sized vessel vasculitis, such as classical polyarteritis nodosa (CPN) (negative for MCLN; negative lungs), embolic vasculitis, Kawasaki disease, overlap syndromes of CPN, Churg-Strauss, and hypersensitivity vasculitis, Buerger's disease (thromboangiitis obliterans), MIVOD (mesenteric inflammatory veno-occlusive disease), isolated granulomatous phlebitis and idiopathic enterocolic lymphocytic phlebitis; small-vessel vasculitis, such as pauci-immune (few/no immune deposits) small-vessel vasculitis, Wegener's granulomatosis, microscopic polyangiitis, Churg-Strauss (allergic angiitis and granulomatosis) Syndrome (CSS), primary angiitis (granulomatous) of CNS (no skin lesions), drug-induced ANCA-positive vasculitis, isolated retinal vasculitis, arteritis & venulitis secondary), neurological disorders (e.g., Schwannosis, spinal cord injury, peripheral nerve injury), renal disease (e.g., polycystic kidney disease), muscular disorders (e.g., hereditary multiple exostoses, rheumatoid arthritis, and Osgood-Schlatter disease), infectious disease (e.g., human papaloma virus disease manifestations (warts), herpes simplex virus manifestations (ulcers), granulomatous disease (e.g., tuberculosis, sarcodoisis, Churg-Strauss Syndrome (allergic granulomatosis), Wegener's disease/granulomatosis, histiocytosis X), dermatalogical disorders (e.g., psoriasis, keloids), endocrine disorders (e.g., diabetic retinopathy), and metastatic cancer. In another embodiment the therapeutically effective amount is an amount effective to inhibit tumor angiogenesis. In yet another embodiment the therapeutically effective amount is an amount effective to inhibit aberrant neovascularization. In still another embodiment the therapeutically effective amount is an amount effective to treat cancer. In one embodiment the cancer is an adenocarcinoma. In another embodiment the cancer is a sarcoma. In still another embodiment the cancer is prostate cancer or colon cancer. In yet another embodiment the cancer is melanoma. In still a further embodiment the cancer is not lymphoma or leukemia. In yet another embodiment the therapeutically effective amount is an intracellular therapeutically effective amount. In one embodiment the intracellular therapeutically effective amount is where greater than 20%, 25%, 30%, 35%, 40%, 45%, 50%, 60,%, 75%, 90% or more of the cells contain the polysaccharide following administration.

In still another embodiment the compositions provided herein can further contain a targeting molecule. In one embodiment the targeting molecule targets a cancer cell. In yet another embodiment the compositions further comprise an additional therapeutic agent. In still another embodiment the additional therapeutic agent is an anti-cancer agent. In one embodiment the additional therapeutic agent is a glycosaminoglycan. The glycosaminoglycan can be the same or different than the polysaccharide for intracellular delivery in some embodiments of the compositions and methods provided herein. In still another embodiment the glycosaminoglycan is a HSGAG. In yet another embodiment the HSGAG is heparin. In still a further embodiment the additional therapeutic agent is a glycosaminoglycan-degrading enzyme. In yet a further embodiment the glycosaminoglycan-degrading enzyme is heparinase I and/or heparinase III. In another embodiment the additional therapeutic agent is protamine sulfate. In one embodiment where the compositions are to treat lymphoma the additional therapeutic agent is a heparinase or protamine sulfate or both. In another embodiment where the compositions are to treat melanoma, such as highly malignant melanoma, the additional therapeutic agent is FGF2. In still another embodiment the FGF2 is used as the additional therapeutic agent when the inhibition of cellular proliferation of B16F10 cells is desired.

In another aspect of the invention the compositions of the invention can be used to promote cell proliferation. Such compositions can be useful for cytological purposes, such as for growing up cells in culture. In one embodiment the compositions are used to grow Burkitt's lymphoma cells in culture.

In one embodiment of the compositions and methods provided herein the compositions further comprise a pharmaceutically acceptable carrier or physiologically acceptable carrier. In another embodiment the compositions further comprise sodium acetate or phosphate buffer saline (PBS). In still another embodiment the compositions provided herein are in a solution and have a physiological pH. In yet another embodiment the composition is in a vial or an ampoule. In still another embodiment the composition is a sterile composition.

In one embodiments the complexes of the cationic polymer and polysaccharide provided herein are positively or negatively charged. In one embodiment the complex of the polysaccharide and cationic polymer is positively charged. In another embodiment the complex has a positive zeta potential. In yet another embodiment the charge of the complex of the polysaccharide and cationic polymer is neutral. In still another embodiment the complex of the polysaccharide and cationic polymer has a diameter of less than 200 nm. In another embodiment the complex has a diameter of 10, 25, 50, 75, 100, 150 or 200 nm. In yet another embodiment the complex of the polysaccharide and cationic polymer has a diameter of greater than 200 nm. In another embodiment the complex has a diameter of 225, 250, 300, 350, 400, 500 nm or more. In still another embodiment the complex has a diameter greater than 250 nm.

The compositions can be administered to a subject in any way known to those of ordinary skill in the art. Therefore, in some aspects of the invention methods which comprise administering any of the compositions provided herein, or combinations thereof, to a subject are provided. In one embodiment the administration is local administration. In another embodiment the administration is intratumoral administration.

In one embodiment the subject has or is at risk of having a disease characterized by abnormal cell proliferation. In another embodiment the composition is administered in an amount effective to inhibit cell proliferation. In still another embodiment the composition is administered in an amount affective to promote apoptosis. The compositions administered can comprise the complexes of cationic polymer and polysaccharide, the polysaccharide itself, other therapeutic agents or some combination thereof. In another embodiment at least two compositions can be administered to a subject. In one embodiment the at least two compositions can be administered concurrently or at different times.

According to another aspect of the invention, a method for the intracellular delivery of a polysaccharide is provided. In one embodiment the method results in the delivery of the polysaccharide to the cytosol. In one embodiment the method results in the delivery of the polysaccharide in free form (uncomplexed, unconjugated) to the cell. In one embodiment the method comprises placing the polysaccharide in free form in contact with a cell. In another embodiment the polysaccharide is complexed with another molecule, and the complex is placed in contact with the cell. In one embodiment the other molecule is one that is rapidly degraded once in the cell and facilitates or allows for the polysaccharide's intracellular delivery. In another embodiment the polysaccharide is complexed to another molecule, wherein the other molecule is not a molecule normally associated with the polysaccharide in vivo. In one embodiment the polysaccharide is heparin and it is in complex with a molecule that is not a molecule that it normally binds in vivo (e.g., FGF2, FGFR1, etc.). In another embodiment the method comprises placing a glycosaminoglycan in free form in contact with a cell, wherein the glycosaminoglycan is in an intracellularly therapeutically effective amount.

In one aspect of the invention a method is provided for the intracellular delivery of a therapeutically effective amount of a polysaccharide that comprises administering the polysaccharide complexed to a cationic polymer to promote the uptake of the polysaccharide into a cell in a therapeutically effective amount.

According to another aspect of the invention a method is provided for administering a glycosaminoglycan intracellularly in an amount effective to promote apoptosis. In another aspect of the invention a method is provided for administering a glycosaminoglycan intracellularly in an amount effect to inhibit cell proliferation. In one embodiment of the invention the the glycosaminoglycan is complexed to a cationic polymer.

According to another aspect of the invention, a method is provided for treating a disease characterized by abnormal cell proliferation in a subject, that comprises administering a glycosaminoglycan intracellularly in an amount effective to treat the disease. In one embodiment the disease characterized by abnormal cell proliferation is cancer. In one embodiment the cancer is melanoma, hepatic adenocarcinoma, prostatic adenocarcinoma or osteosarcoma. In another embodiment the disease characterized by abnormal cell proliferation is Paget's disease, dermoid cysts, exuberant granulation, sarcoidosis and other granulomatous diseases, tuberculosis, diseases of abberrant inflammation (e.g., rheumatoid arthritis, lupus and spondyloarthropathies), scar formation and associated pathologies (e.g., keloids, spinal cord injury), skin infectious processes (e.g., warts, HPV infection) or retinal detachment.

According to another aspect of the invention, a method is provided for the intracellular delivery of a polysaccharide, that comprises administering the polysaccharide complexed to a poly(β-amino ester) in an amount effective to promote the uptake of the polysaccharide into a cell.

According to another aspect of the invention, a method is provided for the intracellular delivery of a polysaccharide, that comprises administering the polysaccharide complexed to a cationic polymer in an amount effective to promote the uptake of the polysaccharide into a cell, wherein the cationic polymer is not a protamine, a histone, or a polyamino acid.

According to another aspect of the invention, a method is provided for the intracellular delivery of a polysaccharide, that comprises administering the polysaccharide complexed to a cationic polymer in an amount effective to promote the uptake of the polysaccharide into a non-macrophage cell, wherein the polysaccharide is not present in excess of the cationic polymer.

In one embodiment the cells to which a polysaccharide is intracellularly delivered with the compositions and methods provided herein are non-SMC cells. In another embodiment the cells are not immunological cells. In still another embodiment the cells have increased endocytic rates. In yet another embodiment the cells are cancer cells. In still another embodiment the cells are epithelial cancer cells. In yet a further embodiment the cells are hyperplastic cells.

According to yet another aspect of the invention a method for promoting cell viability is provided. Cells in this aspect of the invention are contacted with a cationic polymer-heparin conjugate prior to freezing the cell in an amount effective to improve the cell's viability once subsequently thawed. Such cells can be any mammalian cell. In one embodiment, the cells are oocytes.

In still another aspect of the invention compositions containing heparin are provided. Such compositions can be used in methods of treating cancer and/or inhibiting or reducing cell proliferation. In other aspects of the invention compositions of GAG-degrading enzymes are provided that can also be used to reduce or inhibit cell proliferation. Such GAG-degrading enzymes include heparinase I and heparinase III. In yet other aspects of the invention cationic polymer-polysaccharide conjugates are provided which have been modified or degraded with GAG-degrading enzymes. Methods related to such compositions include administering the cationic polymer-modified or degraded polysaccharide conjugate. Such degraded polysaccharides include heparin degraded with heparinase I and/or heparinase III. Finally combinations of these molecules can be present in a composition in one aspect of the invention, and such compositions can be used in any of the methods described herein.

In one embodiment the methods provided are in vitro methods. In another embodiment the methods are in vivo methods.

In another aspect of the invention compositions comprising protamine sulfate and/or the heparinase enzymes provided herein are provided. The compositions can be used for any of the methods described. In one embodiment the compositions can be used to inhibit cancer cell proliferation, for example, lymphoma cell proliferation. In one embodiment the lymphoma cell proliferation is Burkitt's lymphoma cell proliferation. The compositions can be administered to a subject alone or in conjunction with another therapeutic composition, including those provided above. The different compositions can be administered concomitantly or at different times.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 illustrates that selected PAEs, polymers A5 and B6, enable internalization of heparin. SMCs were incubated with conjugates of fluorescein-labeled heparin and various polymers. Fluorescence microscopy images of polymers A5 (FIG. 1A) and B6 (FIG. 1B) are shown. Images are presented as an overlay of fluorescence onto light microscopy. Scale bars represent 10 μm. FIG. 1C shows the structure of the two polymers, polymers A5 and B6.

FIG. 2 shows that A5-heparin reduces B16-F10 growth. B16-F10 cells were treated with polymer-heparin conjugates alone (FIG. 2A) or with 5 ng/ml FGF2 (FIG. 2B). Data were normalized as percent reduction in whole cell count compared to untreated cells. B16-F10 cells were treated with A5-heparin at a 20:1 (w/w) ratio, or equivalent amounts of A5 alone (FIG. 2C). Whole cell count was converted to percent reduction compared to untreated cells. FIG. 2D shows the chemical structures of 4 polymers as labeled with notable cellular effects after conjugation to heparin.

FIG. 3 illustrates that A5-heparin affects cellular processes. B16-F10 cells were treated with A5-heparin conjugates at a 20:1 (w/w) ratio. Nuclear (FIG. 3A) and cytosolic (FIG. 3B) transcription factor levels were determined after incubation with conjugates for different time periods. Data were normalized to untreated cells and presented as the relative fold response compared to untreated. FIG. 3C shows the results from immunohistochemistry of B16-F10 cells after treatment with PBS, A5, A5-heparin conjugates, or heparin using antibodies specific to HS moieties.

FIG. 4 shows that heparin induced greater growth inhibition than other GAGs. The disaccharide composition of the various pools was determined by capillary electrophoresis after complete digestion by heparinases (FIG. 4A). Numbers represent the percentage of each given disaccharide. Not included was the undigestable 4-7 tetrasaccharide, which represents the deviation of the sum of each column from 100. B16-F10 cells were treated with GAGs (▪) and A5:GAG conjugates (▪; 20:1, w/w, 1 μg/ml heparin) (FIG. 4B). Hep, Eno, HA, LA, CS-A, and CS-C refer to heparin, enoxaparin, high activity LMWH, low activity LMWH, CS A and CS C. Data are expressed as whole cell number/100. Numbers represent the percent change in whole cell number for the A5:GAG conjugate compared to GAG alone.

FIG. 5 shows that A5-heparin exhibited cell selectivity. Cells were treated with A5-heparin (20:1, w/w; 1 μg/ml heparin) supplemented with PBS, FGF2, or sodium chlorate (FIG. 5A). Data are presented as percent of whole cell count compared to treatment without A5-heparin. Transfected BaF3 cells were not examined in the presence of chlorate due to the lack of cell surface GAGs. B16-BL6 and B16-F10 cells were treated with A5-fluorescein labeled heparin conjugates (20:1, w/w; 1 μg/ml heparin) (FIG. 5B). Cells were imaged by light microscopy, and fluorescein was visualized by fluorescence microscopy. Scale bars represent 10 μm.

FIG. 6 shows that A5-heparin induced cell death. B16-F10 cells were treated with A5-heparin conjugates at a 20:1 (w/w) ratio or equivalent concentrations of A5 or heparin alone. ³H-thymidine incorporation was measured by CPM over a range of heparin concentrations (FIG. 6A). 0 ng/ml represents untreated. Cytotoxicity measured by LDH assay was determined at 1 μg/ml heparin (FIG. 6B). Untx and Hep represent untreated and heparin respectively. Data are presented as percent of positive control, determined by (experimental point—negative control)/(positive control—negative control), where untreated is the negative control and Triton-X is the positive control. Apoptotic activity measured by caspase-3/-7 assays was determined at a heparin concentration of 1 μg/ml (FIG. 6C). Untx, Camp, and Hep represent untreated, camptothecin, and heparin respectively. Data are presented as percent of positive control, where untreated is the negative control and camptothecin is the positive control. *denotes p<0.05 compared to the negative control.

FIG. 7 shows that A5-heparin induced spermine incorporation at 6 hours. Incorporation of ¹⁴C-spermine was measured over time after treatment of SMCs (FIG. 7A), B16-BL6 cells (FIG. 7B), and B16-F10 with A5:heparin conjugates (20:1, w/w; 1 μg/ml) (FIG. 7C). S and D denote 5 μM spermine and 5 mM DFMO respectively. Numbers along the x-axis reflect conjugate incubation time. Data are presented as CPM.

FIG. 8 illustrates the absolute growth inhibition with internalized heparin. The heparin was conjugated to a number of polymers at the following ratios 10:1, 20:1, 30:1, 40:1, 50:1 and 60:1 (polymer:heparin).

FIG. 9 shows that heparin inhibited PC-3 growth by inhibiting FGF2. PC-3 cells were treated with various amounts of heparin or heparan sulfate (HS) (FIG. 9A). Heparin was pretreated with PBS, heparinase I (hepI) or hepIII prior to application to PC-3 cells (FIG. 9B). *denotes p<0.05 compared to untreated heparin. PC-3 cells were treated with various amounts of fibroblast growth factor (FGF) 2 (FIG. 9C). PC-3 cells were treated with FGF-2 and various amounts of heparin (FIG. 9D).

FIG. 10 shows that heparin mediated inhibition of PC-3 growth is dependent on FGF2 activity. RT-PCR was performed on PC-3 cells for actin (ACT) as well as FGFR isoforms (1b, 1c, 2b, 2c, 3b, 3c, and 4) (FIG. 10A). PC-3 cells were treated with antibodies to FGF2, FGF receptor (FGFR) 1, and FGFR3 in the presence or absence of heparin (FIG. 10B). Data are presented as percent inhibition in the presence of heparin compared to antibody alone.

FIG. 11 shows that heparin inhibited the FGF2 signaling cascade. PC-3 cells were treated with PBS, heparin or FGF2. ELISAs were performed on cell lysates. Concentrations of Erk1 (FIG. 11A), Erk2 (FIG. 11B), and p-Erk1/2 (FIG. 11C) were determined using rabbit anti-human primary antibodies and HRP conjugated goat anti-rabbit seconday antibodies. Data are normalized as concentration relative to PBS treated cells. *denotes p<0.05 compared to untreated.

FIG. 12 provides the results of PC-3 cells treated with LY294002 (LY), PD98059 (PD), or SB203580 (SB), or U0126 (U) in the presence or absence of heparin. Data are presented as percent inhibition in the presence of heparin compared to kinase inhibitor in the absence of heparin. *denotes p<0.05 versus the percent inhibition induced by heparin in the presence of PBS.

FIG. 13 shows that A5-heparin conjugates induced apoptotic cell death in B16-F10 cells. FIG. 13A provides the chemical structure of A5. B16-F10 cells were incubated with conjugates of A5 and fluorescein-labeled heparin for 6 hours and visualized by fluorescence microscopy (FIG. 13B). The scale bar represents 10 μm. B16-F10 cells were treated with PBS, 20 μg/ml A5, 1 μg/ml heparin, or A5-heparin conjugates (20:1, w/w, 1 μg/ml heparin) (FIG. 13C). Whole cell number was determined after three days and converted to a percent reduction in whole cell number compared to cells treated with PBS. *denotes p<0.05 compared to PBS. B16-F10 cells were treated with A5-heparin conjugates (20:1, w/w) or an equivalent amount of A5 alone (FIG. 13D). Whole cell data determined after three days were normalized as the percent reduction compared to cells treated with PBS. Caspase-3/-7 assays, used as a measure of apoptosis, were performed under similar conditions as proliferation assays (FIG. 13E). Camp and Hep represent camptothecin and heparin, respectively. Data are presented as percent of positive control, where PBS is the negative control and camptothecin is the positive control. *denotes p<0.05 compared to PBS.

FIG. 14 shows that A5-heparin was internalized in Daudi cells and promoted concentration dependent proliferation. Daudi cells were treated with PBS, 20 μg/ml A5, 1 μg/ml heparin or A5-heparin (20:1 ratio, w/w, 1 μg/ml heparin) (FIG. 14A). Daudi cells were treated with A5-heparin (20:1, w/w) over a range of heparin concentrations (FIG. 14B). Whole cell number was determined after three days. Data are expressed as percent growth compared to PBS treatment. Daudi cells were incubated with conjugates of A5 and fluorescein-labeled heparin for 24 hours and visualized with fluorescence microscopy (FIG. 14C) or light microscopy (FIG. 14D). Scale bars represent 10 μm.

FIG. 15 shows that A5-heparin promotes cell proliferation and apoptosis. Daudi cells were treated with A5-heparin (20:1, w/w, 1 μg/ml heparin) over a range of heparin concentrations. Proliferation was measured using a MTS assay (FIG. 15A). Data were normalized as a percent change from the untreated condition. Cytotoxicity was measured using a LDH assay (FIG. 15B). Data were converted to a percentage of the change induced by Triton-X, the positive control, relative to PBS treatment, the negative control. Apoptosis was measured using a caspase-3/-7 assay (FIG. 15C). Data were converted to a percentage of the change induced by camptothecin, the positive control, relative to PBS treatment, the negative control.

FIG. 16 shows that polymer-1 heparin promoted PI3K- and Erk/Mek-dependent proliferation requiring cell surface HSGAGs. Daudi cells were treated with PBS or A5-heparin (20:1, w/w, 1 μg/ml heparin) supplemented with PBS, 50 μM LY294002 (LY), 20 μM PD98059 (PD), or 1 μM SB203580 (SB) (FIG. 16A). Whole cell number was determined after 72 hrs, and converted to the percent of proliferation from PBS treatment. *denotes p<0.05 compared to PBS treatment. Heparin pretreated with PBS, hepI, or hepIII, and conjugated with A5 is shown in (FIG. 16B). Data are presented as percent increase in whole cell number compared to treatment with PBS alone. PBS, hepI, and hepIII refer to the treatment of heparin prior to conjugation with A5. *denotes p<0.05 compared to Daudi treated with PBS. **denotes p<0.05 compared to A5 conjugated with PBS treated heparin.

FIG. 17 illustrates that HSGAGs offer a viable target to influence BL proliferation. Daudi cells pretreated with PBS, 10 ng/ml FGF2, or 50 mM sodium chlorate were supplemented with PBS, heparin, or A5-heparin (20:1, w/w, 1 μg/ml heparin) (FIG. 17A). Whole cell number was determined after 72 hours, and normalized as the percent of the whole cell number after PBS treatment. *denotes p<0.05 compared to the corresponding treatment (PBS, heparin, or A5-heparin) pretreated with PBS. Daudi cells in propagation media were treated with P.BS, A5, heparin, or A5-heparin (20:1, w/w, 1 μg/ml heparin) (FIG. 17B). Data are presented as percent increase in whole cell number compared to treatment with PBS alone. *denotes p<0.05 compared to Daudi treated with PBS alone.

FIG. 18 shows that protamine sulfate inhibited Daudi proliferation at high concentrations. Daudi cells in propagation media were treated with protamine sulfate over a range of concentrations, and whole cell number was determined after three days. Data are expressed as the percentage of the whole cell number after PBS treatment. *denotes p<0.05 compared to PBS treatment.

FIG. 19 shows that hepI effectively inhibited Daudi proliferation in serum. Daudi cells in propagation media (10% FBS) were treated with various concentrations of hepI (FIG. 19A) or hepIII (FIG. 19B), and incubated for 24, 48, 72 hours. Cells were counted after the incubation, and cell number was normalized as the percent reduction in whole cell number compared to PBS treatment.

FIG. 20 shows that polymer heparin has a similar effect on the inhibition of tumor growth in vivo and in vitro: treated side (FIG. 20A), untreated side (FIG. 20B), weight (FIG. 20C). The figure shows the effects on the tumor over time.

FIG. 21 shows that heparin inhibits PC-3 proliferation. PC-3 cells were treated with various concentrations of heparin or HS (FIG. 21A). Heparin pretreated with PBS, hepI, or hepIII was applied to PC-3 cells (FIG. 21B). Whole cell number was determined after a 72 hour incubation. Data were normalized to the final whole cell number of PBS-treated cells and presented as the percent reduction in final whole cell number.

FIG. 22 shows that exogenous heparin inhibits FGF2-mediated proliferation. RT-PCR was performed on PC-3 cells for actin (ACT) as well as FGFR isoforms (1b, 1c, 2b, 2c, 3b, 3c, and 4) (FIG. 22A). Various concentrations of FGF2 were administered to PC-3 cells (FIG. 22B). PC-3 cells were treated with 100 ng/ml FGF2 and various concentrations of heparin (FIG. 22C). Whole cell number was determined after a 72 hour incubation. Data were normalized to the final whole cell number of PBS-treated cells and presented as the percent increase in final whole cell number.

FIG. 23 shows that heparin inhibits PC-3 tumor growth in vivo. PC-3 cells were injected into mouse flanks and allowed to grow to ˜50 mm³ tumors. Tumors were treated with daily injections of NaOAc (the negative control), 5 ng, 50 ng, or 500 ng heparin, and tumor size was measured over eight days (FIG. 23A). *denotes p>0.05 for tumors treated with 500 ng heparin compared to NaOAc. † denotes p>0.05 for tumors treated with 50 ng heparin compared to NaOAc. § denotes p>0.05 for tumors treated with 5 ng heparin compared to NaOAc. Tumors were injected only on day 0, with NaOAc (the negative control), 500 ng, or 400 μg heparin (FIG. 23B). *denotes p<0.05 for heparin treatment compared to the NaOAc control. Tumor volume was measured over eight days. Measurements on day 8 are presented. Data are presented as tumor size from day x/tumor size from day 0. A value of 1 denotes no growth. *denotes p<0.05 compared to NaOAc.

FIG. 24 shows that internalized heparin inhibits PC-3 proliferation more efficiently than heparin alone. PAE-heparin conjugates were formed at 60:1 (w/w) for C32, 60:1 (w/w) for U28, and 10:1 (w/w) for F32 with 1 μg/ml heparin, and used to treat PC-3 cells (FIG. 24A). F32 was conjugated at 10:1 (w/w) with heparin, and added to PC-3 cells at various heparin concentrations (FIG. 24B). Whole cell number was determined after a 72 hour incubation. Data were normalized to the final whole cell number of PBS-treated cells and presented as the percent reduction in final whole cell number.

FIG. 25 shows that internalized heparin prevents PC-3 tumor growth. PC-3 cells were injected into mouse flanks and allowed to grow to ˜50 mm³ tumors. Tumors were injected once, with NaOAc (the negative control), 5 μg, 50 μg, or 500 μg heparin, or the equivalent amounts of heparin conjugated to F32 at a 10:1 polymer:heparin ratio (w/w). Tumor volume was measured over eight days. Measurements on day 8 are presented. Data are presented as tumor size from day x/tumor size from day 0. A value of 1 denotes no growth. *denotes p<0.05 compared to NaOAc. † denotes p<0.05 for heparin compared to polymer-heparin conjugates.

FIG. 26 provides examples of PAE components.
*Everywhere herein Polymer 1 is A5.

### DETAILED DESCRIPTION

The invention relates in part to the discovery that glycosaminoglycans that are delivered intracellularly can modulate cell proliferation. For example, and as provided below in the Examples, it was found that heparin when administered and taken up by cancer cells caused the inhibition of cancer cell proliferation. High doses of internalized heparin were also found to inhibit tumor growth in vivo. Therefore, glycosaminoglycans can be administered in high doses to inhibit tumor cell proliferation. Compositions are provided the comprise a glycosaminoglycan at a high dose. The high dose, for example, results in an intracellular concentration of the administered glycosaminoglycan of greater than 1 mM. In other embodiments the high dose results in an intracellular concentration of the administered glycosaminoglycan equal to or greater than 5 mM, 10 mM, 20 mM, 50 mM, 75 mM, 100 mM, 125 mM, or 140 mM. In another embodiment the high dose results in an intracellular concentration of the administered glycosaminoglycan of 150 mM.

The invention also relates in part to the discovery that cationic polymers enable the intracellular delivery of polysaccharides. As provided below in the Examples, cationic polymers, such as poly(β-amino ester)s (PAEs), were successfully used for the intracellular delivery of a number of glycosaminoglycans. The delivery of these glycosaminoglycans were, in turn, found to control cell proliferation. In addition, subsequent studies showed that A5-heparin conjugate inhibited cell growth through the induction of apoptosis.

Glycosaminoglycans, such as the anionic biopolymers heparin/heparan sulfate-like glycosaminoglycans (HSGAGs), are involved in diverse cellular processes in the extracellular matrix (ECM). Heparin is a prototypical HSGAG that is more negatively charged than other HSGAGs due to the high quantity of sulfate groups found on the composite disaccharides. The biological effect of HSGAGs is dependent on their disaccharide content and physiological location within the ECM. Correspondingly, the relative biological location of the HSGAG chain and the HSPG core protein influences function. HSGAGs are normally brought into cells during membrane transcytosis and growth factor signaling while protein bound. The impact of free HSGAGs within the cell using heparin as a model HSGAG has now been determined.

Poly(β-amino ester)s (PAEs) are a class of cationic polymers that bind to DNA and enable its internalization by endocytosis [10, 11]. A library of polymers, poly(β-amino ester)s, which interact with DNA through a charge-mediated mechanism, and enable its internalization, were used to investigate their binding to various glycosaminoglycans, the uptake of polymer-glycosaminoglycan conjugates into cells and the resulting effects. It was found that all water soluble polymers tested bound heparin, and a subset of the polymers that can internalize DNA, were sufficiently cationic to internalize the more anionic heparin. It was also found that a number of polymer-glycosaminoglycan conjugates had growth inhibiting effects. For instance, the A5-heparin conjugate reduced murine melanoma cell growth 73%, while F32-2-heparin conjugates inhibited growth 84.5%. In addition, the impact of free heparin was also determined. It was found that the uptake of A5 polymer-heparin conjugate into cells induced apoptotic cell death, limited primarily by the rate at which cells internalized the conjugate. Cationic polymers, therefore, that bind polysaccharides, such as heparin, can sufficiently promote polysaccharide uptake into cells. Further, glycosaminoglycans provide a mechanism to induce apoptosis of cancer cells, and their internalization, for example, by cationic polymers, such as poly(β-amino ester)s, offers an approach to induce cancer cell death.

The invention, therefore, relates, in part, to compositions of cationic polymer and polysaccharide. As used herein, the term “cationic polymer” refers to any polymer or a portion thereof with a net positive charge. The cationic polymers include poly(β-amino ester)s, such as those described herein, including A5, A8, A11, B6, B9, B11, B14, C4, C12, C32, D6, D94, E7, E14, E28, F20, F28, G5, C32-2, U28, U28-3, JJ28-3, D94-5, E28-3, U32, U32-2, JJ28, JJ32, JJ32-3, F28-6, F32 and F32-2. Typically, these polymers have one or more tertiary amines in the backbone of the polymer. Poly(β-amino ester) polymers may also be copolymers in which one of the components is a poly(β-amino ester). These polymers can be prepared, for example, by condensing bis (secondary amines) or primary amines with bis (acrylate esters). Poly(β-amino ester)s and methods of their production are also described in U.S. Patent Application publication 20020131951 published Sep. 19, 2002. The structures for a library of 94 poly(β-amino ester)s as well a methodology for their synthesis can be found in Anderson et al., “Semi-Automated Synthesis and Screening of a Large Library of Degradable Cationic Polymers for Gene Delivery”, Angew. Chem. Int. Ed. 2003, 42, 3153-3158. A library of 140 poly(β-amino ester)s is described in Lynn et al., “Accelerated Discovery of Synthetic Transfection Vectors: Parallel Synthesis and Screening of a Degradable Polymer Library”, J. Am. Chem. Soc. 2001, 123, 8155-8156.

Cationic polymers can also include natural cationic polymers, such as proteins and peptides or synthetic cationic polymers, such as poly(ethylene imine) (PEI). The natural cationic polymer in one embodiment, however, is a polymer that is not usually associated with the polysaccharide in vivo. In some embodiments, the cationic polymer is degradable. Degradable cationic polymers can contain both chargeable amino groups, to allow for ionic interaction with the negatively charged polysaccharides, and a degradable region, such as a hydrolyzable ester linkage. Examples of these include poly(alpha-(4-aminobutyl)-L-glycolic acid), network poly(amino ester), polyethylene imine, polylysine, polyarginine and poly (β-amino ester)s as provided above. In other embodiments the cationic polymer is rapidly degradable. “Rapidly degradable” as used herein refers to the relatively short amount of time required to break down the cationic polymer into its constituent parts. The speed of degradability can be assessed by comparison, for instance, to polylysine. In some embodiments, a rapidly degradable polymer is one that is degraded faster than polylysine under the same conditions. The degradation may be by enzymatic or hydrolytic degradation. In yet other embodiments, the cationic polymer is a cationic polymer as defined above but is not a protamine, a histone, a polyamino acid, or a polyamido amine. In still other embodiments the cationic polymers as provided herein are not polyornithine or polylysine. Preferably, the cationic polymers employed in the compositions provided, particularly, those used for the intracellular delivery of polysaccharide in a subject, are cationic polymers with low toxicity. A “cationic polymer with low toxicity” is one that is less toxic than polylysine when compared in the same amount under the same conditions. In some instances a cationic polymer with toxicity greater than or equal to polylysine may be desired. In still another embodiment the cationic polymer is biologically inert. As used herein a “biologically inert” cationic polymer is one that when administered alone to a subject or placed in contact with one or more cells the cationic polymer itself does not affect or significantly affect any biological processes. In one embodiment the cationic polymer facilitates and/or does not substantially inhibit the polysaccharide's intracellular delivery.

The polysaccharides for use in the compositions provided include any molecule which contains two or more consecutively linked monosaccharides. Polysaccharides may include those that are isolated from plant, animal and microbial sources. The term “polysaccharide” as used herein, therefore, include mucins, alginates, pectins, fucoidans, carrageenans, chitin, pentosan, dextran sulfate, laminarin, fucans, glucans, calcium spirulan, xylan, amylose, cellulose, curdlan, trehalose, glycans, mannitol, galactose, sucrose and D-galactan. The polysaccharides also may include glycosaminoglycans, a family of complex polysaccharides that include dermatan sulfate (DS), chondroitin sulfate (CS), heparin, heparan sulfate, keratan sulfate, and hyaluronic acid. The term “polysaccharide”, therefore, also refers to highly sulfated glycosaminoglycans. These glycosaminoglycans can have a high molecular weight and/or high charge density. In one embodiment the glycosaminoglycan with a high molecule weight and/or high charge density is a full length glycosaminoglycan, such as heparin, heparan sulfate, chondroitin sulfate, dermatan sulfate or hyaluronic acid. Other example of glycosaminoglycans include those with a molecular weight greater than 3000 Da, 5000 Da, 7500 Da, 10000 Da, or 15000 Da. Still other examples of glycosaminoglycans include heparin/heparin sulfate-like glycosaminoglycans, biotechnologically prepared heparin, chemically modified heparin, synthetic heparin, heparinoids, enoxaparin, low molecular weight heparin (LMWH), or specific kinds of chondroitin sulfate, such as chondroitin sulfate A, chondroitin sulfate B or chondroitin sulfate C. In one embodiment the polysaccharide is not hyaluronic acid. Polysaccharides, in some embodiments, may also include heparin-like polyanions which are similar to heparin and are naturally occurring or synthetic. Such heparin-like polyanions include poly(vinyl sulfate) and poly(anethole sulfonate). The glycosaminoglycans also include highly sulfated glycosaminoglycans, such as highly sulfated HSGAGs. The highly sulfated GAGs can contain 1, 1.25, 1.5, 1.75, 2, 2.25, 2.5, 2.75, 3, 3.25, 3.5, 3.75 or more, etc. sulfates per disaccharide. The highly sulfated GAGs include oversulfated chondroitin sulfate and oversulfated dermatan sulfate. As used herein, the term heparin is meant to encompass any molecules which are functional equivalents to heparin. Likewise, naming of a specific type of polysaccharide is intended to include the functional equivalents of that polysaccharide. Polysaccharides that are naturally derived or are synthetic are also intended to be included.

The polysaccharides can also be modified versions of the polysaccharides provided herein. These “modified polysaccharides” can be modified by depolymerization, phosphorylation, sulfonation, regioselective sulfonation and/or desulfonation. In particular, in some embodiments the modified polysaccharides are sulfated versions of a polysaccharide provided herein. Examples of such sulfated polysaccharides include sulfated D-galactan, sulfated α-(1-3)-D-glucan, laminarin sulfate, natural sulfated fucans, etc. The polysaccharides for use in the compositions and methods described herein, therefore, include polysaccharides that have been modified with polysaccharide degrading enzymes. “Polysaccharide degrading enzymes” are enzymes that cleave, degrade or somehow modify a polysaccharide when placed in contact with the polysaccharide. Polysaccharide degrading enzymes include but are not limited to chondroitinases (e.g. chondroitinase AC, chondroitinase B), hyaluronate lyase, heparinases (e.g., heparinase-I, heparinase-II, heparinase-III), keratanase, D-glucuronidase and L-iduronidase, 2-O sulfatase, 3-O sulfatase, 6-O sulfatase, C5-epimerase, sulfotransferases, such as 2-O sulfotransferase, 3-O sulfotransferase, 6-O sulfotransferase, and N-sulfotransferase (NDST) modified versions of these enzymes, variants and functionally active fragments thereof. Polysaccharide-degrading enzymes, therefore, also include “glycosaminoglycan-degrading enzymes”, which are enzymes that cleave, degrade or somehow modify a glycosaminoglycan when placed in contact with the glycosaminoglycan.

The compositions of cationic polymer and polysaccharide are those whereby the cationic polymer and polysaccharide are conjugated, or in other words, form a complex. The complexes formed can be created through any means that are known in the art. The complexes of cationic polymer and polysaccharide can be formed from electrostatic interactions between the cationic polymer and polysaccharide. Generally, the electrostatic interactions will be between the positive charges present on the cationic polymer and the negative charges of the polysaccharide, particularly when the polysaccharide is an anionic polysaccharide, such as heparin. The cationic polymer-polysaccharide complexes, however, do not have to be formed from electrostatic interactions. One of ordinary skill in the art can envision ways of conjugating the molecules through the use of covalent bonds or linker molecules. The covalent bonds or linker molecules can be, in some embodiments, degradable. The covalent bonds or linker molecules, such as mono- and hetero-bifunctional linkers, employ routine chemistry, which is well known to those skilled in the art.

Specific examples of covalent bonds include those wherein bifunctional cross-linker molecules are used. The cross-linker molecules may be homo-bifunctional or hetero-bifunctional, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl) suberate, bis[2-(succinimidooxycarbonyloxy)ethyl]sulfone, disuccinimidyl suberate, disuccinimidyl tartarate, dimethyl adipimate•2 HCl, dimethyl pimelimidate•2 HCl, dimethyl suberimidate•2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide•2 HCl, 4-(4-N-maleimidophenyl)-butyric acid hydrazide•2 HCl, and 3-[2-pyridyldithio]propionyl hydrazide. The cross-linkers are bis-[β-4-azidosalicylamido)ethyl]disulfide and glutaraldehyde. Additionally, amine or thiol groups may be added to the molecules of the invention so as to provide a point of attachment for a bifunctional cross-linker molecule.

The complexes formed of the cationic polymer and polysaccharide can be neutral. In other embodiments, the complexes are not neutral but are negatively or positively charged. The complexes include those with a positive zeta potential. The charge of the cationic polymer-polysaccharide complexes is determined through the charge densities of the individual molecules as well as the amount of cationic polymer relative to the amount of polysaccharide (w/w) present to form the complex. In some embodiments the complexes have a net positive zeta potential. In other embodiments the complexes have a net negative zeta potential. Preferably, the complexes will contain more cationic polymer (w/w) than polysaccharide. In some embodiments, the complexes will be made up of 2, 3, 4, 5, 6, 7, 8, 9, 10, 15, 20, 25, 30, 35, 40, 50, 60, 75 or more times (w/w) more cationic polymer than polysaccharide. The complexes of the compositions provided herein may in some embodiments have a ratio of cationic polymer to polysaccharide (w/w) of 2:1, 3:1, 4:1, 5:1, 10:1, 20:1, 30:1, 40:1, 50:1 or 60:1.

The complexes of cationic polymer and polysaccharides provided herein also include complexes that are internalized rapidly and/or keep the polysaccharide in the cell for a period of time. Methods for analyzing the internalization of the polysaccharide into a cell are known in the art and are also provided below in the Examples. As used herein to be “internalized rapidly” means that the polymer-polysaccharide conjugate is internalized within 1, 2, 3, 4, 5 or 6 hours. Still other complexes that are rapidly internalized are those that are internalized within less than 24 hours. Preferably, the complexes keep the polysaccharide, once internalized, in a cell for more than 6, 7, 8, 9, 10, 11, 12, 13, 14, 15, 16, 17, 18, 19, 20, 21, 22, 23, or 24 or more hours. Also preferred are complexes that cause the polysaccharide to be delivered to the cytosol or other non-reticulo locations.

Additionally, in some embodiments the complexes provided herein have an “effective diameter”. As used herein the “effective diameter” of the complexes is one that allows for the internalization of a particular polysaccharide. In some embodiments, the effective diameter is less than 200 nm. In some embodiments, the effective diameter is 10 nm, 20 nm, 30 nm, 40 nm, 50 nm, 60 nm, 75 nm, 100 nm, 150 nm, 175 nm or less. However, in other embodiments the effective diameter is greater than 200 nm. Particularly, in some embodiments, the effective diameter is 210 nm, 220 nm, 230 nm, 240 nm, 250 nm, 275 nm, 300 nm, 400 nm, 500 nm or more.

In some embodiments the cationic polymers and/or polysaccharides are in a substantially pure form. As used herein, with respect to these molecules, described herein, the term “substantially pure” means that the molecules of the invention are essentially free of other substances with which they may be found in nature or in vivo systems to an extent practical and appropriate for their intended use. In particular, the molecule is sufficiently free from other biological constituents of their hosts cells so as to be useful in, for example, producing pharmaceutical preparations. Because the molecules of the invention may be admixed with a pharmaceutically acceptable carrier in a pharmaceutical preparation, the molecule may comprise only a small percentage by weight of the preparation. The molecule is nonetheless substantially pure in that it has been substantially separated from the substances with which it may be associated in living systems. Polysaccharides can be isolated from biological samples or can be synthesized using standard chemical synthetic methods. Cationic polymers likewise can be isolated from biological samples or can be synthesized using standard chemical synthetic methods. Some cationic polymers, such as proteins and peptides, can also be expressed recombinantly in a variety of prokaryotic and eukaryotic expression systems by constructing an expression vector appropriate to the expression system, introducing the expression vector into the expression system, and isolating the recombinantly expressed protein.

As used herein with respect to the molecules provided herein, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e., isolated from other proteins.

In some of the compositions provided herein, the polysaccharide is present in a therapeutically effective amount. As used herein, the polysaccharide can have any of a number of therapeutic activities. For instance, in some embodiments the polysaccharide is in a therapeutically effective amount to promote apoptosis. The term “therapeutically effective amount” also includes an amount of the polysaccharide that inhibits cell growth. The therapeutically effective amount is, therefore, in some embodiments, such an amount that would be useful to inhibit or retard cell proliferation. Therapeutically effective amount, therefore, also includes an amount effective to treat a disease characterized by abnormal cell proliferation. In some embodiments the therapeutically effective amount of the polysaccharide is sufficient to neutralize FGF2 mediated proliferation. This amount can be, for instance, an amount of the polysaccharide that is equal to or greater than the amount of FGF2 in a sample in vitro or in a specific location in a subject. In one embodiment a therapeutically effective amount is an intracellular therapeutically effective amount. This term refers to the percentage of cells, to which a polysaccharide in complexed or uncomplexed form has been placed in contact with, that contains (within the cell) the administered polysaccharide. In one embodiment the intracellular therapeutically effective amount is when greater than 20%, 25%, 30%, 35%, 40%, 45%, 50%, 55%, 60%, 65%, 70%, 75%, 80%, 85%, 90%, 95% or more of the cells contacted with the complexed or uncomplexed polysaccharide contain the polysaccharide. As one non-limiting example the intracellular therapeutically effective amount is when greater than 20%, 25%, 50%, 75%, 90%, 95% or more of the cells of a tumor contain the administered polysaccharide.

Compositions are also provided that comprise a polysaccharide in uncomplexed form (i.e., not complexed to a cationic polymer and/or not associated with any molecule) and in an intracellular therapeutically effective amount. The polysaccharide can be any of the polysaccharides described herein.

The compositions provided can also be a solution. In one embodiment the solution has a physiological pH. In another embodiment the composition can further contain a pharmaceutically acceptable or physiologically acceptable carrier. In still another embodiment the composition can contain sodium acetate and/or PBS.

Based on the demonstrated activity of a number of glycosaminoglycans described herein, the invention relates, in part, to a method for the intracellular delivery of a polysaccharide. In some embodiments the polysaccharide is in free form (i.e., uncomplexed) is placed in contact with one or more cells in an intracellular therapeutically effective amount. In other embodiments the polysaccharide in a liposome, microsphere or nanoparticle is placed in contact with one or more cells and delivered intracellularly. In one embodiment the polysaccharide is complexed with a molecule that is not a liposome. In another embodiment the polysaccharide is complexed with another molecule covalently. In another embodiment the polysaccharide is complexed with another molecule non-covalently. In other embodiments the polysaccharide is complexed to a cationic polymer, such as a poly(β-amino ester), in an amount effective to promote the uptake of the polysaccharide into one or more cells. In some embodiment where the polysaccharide is complexed to another molecule, such as those described above, the other molecule is one that facilitates or does not hinder the internalization of heparin and is degraded such that heparin is in free form (uncomplexed) at some point after administration within the cell. The polysaccharide in some of the methods provided is not present in excess of the cationic polymer. In some embodiments the cationic polymer is not a protamine, a histone, or a polyamino acid. In other embodiments the intracellular delivery of a polysaccharide is into a non-immunological cell or a non-macrophage cell. In still other embodiments the cells are not smooth muscle cells. In yet other embodiments the cells to which a polysaccharide is delivered into are cells with a high endocytic rate. Such cells include cancer cells, epithelial cancer cells or hyperplastic cells.

In one aspect of the invention a method for promoting cell viability is provided using the compositions provided herein. In one embodiment the composition contains one a cationic polymer-polysaccharide conjugate described herein. In some embodiments the conjugate for promoting cell viability is a cationic polymer-monosaccharide conjugate. Generally, the cells can be contacted with the compositions provided in order to deliver the polysaccharide into the cells prior to freezing in an amount effective to promote cell viability when thawed. In some embodiments, the method provides an after-thaw cell viability of 10%, 20%, 30%, 40%, 50%, 60%, 70%, 80% or 90%. The mono- or polysaccharides for use in this method can be any saccharide that causes improved after-thaw cell viability as opposed to the after-thaw viability of cells that are frozen without the delivery of intracellular saccharides. Such saccharides may include mono-, di-, tri-, and polysaccharides. In some embodiments, the saccharide is trehalose. In still other embodiment the saccharide is a GAG, such as heparin. The cells of this aspect of the invention can be any mammalian cell. In one embodiment the cells are oocytes. The cells can be contacted with the compositions via injection with a needle in one embodiment.

Methods for a number of therapeutic purposes are provided herein. These methods include methods for promoting apoptosis in a subject, methods for inhibiting cell growth and methods for mediating cell proliferation, inluding FGF2 mediated cell proliferation. In these methods, a polysaccharide such as a glycosaminoglycan is administered intracellularly in an amount effective to achieve the therapeutic endpoint. Methods, therefore, are provided for the intracellular delivery of a polysaccharide in a therapeutically effective amount. For intracellular delivery, the polysaccharide can be complexed to a cationic polymer, such as a PAE; the polysaccharide can also be delivered in a liposome, microsphere, nanoparticle, etc. As another example the polysaccharide is delivered in free form and is taken into the cell by natural processes and/or is associated with a molecule that normally associates with the polysaccharide in vivo and internalization results. In one embodiment the polysaccharide is provided in an intracellular therapeutically effective amount. The therapeutically effective amount of the polysaccharide can be administered to any subject in need thereof. For example, the therapeutically effective amount can be administered to a subject with or at risk of having a disease characterized by abnormal cell proliferation. Therefore, the method in one aspect is a method for treating a disease characterized by abnormal cell proliferation in a subject.

Diseases characterized by abnormal cell proliferation include cancer, Paget's disease, dermoid cysts, exuberant granulation, sarcoidosis and other granulomatous diseases, tuberculosis, diseases of abberrant inflammation (e.g., rheumatoid arthritis, lupus and spondyloarthropathies), scar formation and associated pathologies (e.g., keloids, spinal cord injury), skin infectious processes (e.g., warts, HPV infection) or retinal detachment. The cancer can be any cancer, including melanoma, hepatic adenocarcinoma, prostatic adenocarcinoma or osteosarcoma. Other cancers include biliary tract cancer; bladder cancer; breast cancer; brain cancer including glioblastomas and medulloblastomas; Burkitt's lymphoma, cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease; lung cancer including small cell lung cancer and non-small cell lung cancer; lymphomas including Hodgkin's disease and lymphocytic lymphomas; neuroblastomas; oral cancer including squamous cell carcinoma; esophageal cancer; ovarian cancer including those arising from epithelial cells, stromal cells, germ cells and mesenchymal cells; pancreatic cancer; prostate cancer, rectal cancer; sarcomas including leiomyosarcoma, rhabdomyosarcoma, liposarcoma, fibrosarcoma, and synovial sarcoma; skin cancer including Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. In one embodiment the cancer is not lymphoma or leukemia. In another embodiment the cancer is not Burkitt's lymphoma.

The invention, therefore, is useful for treating tumor cell proliferation or metastasis in a subject. The terms “treat” and “treating” as used herein refer to inhibiting completely or partially the proliferation or metastasis of a cancer or tumor cell, as well as inhibiting any increase in the proliferation or metastasis of a cancer or tumor cell. Treat or treating also refers to retarding the the proliferation or metastasis of tumor cells in a subject. Additionally, treat or treating may include the elimination or reduction of the symptoms associated with the tumor cell proliferation or metastasis.

A “subject having a cancer” is a subject that has detectable cancerous cells. The cancer may be a malignant or non-malignant cancer. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with the compositions provided the subject may be able to kill the cancer cells as they develop.

The compositions may also be used, for instance, in a method for inhibiting angiogenesis. In this method an effective amount for inhibiting angiogenesis of the composition is administered to a subject in need of treatment thereof. Angiogenesis as used herein is the inappropriate formation of new blood vessels. “Angiogenesis” often occurs in tumors when endothelial cells secrete a group of growth factors that are mitogenic for endothelium causing the elongation and proliferation of endothelial cells which results in a generation of new blood vessels. Several of the angiogenic mitogens are heparin or heparan sulfate binding peptides which are related to endothelial cell growth factors.

The compositions are also useful for inhibiting neovascularization associated with disease such as eye disease. Neovascularization, or angiogenesis, is the growth and development of new arteries. It is critical to the normal development of the vascular system, including injury-repair. There are, however, conditions characterized by abnormal neovascularization, including diabetic retinopathy, neovascular glaucoma, rheumatoid arthritis, and certain cancers. For example, diabetic retinopathy is a leading cause of blindness. There are two types of diabetic retinopathy, simple and proliferative. Proliferative retinopathy is characterized by neovascularization and scarring. About one-half of those patients with proliferative retinopathy progress to blindness within about five years.

Another example of abnormal neovascularization is that associated with solid tumors. It is now established that unrestricted growth of tumors is dependant upon angiogenesis, and that induction of angiogenesis by liberation of angiogenic factors can be an important step in carcinogenesis. For example, basic fibroblast growth factor (bFGF or FGF2) is liberated by several cancer cells and plays a crucial role in cancer angiogenesis. As used herein, an angiogenic condition means a disease or undesirable medical condition having a pathology including neovascularization. Such diseases or conditions include diabetic retinopathy, neovascular glaucoma and rheumatoid arthritis (non-cancer angiogenic conditions). Cancer angiogenic conditions are solid tumors and cancers or tumors otherwise associated with neovascularization such as hemangioendotheliomas, hemangiomas and Kaposi's sarcoma.

Proliferation of endothelial and vascular smooth muscle cells is the main feature of neovascularization. Thus the compositions of the invention are useful for preventing proliferation and, therefore, inhibiting or arresting altogether the progression of the angiogenic condition which depends in whole or in part upon such neovascularization.

Effective amounts of the compositions of the invention are administered to subjects in need of such treatment. Effective amounts are those amounts which will result in a desired reduction in cellular proliferation or metastasis or other therapeutic endpoint without causing other medically unacceptable side effects. The effective amount can refer to the amount of the polysaccharide needed to result in the desired treatment endpoint. The effective amount can also be the amount of the polysaccharide in combination with the cationic polymer, an additional therapeutic agent or some combination thereof that results in the desired treatment endpoint. Such amounts can be determined with no more than routine experimentation. It is believed that doses ranging from 1 nanogram/kilogram to 100 milligrams/kilogram, depending upon the mode of administration, will be effective. The absolute amount will depend upon a variety of factors (including whether the administration is in conjunction with other methods of treatment, the number of doses and individual patient parameters including age, physical condition, size and weight) and can be determined with routine experimentation. It is preferred generally that a maximum dose be used, that is, the highest safe dose according to sound medical judgment. The mode of administration may be any medically acceptable mode including oral, subcutaneous, intravenous, intratumoral, local, etc.

In some aspects of the invention the effective amount of the compositions is that amount effective to prevent invasion of a tumor cell across a barrier. The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

The barrier for the tumor cells may be an artificial barrier in vitro or a natural barrier in vivo. In vitro barriers include but are not limited to extracellular matrix coated membranes, such as Matrigel. Thus the compositions can be tested for their ability to inhibit tumor cell invasion in a Matrigel invasion assay system as described in detail by Parish, C. R., et al., “A Basement-Membrane Permeability Assay which Correlates with the Metastatic Potential of Tumour Cells,” Int. J. Cancer (1992) 52:378-383. Matrigel is a reconstituted basement membrane containing type IV collagen, laminin, heparan sulfate proteoglycans such as perlecan, which bind to and localize bFGF, vitronectin as well as transforming growth factor-β (TGF-β), urokinase-type plasminogen activator (uPA), tissue plasminogen activator (tPA), and the serpin known as plasminogen activator inhibitor type 1 (PAI-1). Other in vitro and in vivo assays for metastasis have been described in the prior art, see, e.g., U.S. Pat. No. 5,935,850, issued on Aug. 10, 1999, which is incorporated by reference. An in vivo barrier refers to a cellular barrier present in the body of a subject.

In some aspects of the invention, polysaccharides that are degraded HSGAGs can be used in the compositions and methods provided herein. These degraded HSGAGs can be obtained after their exposure to a GAG-degrading enzyme, such as heparinase I, heparinase II or heparinase III. Such degraded HSGAGs and GAG-degrading enzymes have been shown to inhibit Burkitt's lymphoma cell growth, see Example below. These degraded HSGAGs can be conjugated to a cationic polymer in some embodiments. In other aspects of the invention the GAG-degrading enzyme can be administered prior to, concurrently with or subsequent to one or more of the compositions provided herein to alter HSGAGs present on the cell surface to elicit the anti-proliferative effects.

It is further provided herein that polysaccharide uptake induced apoptosis is preferential to specific cell types based on internalization rates. Cancer cells, which have a faster endocytic rate than non-cancerous cells, and correspondingly take up polymer-polysaccharide conjugate faster, are typically more susceptible to the effects of the conjugates. While targeting cancer based on endocytic rate alone would likely affect macrophages and neutrophils as well, local delivery could allow for induction of cancer cell death with minimal effects to surrounding tissues. Intratumoral administration can also be used.

Certain cells, such as cancer cells, can also be targeted with the use of a targeting molecule. The compositions provided herein, therefore, can further contain a targeting molecule. The targeting molecule can be physically linked to a polysaccharide or a cationic polymer by any of the methods known in the art. A targeting molecule is any molecule or compound which is specific for a particular cell or tissue and which can be used to direct a polysaccharide; liposome, microsphere or nanparticle containing the polysaccharide; or a conjugate of the polysaccharide with a cationic polymer to the cell or tissue. The targeting molecule can be directed to any of a number of cells to which the administration of the polysaccharide would be beneficial. The targeted cells therefore include non-immunological cells or non-macrophage cells. The targeted cell may also be non-smooth muscle cells. Targeted cells can also be hyperplastic cells. In some embodiments the targeted cells are cells that internalize the polysaccharide or polysaccharide-cationic polymer conjugate within less than 48 hours. In other embodiments the cells internalize the polysaccharide or polysaccharide-cationic polymer conjugate within less than 24 hours. In another embodiment the cells internalize the polysaccharide or polysaccharide-cationic polymer conjugate within less than 12, 10, 8, 6, 4, 2 or fewer hours. Preferably the cells that are targeted have high endocytic rates, such as cancer cells like epithelial cancer cells. The targeting molecule, therefore, can be a molecule which specifically interacts with a cancer cell or a tumor. For instance, the targeting molecule may be a protein or other type of molecule that recognizes and specifically interacts with a tumor antigen. Targeting molecules, therefore, include antibodies or fragments thereof.

Tumor-antigens include Melan-A/MART-1, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, brain glycogen phosphorylase, SSX-1, SSX-2 (HOM-MEL-40), SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, lmp-1, EBV-encoded nuclear antigen (EBNA)-1, and c-erbB-2.

Examples of tumor antigens which bind to either or both MHC class I and MHC class II molecules, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J. Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol. 162:6849-6854, 1999; Boël et al., Immunity 2:167-175, 1995; Van den Eynde et al., J. Exp. Med. 182:689-698, 1995; De Backer et al., Cancer Res. 59:3157-3165, 1999; Jäger et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wölfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140, 1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J Natl Cancer Inst 89:293-300, 1997; Coulie et al., Proc. Natl. Acad. Sci. USA 92:7976-7980, 1995; Wölfel et al., Science 269:1281-1284, 1995; Robbins et al., J. Exp. Med. 183:1185-1192, 1996; Brändle et al., J. Exp. Med. 183:2501-2508, 1996; ten Bosch et al., Blood 88:3522-3527, 1996; Mandruzzato et al., J. Exp. Med. 186:785-793, 1997; Guéuen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Röpke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679, 1999. These antigens as well as others are disclosed in PCT Application PCT/US98/18601.

The compositions provided herein can further comprise an additional therapeutic agent. Additional therapeutic agents include anticancer agents. Anti-cancer agents include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Additional agents that can be included in the compositions provided herein also include GAG-degrading enzymes, uncomplexed HSGAGs, such as heparin, HSGAG fragments produced with GAG-degrading enzymes or FGF. The compositions provided herein may also further include agents that treat the side-effects of radiation therapy, such as anti-emetics, radiation protectants, etc.

The invention also encompasses screening assays for identifying polysaccharides or compositions containing a polysaccharide that can inhibit cell proliferation, promote apoptosis and/or prevent tumor growth. The assays are accomplished by contacting a tumor or isolated tumor cells with the compositions described herein and identifying the compositions that inhibit cell proliferation, promote apoptosis and/or prevent tumor growth.

Kits comprising the compositions discussed herein are also provided. The kits can further include diagnostic agents, such as labels or an additional therapeutic agent.

In general, when administered for therapeutic purposes, the compositions of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients.

The term “physiologically-acceptable” refers to a non-toxic material that is compatible with the biological systems such of a tissue or organism. The physiologically acceptable carrier must be sterile for in vivo administration. The characteristics of the carrier will depend on the route of administration.

In some embodiments the compositions provided are stored in a vial or ampoule.

In other embodiments the compositions provided are sterile.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, which comprise the polysaccharides or complexes provided herein together with one or more pharmaceutically acceptable carriers and optionally other therapeutic ingredients. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with the complexes of the present invention, and with each other, in a manner such that there is no interaction which would substantially impair the desired pharmaceutical efficiency.

A variety of administration routes are available. The particular mode selected will depend, of course, upon the particular polysaccharide or complex selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. A preferred mode of administration is a parenteral route. The term “parenteral” includes subcutaneous injections, intravenous, intramuscular, intraperitoneal, intra sternal injection or infusion techniques. Other modes of administration include oral, mucosal, rectal, vaginal, sublingual, intranasal, intratracheal, inhalation, ocular, transdermal, intratumoral etc. In some embodiments the polysaccharide or complex is delivered locally, such as by local injection.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compositions for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compositions may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may conveniently be presented in unit dosage form and may be prepared by any of the methods well known in the art of pharmacy. All methods include the step of bringing the composition into association with a carrier which constitutes one or more accessory ingredients. In general, the compositions are prepared by uniformly and intimately bringing the compositions into association with a liquid carrier, a finely divided solid carrier, or both, and then, if necessary, shaping the product. The compositions may be stored lyophilized.

Other delivery systems can include time-release, delayed release or sustained release delivery systems. Such systems can avoid repeated administrations of the compositions of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. Nos. 4,748,034 and 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Additional pharmaceutical methods may be employed to control the duration of action. Controlled release preparations may be achieved through the use of polymers to complex or absorb the therapeutic agents of the invention. The controlled delivery may be exercised by selecting appropriate macromolecules (such as polyesters, polyamino acids, polyvinyl, pyrrolidone, ethylenevinylacetate, methylcellulose, carboxymethylcellulose, or protamine sulfate) and methods of incorporation in order to control release. Another possible method to control the duration of action by controlled release preparations is to incorporate the agents provided into particles of a polymeric material such as polyesters, polyamino acids, hydrogels, poly(lactic acid) or ethylene vinyl acetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacrylate) microcapsules, respectively, or in colloidal drug delivery systems, for example, liposomes, albumin microspheres, microemulsions, nanoparticles, and nanocapsules or in macroemulsions.

The compositions provided can also be administered in the form of liposomes. As is known to those skilled in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can also contain stabilizers, preservatives, excipients, and the like. Preferred lipids are phospholipids and phosphatidyl cholines (lecithins), both natural and synthetic. Methods to form liposomes are known in the art. See, e.g., Prescott, ed., METHODS IN CELL BIOLOGY, Volume XIV, Academic Press, New York, N.Y. (1976), p. 33 et seq.

In some embodiments the polysaccharides are delivered in liposomes, microspheres, nanoparticles, etc. for intracellular delivery.

A subject is any human or non-human vertebrate, e.g., dog, cat, horse, cow, pig.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Cellular Uptake of Heparin and Cancer Cell Death

Materials and Methods

Proteins and Reagents

Porcine intestinal mucosa heparin was from Celsus Laboratories (Columbus, Ohio). Fetal Bovine Serum (FBS) was from Hyclone (Logan, Utah). Minimal essential medium (MEM), Dulbecco's modified Eagle medium (DMEM), RPMI-1640, Leibovitz's L-15 medium, phosphate buffered saline (PBS), L-glutamine, and penicillin/streptomycin were obtained from GibcoBRL (Gaithersberg, Md.). Mouse recombinant interleukin-3 (IL-3) was from R & D Systems (Minneapolis, Minn.). B16-BL6, B16-F10, Panc-1, SK-ES-1, and SW-1088 cells were from American Type Culture Collection (Manassas, Va.). Dithiothreitol (DTT) and protease inhibitor cocktail were from Sigma (St. Louis, Mo.). BaF3 cells transfected with fibroblast growth factor (FGF) R1c [16] were generously provided by Dr. David Ornitz (Washington University, St. Louis, Mo.). NIH 3T3 cells were generously provided by Dr. Matthew Nugent (Boston University School of Medicine, Boston, Mass.).

Polymer-Heparin Conjugate Synthesis

Polymers were prepared and conjugated to heparin similar to as described for DNA [11]. Each polymer is named by its composite diacrylate (A-F) and amine (1-20). Briefly, polymers were dissolved with vortexing in 25 mM sodium acetate, pH 5.0, and mixed with heparin in 25 mM sodium acetate, pH 5.0, to produce the desired polymer:heparin ratio (w/w). The mixture was shaken for 30 minutes at room temperature. Complexes were stored at 4° C. until use, which was no greater than 3 hours after conjugation.

Azure A Heparin Binding Assay

The individual effects of heparin and polymer on the Azure A colorimetric assay were first established. Azure A was dissolved in sodium acetate pH 5.0 to produce a 0.2% (w/v) solution. Heparin and each of the 70 polymers from the library soluble in sodium acetate pH 5.0 [11] were dissolved in it to produce solutions ranging between 10 ng/ml and 1 mg/ml. Each sample at each concentration was mixed 1:1 with Azure A in a final volume of 1 ml, mixed thoroughly, and the absorbance was determined at 596 nm [37].

For polymer-Azure A competition assays, 250 μl of 20 μg/ml heparin in 25 mM sodium acetate pH 5.0 was mixed with 250 μl of each of the 70 polymers in 25 mM sodium acetate to yield a final polymer:heparin ratio (w/w) of 1:1, 5:1, 10:1, or 20:1. Each 500 μl solution was shaken for 30 minutes at room temperature to allow for conjugation and supplemented with 500 μl Azure A solution. The resultant solution was incubated for 5 minutes at room temperature, mixed thoroughly, and the absorbance was measured at 596 nm. The free heparin, capable of binding Azure A after polymer:heparin complexes were produced, was determined by comparing the resulting A₅₉₆ to a standard heparin curve.

Cell Culture

Smooth muscle cells (SMCs) were isolated as described [38]. SMCs, bovine aortic endothelial cells (BAECs), NIH 3T3 mouse fibroblast cells, and Panc-1 human pancreatic adenocarcinoma cells were maintained in DMEM supplemented with 10% FBS. B16-BL6 and B16-F10 mouse melanoma cells were maintained in αMEM supplemented with 10% FBS. SK-ES-1 human anablastic osteosarcoma cells were maintained in 5a media supplemented with 15% FBS. SW-1088 human astrocytoma cells were maintained in L-15 media supplemented with 10% FBS. All media was supplemented with 100 μg/ml penicillin, 100 U/ml streptomycin, and 500 μg/ml L-glutamine. Adhesion cells were grown in 75 cm² flasks or 150 cm² dishes at 37° C. in a 5% CO₂ humidified incubator and passaged 2-3 at confluence.

FGFR1c-transfected BaF3 cells were maintained as suspension cultures in RPMI-1640 supplemented with 10% FBS and 500 ng mouse recombinant IL-3. Cultures were grown in 75 cm² flasks at 37° C. in a 5% CO₂ humidified incubator and passaged 1:10 by dilution three times a week.

Conjugate Internalization

Fluorescein-conjugated heparin (Molecular Probes, Eugene, Oreg.) was complexed with polymers as for unconjugated heparin. BAEC, SMC and NIH 3T3 cells were grown until confluent, washed with PBS, treated with 4 ml trypsin-EDTA per 150 cm² tissue culture dish at 37° C. for 3-5 minutes, and collected with 10 ml media. The suspension was pelleted and resuspended in 10 ml proliferation media. Cell concentration was determined with an electronic cell counter and the solution was diluted to 5×10⁴ cells/ml. Wells of 96-well plates were supplemented with 100 μl of cell suspension. For each cell type, three wells per polymer were treated with polymer-heparin conjugates at a 20:1 (w/w) ratio to yield a final heparin concentration of 500 ng/ml. Three wells were treated with an equivalent amount of polymer alone. Three wells for each cell type were treated with fluorescein-labeled heparin. Three wells per cell type contained untreated cells. The plates were incubated for 24 hours at 37° C., 5% CO₂, and visualized with fluorescence microscopy. Conjugates were defined as having enabled heparin internalization if 80% of cells showed fluorescence co-localized with cells in 7 of 10 high powered fields in each of the three wells for the given conjugate, and less than 20% of cells treated with labeled heparin alone in 7 of 10 high powered fields for each of the three wells showed similar co-localization of fluorescence with cells.

To evaluate internalization rates, SMCs, B16-BL6 cells, and B16-F10 cells, were seeded at 5×10⁴ cells/ml in 24-well plates. Three wells for each cell type were treated with 10 μl PBS, A5-fluorescein-labeled heparin conjugates (20:1, w/w; 1 μg/ml), fluorescein-labeled heparin (1 μg/ml), or uncomplexed A5 alone (20 μg/ml). Cells were visualized using fluorescence microscopy every hour for 6 hours, and again after 24 hours. Requirements to define internalization were as described. Digital images were processed using Adobe Illustrator 10.0 and Adobe Photoshop 7.0.

Whole Cell Proliferation Assay

Adhesion cells (B16-F10, B16-BL6, SMCs, BAECs, NIH 3T3, SK-ES-1, Panc-1, and SW-1088) were seeded in 24-well plates at 1 ml/well as well as in 6-well plates at 3 ml/well, both at a density of 5×10⁴ cells/ml. The plates were incubated for 24 hours at 37° C., 5% CO₂. The cells were then washed with PBS and supplemented with media as appropriate. Cells were treated with PBS, heparin, polymer, or polymer-heparin conjugate, in 10 μl quantities at appropriate concentrations. Cells were incubated at 37° C., 5% CO₂ for 72 hours, after which, each well was treated with 500 μl (24 well plates) or 1 ml (6 well plates) trypsin-EDTA for 5-15 minutes at room temperature, after which 400 μl was used to count the cell number with an electronic cell counter. Assays were performed in the presence of 0.1% FBS, supplemented with either PBS, 5 ng/ml FGF2, or 50 mM sodium chlorate. Panc-1 cells were only tested in 10% FBS. Effects of conjugate were normalized to co-treatment without conjugate.

Proliferation assays on transfected BaF3 cells were performed as described [39] with slight modification. Cells were collected from 75 cm² flasks, washed three times with FBS-deficient media, and resuspended into 10 ml FBS-deficient media. Cells were diluted to 1×10⁵ cells/ml based on the reading of an electronic cell counter and plated 1 ml/well in 24-well plates. Wells were treated with PBS, heparin, polymer or polymer:heparin conjugate in 10 μl volumes, and incubated for 72 hours at 37° C., 5% CO₂. Cell counts were determined using an electronic cell counter. Similar conditions were employed as for adherent cells, except FGF2 was applied at a concentration of 50 ng/ml [39]. Effects of conjugate were normalized to the no conjugate condition.

Immunohistochemistry

B16-F10 cultures were washed three times with PBS, dried overnight, and stored at −80° C. until use. Cell cultures were rehydrated in PBS for 10 minutes. After blocking for 20 minutes in PBS. containing 0.1% (w/v) BSA, cultures were incubated with c-Myc-tagged and VSV-tagged anti-HS antibodies (AO4B05, AO4B08, AO4F12, HS4A5, HS4C3, RB4CD12, RB4CB9, RB4EA12, EW4A11, and EW4G2) overnight [40, 41]. Bound antibodies were visualized using either an anti-c-Myc-chicken monoclonal antibody (A21281; Molecular Probes) for 90 minutes, followed by an Alexa 594-conjugated goat anti-chicken IgG antibody for 60 minutes (A11042; Molecular Probes), or a Cy-3-labelled anti-VSV monoclonal antibody (9E10; Sigma). Cultures were washed three times 10 minutes with PBS following each incubation. Finally, cultures were fixed in 100% methanol, dried, and embedded in Mowiol (10% (w/v) in 0.1 M Tris-HCl, pH 8.5/25% (v/v) glycerol/2.5% (w/v) NaN₃). As a control, primary, secondary or conjugated antibodies were omitted. All incubations were performed at ambient temperature (21° C.) with antibody titers of half the dilution factor at which signal was abolished. Photographs were taken, using a constant aperture and shutter time, on a Zeiss Axioskop immunofluorescence microscope (Göttingen, Germany), equipped with a Kodak KAF 1400 CCD. Digital images were processed using Adobe Photoshop 7.0.

Mitogenic Assay

B16-F10 cells were plated in 24 well plates at 5×10⁴ cells/ml in 1 ml/well. Cells were serum starved for 24 hours. Polymer-glycosaminoglycan (GAG) conjugates were added in 10 μl volumes and incubated for 20 hours. Cells were incubated with 1 μCi/ml ³H-thymidine (Perkin Elmer, Wellesley, Mass.) for 4 hours, washed with PBS and treated with 500 μl of 1 M NaOH per well. The contents of each well were transferred to 7 ml scintillation vials containing 5 ml scintillation fluid and counted using a scintillation counter. Data are reported as counts per minute (CPM).

Transcription Factor and Cell Death Assays

To assess the effects on transcription factors, B16-F10 cells were seeded at 5×10⁴ cells/ml in 6 wells plates in propagation media. Cells were serum starved and subsequently treated with PBS, A5 (20 μg/ml), heparin (μg/ml), or A5-heparin formulated at a 20:1 ratio (w/w). ELISA for transcription factors DP-1, E2F-1, E2F-2, p107, Rb, and Sp-1, proceeded as per manufactures' protocol (BD Biosciences, Palo Alto, Calif.). The relative change in transcription factor levels was measured using a spectrophotometric plate reader at 655 nm.

The LDH cytotoxicity assay (Roche, Basel, Switzerland) and the Caspase-3/7 apoptosis assay (Roche) were performed as per manufactures' instructions. B16-F10, B16-BL6, NIH 3T3, Panc-1, SK-ES-1, and SW-1088 cells were grown to confluence in 150 cm² dishes. Cells were trypsinized, pelleted and resuspended in media. Cell concentration was determined using an electronic cell counter. The cell suspension was diluted and cells were plated in 96-well plates as appropriate. The assays proceeded as described and the results were determined using a spectrophotometric plate reader.

Spermine Incorporation Assay

Spermine incorporation was determined as described [19] with slight modification. SMCs, B16-BL6 cells, and B16-F10 cells were seeded at 5×10⁴ cells/ml in 24-well plates in propagation media. Cultures were grown for 24 hours, washed twice with PBS, and supplemented with FBS-deficient media with 5 μM ¹⁴C-spermine (Amersham Biosciences, Piscataway, N.J.). Cells were immediately treated with PBS, heparin (1 μg/ml), A5 (20 μg/ml), or A5:heparin (20:1, w/w). Cells were treated with 5 mM difluoromethylornithine (DFMO), 5 μM spermine, or both DFMO and spermine as controls. After 3, 6, 9, 12, 24, and 48 hour incubations, cells were chilled and washed with ice-cold FBS-deficient media containing 1 mM spermine. Cells were lysed with 0.5 ml NaOH, which was then added to 5 ml scintillation fluid, and incorporation was determined by scintillation counter.

Results

PAEs Bind Heparin

Poly(β-amino ester)s (PAEs) have been previously demonstrated to efficiently bind DNA [10, 11]. The interaction between this class of polymers and deoxyribonucleic acid (DNA) is thought to be primarily mediated through electrostatic interaction between the anionic DNA and the cationic polymers. Azure A is a cationic dye that binds to sulfate groups on heparin [14]. Polymer-heparin binding was examined by determining if polymer could compete with Azure A for binding sites on heparin. The ability of PAEs to displace Azure A was initially examined for five polymers with variable DNA binding efficiencies over a range of polymer:heparin (w/w) ratios. All five polymers displaced heparin. The optimal ratios for these five polymers were at either 5:1 or 20:1. The 70 previously demonstrated water soluble PAEs from an initial screening group of 140 [11] were then tested for their ability to bind heparin. Of the 70 polymers tested, 64 bound heparin to some degree at a 5:1 (w/w) polymer:heparin ratio and all 70 bound heparin at a 20:1 ratio in 25 mM sodium acetate. When dissolved in phosphate buffered saline (PBS), only 57 polymers bound heparin at a 5:1 (w/w) ratio, and 63 at a 20:1 (w/w) ratio. pH affects not only the rate by which PAEs degrade, but also the ability of PAEs to directly bind DNA [10]. The reduced binding of heparin by PAEs at a higher pH is consistent with that found for DNA as well as with the increased degradation rate.

Select PAEs Enable Internalization of Heparin

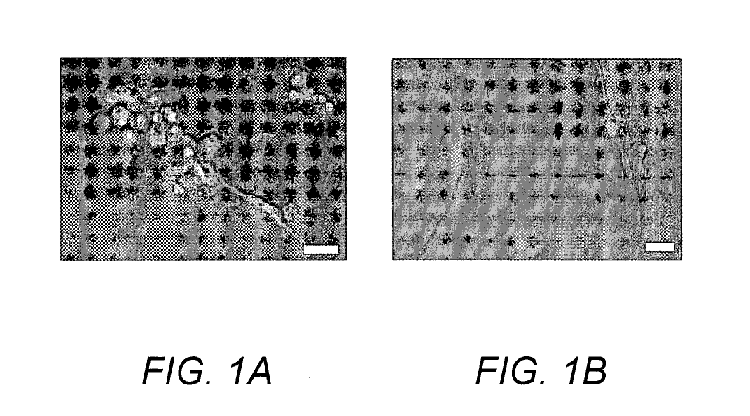

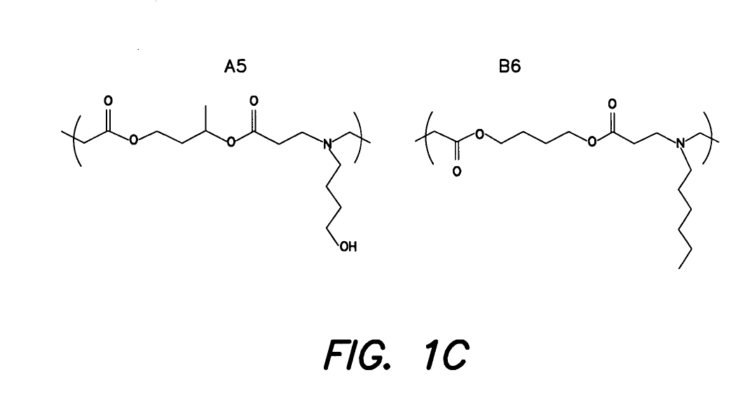

To determine if PAE binding to heparin would enable internalization into cells, as is the case for PAE-DNA conjugates [10, 11], fluorescein-labeled heparin was employed. Conjugates of polymer and fluorescein-labeled heparin were formed in 25 mM sodium acetate for each of the 70 water soluble polymers at a 20:1 (w/w) polymer:heparin ratio. The conjugates were incubated with SMCs, BAECs, and NIH 3T3 cells for 24 hours and internalization was detected by fluorescence microscopy. A group of 14 polymers, that composed of diacrylate “A” and amine “5” (A5), A8, A11, B6, B9, B11, B14, C4, C12, D6, E7, E14, F20, and G5, as exemplified in FIG. 1, enabled passage of heparin across the cell membrane sufficient to meet the criteria detailed in the Materials and Methods. The structures of A5 and B6 can be seen in FIG. 1C. The chemical properties of the various polymers examined and complexes formed with them have been previously reported [11, 15].

Internalized Heparin Inhibits B16-F10 Growth

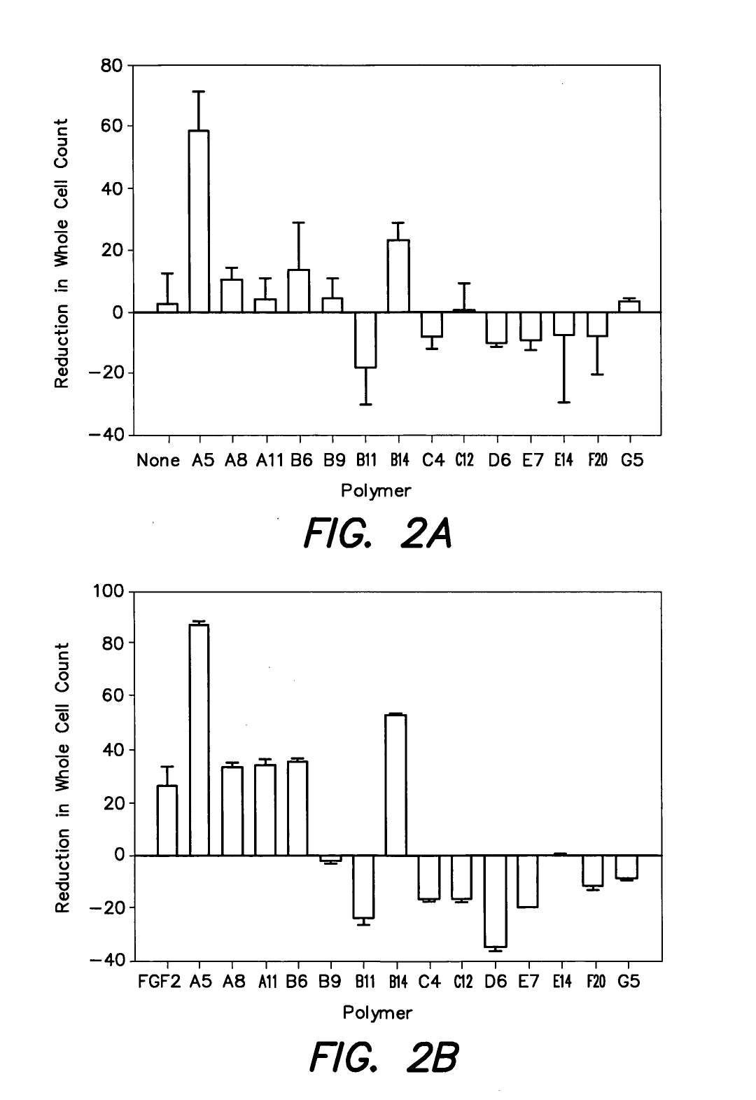

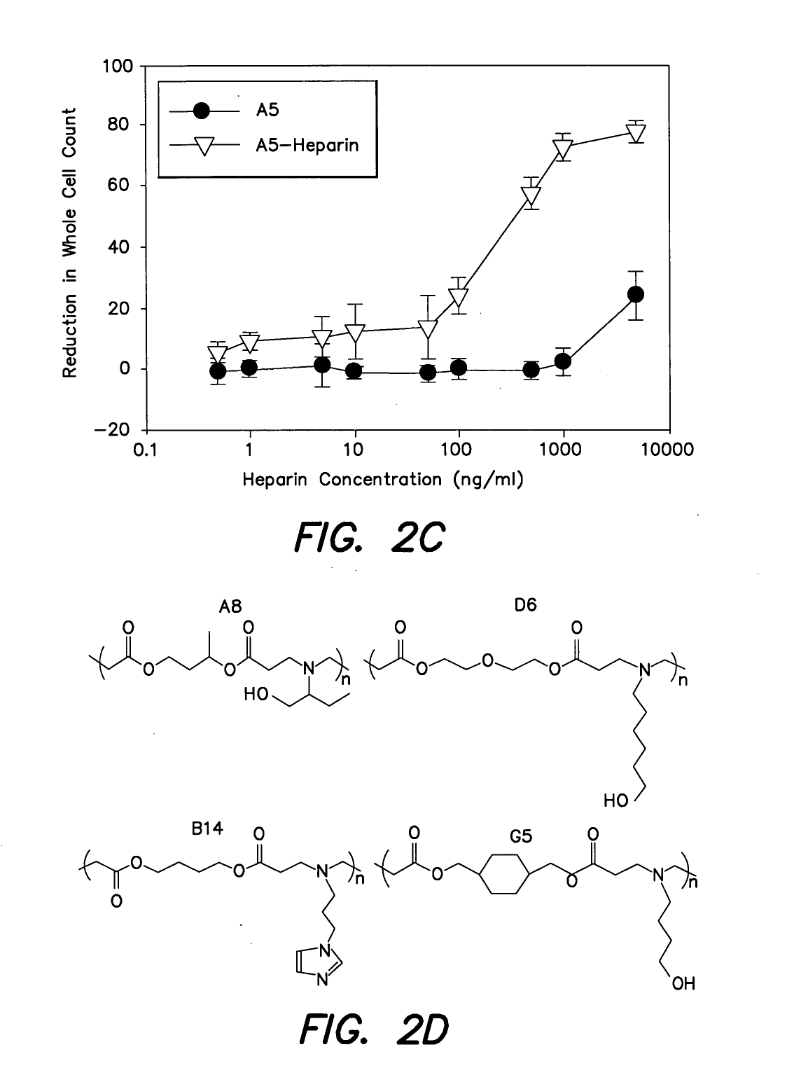

B16-F10 cells were treated with polymer-heparin complexes to investigate if internalized heparin could influence cell processes. Polymer-heparin complexes were formed at a polymer:heparin ratio of 20:1 (w/w) with each of the 14 polymers that enabled heparin internalization. Cells were treated with complexes sufficient to produce a heparin concentration of 500 ng/ml. Internalization of heparin caused a polymer-specific and polymer-dependent response in terms of B16-F10 proliferation (FIG. 2A). A5-heparin induced a 58.28±12.97% reduction in cell number compared to untreated, significantly greater than that induced by any other polymer-heparin conjugate tested (p<0.008). Heparin alone inhibited cell growth 2.40±10.33%.

To examine whether the observed conjugate-induced effects were related to FGF2 cellular mediated responses, each of the 14 polymer-heparin complexes and 10 ng/ml FGF2 were added to cells. In the presence of FGF2, A5-heparin reduced whole cell number by 86.51±1.05% compared to untreated cells. Given that FGF2 alone produced a 26.28±7.23% inhibition, the increased magnitude of the inhibitory effect appears additive (FIG. 2B). FGF2 generally promoted inhibition across polymers in an additive manner. D6 provides a notable exception in that cell number inhibition decreased from −9.51±1.13% to −33.97±1.47%.

The dose dependence of A5-heparin was then determined. The capacity of A5-heparin conjugates to reduce whole cell number increased with concentration (FIG. 2C). 100 μg/ml A5, that added with 5 μg/ml heparin, reduced whole cell number 24.58±7.98% (p<0.004). At 1 μg/ml heparin, A5-hep reduced cell number by 73.14±2.75%. The equivalent amount of polymer was the highest concentration with no significant effect.

Internalized Heparin Affects Cell Processes

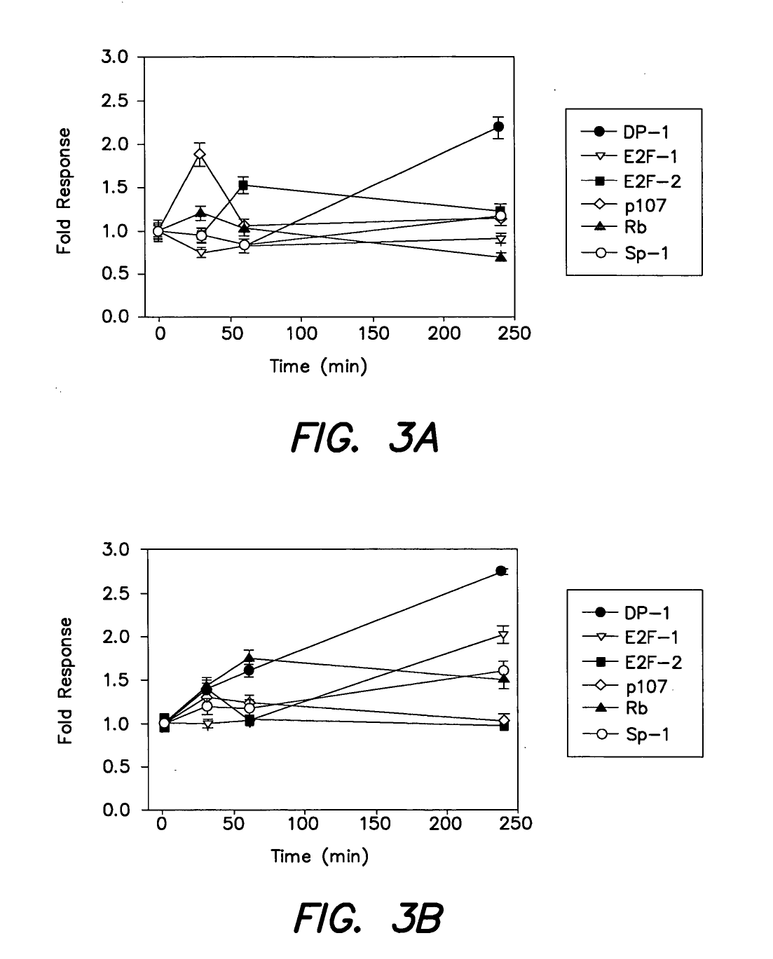

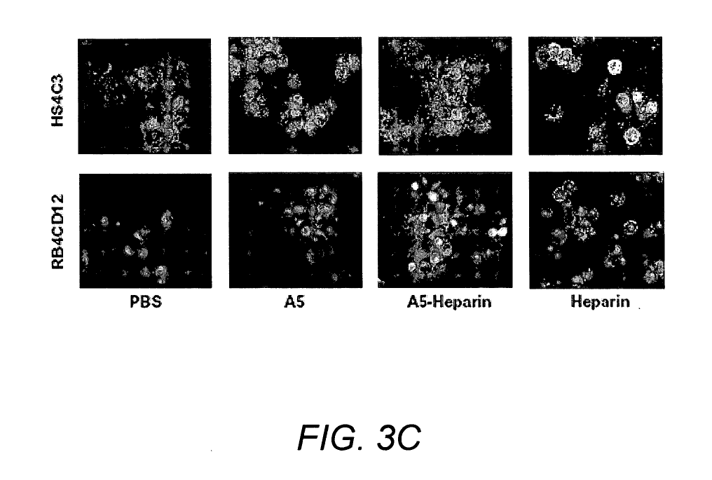

To determine if the conjugate-mediated effects were due to non-specific cytotoxicity, whether specific cell processes were affected was examined. The effects of internalized heparin on six transcription factor levels in B16-F10 cells were determined. A general alteration of specific transcription factors both in the nucleus and the cytoplasm was found (FIGS. 3A and 3B). The most striking effect was seen in DP-1 in the nucleus and the cytoplasm, where levels were elevated 2.18±0.12- and 2.72±0.03-fold respectively. Nuclear E2F-1 and Sp-1 were both initially lower than the control but corrected towards the control. Nuclear p107, Rb, and E2F-2 all showed initial increases compared to control and subsequently declined. After 4 hours, Rb decreased substantially below the level of the control. Cytoplasmic p107 and E2F-2 were initially elevated but returned to near baseline levels. Levels of E2F-1, Rb, and Sp-1 were substantially elevated over time, though Rb did show a relative decrease between 1 hour and 4 hours. The six transcription factors' levels measured showed an average elevation of 1.20- and 1.63-fold in the nucleus and cytoplasm respectively after 4 hours. Without DP-1, the increases are 1.01-fold for nuclear transcription factors and 1.41-fold for cytoplasmic transcription factors.

To examine the occurrence of individual HS epitopes within the heparin/heparan sulfate-like glycosaminoglycans (HSGAGS) present on and around B16-F10 cells, a panel of 10 anti-HS antibodies was used for immunocytological staining of fixed cell cultures. Most antibodies showed a strong staining for HS on the cell surface and in the ECM. Antibodies HS4C3 and RB4CD12 showed differential staining patterns between A5-heparin and heparin or A5 alone (FIG. 3C).

Growth Inhibitory Effects are GAG Specific

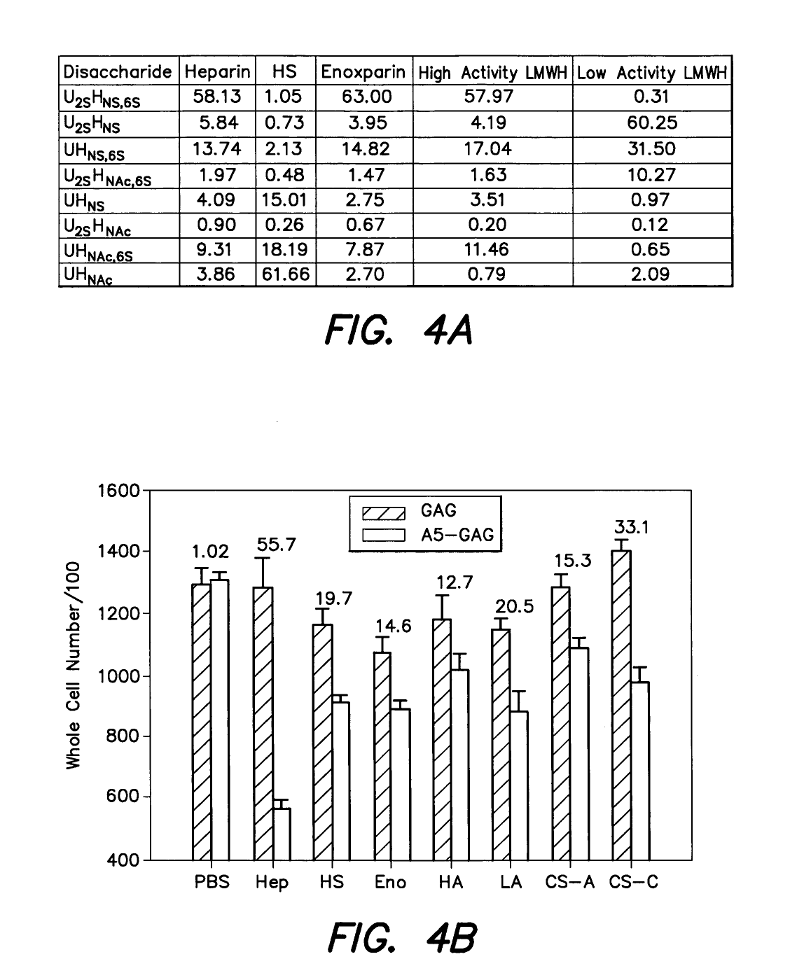

To investigate whether the growth inhibitory effect was specific to heparin or generalized to GAGs of various size, charge, and composition, heparan sulfate (HS), enoxaparin, low molecular weight heparin (LMWH) of two activity levels, and two forms of chondroitin sulfate (CS), were tested for their ability to bind A5 and produce a biological effect in B16-F10 cells through proliferation assays. The composition of the HSGAGs was determined through capillary electrophoresis-based compositional analysis as described [16, 17]. Heparin, enoxaparin, and high activity LMWH had the highest quantities of sulfate groups, averaging 2.32, 2.41, and 2.35 sulfates per disaccharide respectively (FIG. 4A). HS had only 0.43 sulfates per disaccharide. CS-A was primarily 4-O sulfated, with the corresponding peak constituting 98.2% of total peak area. CS-C was primarily 6-O sulfated, but contained some 4-O sulfated disaccharides as well as three forms of disulfated disaccharides. This collection of GAGs therefore allowed for the examination of sulfation degree, length, and saccharide type.

The Azure A binding assay demonstrated that A5 bound to all of each of the GAGs employed at a 20:1 (w/w) A5:heparin ratio in 25 mM sodium acetate. The minimum amount of polymer required for complete binding was higher for GAG species with more sulfates per disaccharide. Correspondingly, A5 (as well as other polymers) bound full length heparin and highly sulfated LMWHs with similar efficiency. Heparin induced the greatest reduction in B 16-F10 cell number (p<5×10⁻⁵; FIG. 4B) of the A5-GAG conjugates (20:1, w/w; 500 ng/ml GAG). The undersulfated HS produced only a 19.70±4.01% reduction compared to that of 53.73±5.80% for heparin. The shorter chain enoxaparin and LMWHs also produced reductions in cell number that were lower in magnitude than full length heparin. Of note, A5 also promoted the maximal cellular mediated effect for LMWHs compared to other polymers that enabled conjugate internalization. Both species of CS each had less of an effect than heparin. The 33.12±5.51% reduction induced by CS-C is significantly greater than the 15.28±4.52% by CS-A (p<0.0002) and that by HS (p<0.001).

Internalized Heparin Promotes a Cell-Specific Response

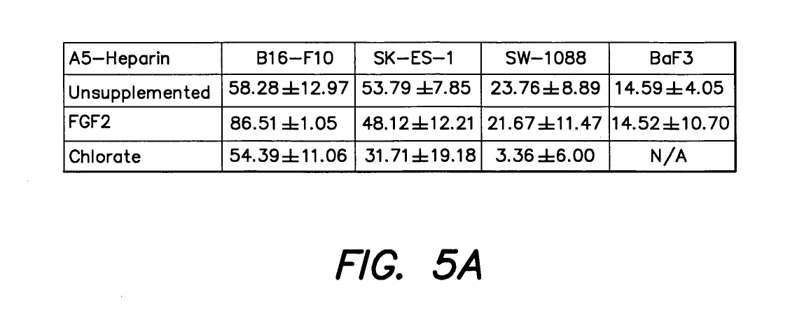

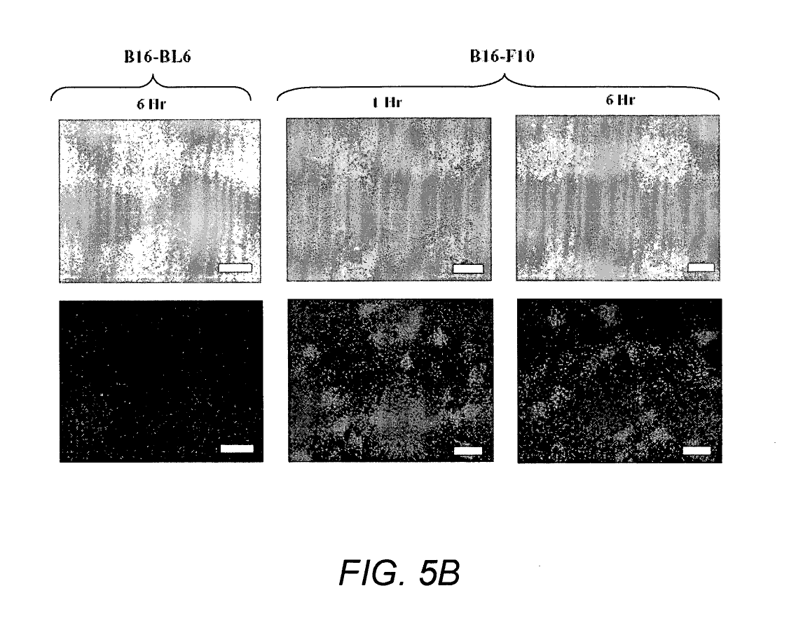

Whether A5-heparin affected other cell types was examined. The proliferative effects of A5-heparin (20:1, w/w; 1 μg/ml heparin) were examined in SMCs, BAECs, FGFR1c-transfected BaF3 cells, SW-1088, SK-ES-1, Panc-1, SK-ES-1, and B16-BL6 by whole cell proliferation. The A5-heparin conjugate had a minimal effect on SMCs (3.84±3.33%), BAECs (−1.09±1.94%), transfected BaF3 cells (14.52±4.05%), B16-BL6 cells (−8.92±12.36%) and Panc-1 cells (−2.74±5.41%), but did elicit a significant reduction in whole cell number in SK-ES-1 (53.79±7.85%) and SW-1088 (23.76±8.89%) cells. Proliferation assays were also performed in the presence of each of 10% FBS, 50 mM sodium chlorate, and 5 ng/ml FGF2 (50 ng/ml for transfected BaF3 cells). The effect of the conjugate was significantly reduced by the presence of FBS. Sodium chlorate, which abrogates cell surface heparin sulfate proteoglycans (HSPGs) [7], reduced the growth inhibitory effects of A5-heparin in SK-ES-1 and SW-1088 cells (FIG. 5A). The effect of A5-heparin in the presence of FGF2 was not significantly different from the summed changes induced separately by conjugate and FGF2.

The cell specific effects of A5-heparin raised the question as to why certain cells were more affected. The results could not be directly attributed to cell turnover rate as transfected BaF3 cells and SMCs, which are not susceptible to A5-heparin conjugate-mediated reductions have a faster turnover rate than SW-1088 cells, which are susceptible. Given that the polymer likely enables internalization by promoting endocytosis [10], whether internalization rates could be the source of the differential effects observed was investigated. Fluorescein-conjugated heparin was used to measured internalization rates in SMCs, B16-BL6 cells and B16-F10 cells. B16-F10 cells showed internalization of heparin within 1 hour (FIG. 5B). Neither SMCs nor B 16-BL6 cells showed significant internalization within 6 hours, though all three cell lines demonstrated internalized conjugate after 24 hours. These results confirm the cell-specific nature of A5-heparin conjugate-mediated inhibition of proliferation and suggest that selectivity is related to the rate of uptake of the complexes.

Internalized Heparin Induces Cell Death

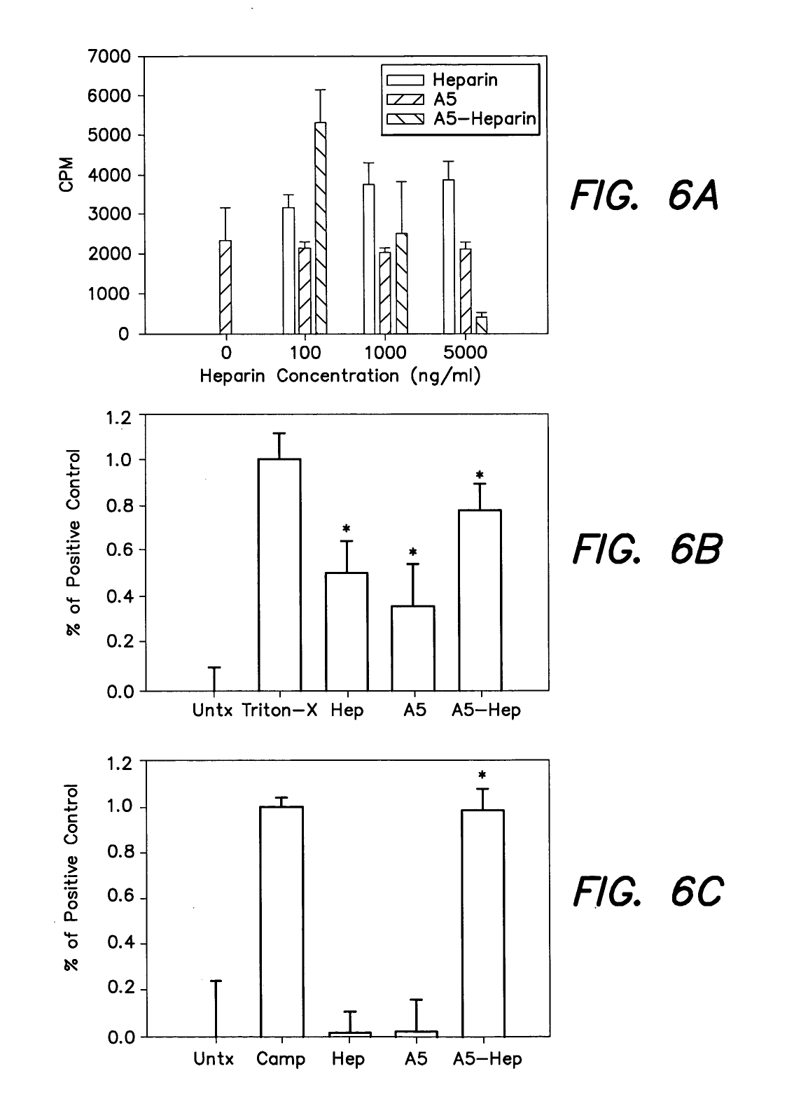

Whether internalization of heparin by A5 affects specific cell processes to reduce whole cell number was determined. ³H-thymidine incorporation was used to measure DNA synthesis in B16-F10 cells after the application of A5-heparin. The mitogenic response followed a dose-response curve wherein low concentrations of A5-heparin promoted ³H-thymidine incorporation while high doses inhibited it (FIG. 6A). None of the equivalent A5 concentrations (20-fold greater than the heparin concentration) including the highest concentration tested, 100 μg/ml, elicited a change in mitogenesis.

The mechanism by which A5-heparin conjugates induced their effects was also examined using a lactic acid dehydrogenase (LDH) cytotoxicity assay and a caspase-3/-7 apoptosis assay. Heparin, A5, and A5-heparin all significantly increased LDH detected compared to the untreated condition (FIG. 6B). Heparin, A5, and A5-heparin elicited responses that were 50.70±13.81%, 35.69±18.94%, and 77.93±11.91% of that caused by Triton-X, the positive control, respectively. A5-heparin conjugate activated caspase-3/-7 levels comparable to camptothecin, the positive control (FIG. 6C). Neither heparin nor A5 alone promoted a significant elevation of caspase activity over PBS, suggesting that the conjugation of A5 and heparin promoted apoptosis in a way not observed with either component alone.

A5-Heparin Promotes Early Spermine Incorporation

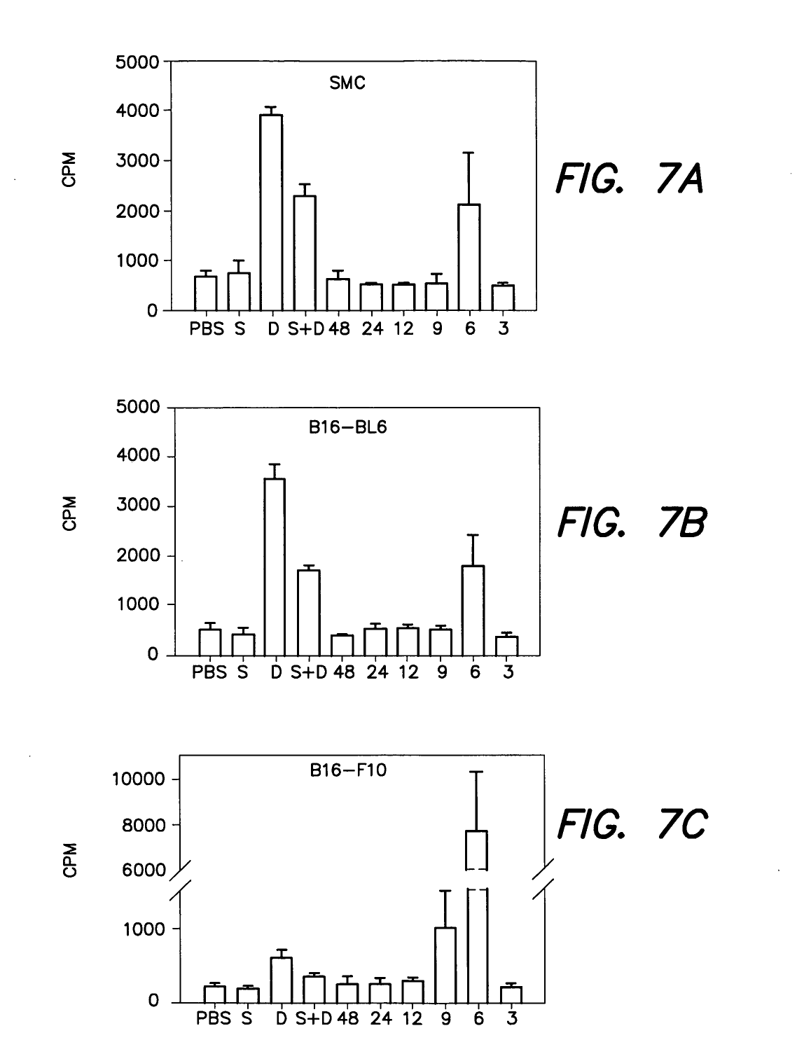

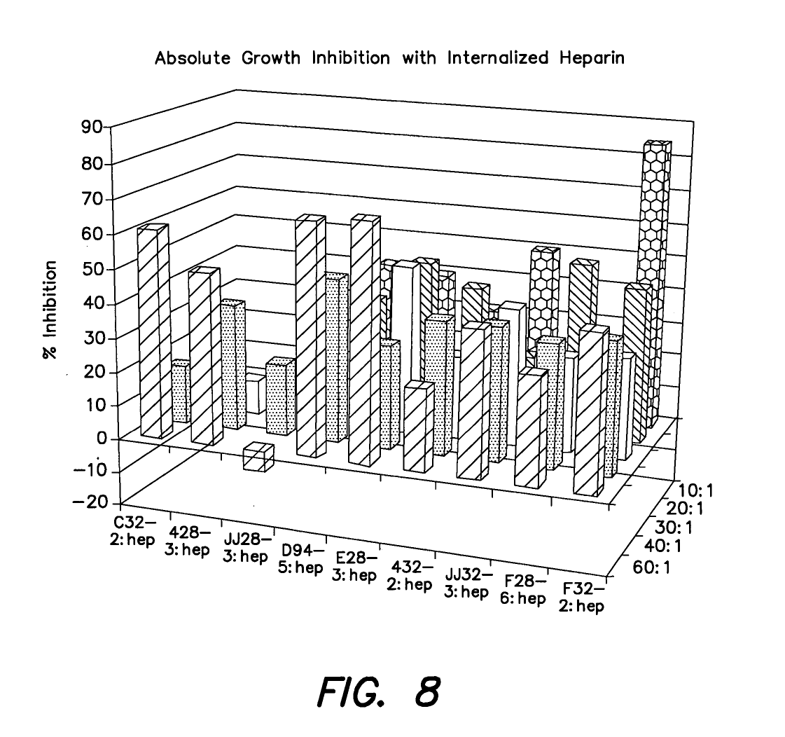

Spermine incorporation was investigated as not only does cell surface HS bind to the spermine transporter which promotes the uptake of spermine, but also cellular proliferation is dependent on an adequate supply of polyamines [18, 19]. To this end, ¹⁴C-spermine incorporation was measured over time subsequent to A5-heparin administration in SMC, B16-BL6, and B 16-F10 cells. SMCs and B16-BL6 cells showed a significant influx of ¹⁴C-spermine at the 6 hour time point (FIG. 7). The magnitude of this effect was 43.97% and 41.83% of that induced by difluoromethylornithine (DFMO) in SMCs and B16-BL6 cells respectively. An influx of ¹⁴C-spermine 19.61-fold greater than with DFMO was observed at 6 hours, however, in B16-F10 cells. Furthermore, 2.00-fold greater incorporation was also evident at the 9 hour time point for B 16-F10 cells.

Heparin Can be Bound and Internalized by Cationic Polymers

The internalization of HSGAGs into cells has been seen as an event involved with specific processes including growth factor signaling and membrane transcytosis. HSGAGs bind to FGF2 and FGFR1 forming a ternary complex that is internalized by endocytosis [7, 8]. HSGAGs also can facilitate membrane transcytosis, such as at the blood-brain barrier [20]. The function of HSGAGs in these cases is to regulate the biological response to and the localization of growth factors. The specific internalization of heparin as a model HSGAG could therefore be used to modulate cell processes involving HSGAGs within the confines of the cell.

Herein, a class of polymers, PAEs, which interact with DNA via a charge-mediated mechanism was utilized. PAEs are an ideal class of polymers for delivery of DNA due to their low toxicity compared to other polymeric methods of DNA delivery, their rapid biodegradability into biologically inert compounds, and their simplicity in synthesis [10, 11]. The primary anionic region of heparin is in the sulfate groups at the N-, 2-O, 3-O, and 6-O positions on the disaccharides that compose heparin. The high quantity of sulfate groups on heparin confers a greater negative charge than DNA [21]. As such, of the 70 water soluble PAEs from a screening library of 140, all bound heparin at a 20:1 w/w ratio in optimal conditions (25 mM sodium acetate, pH 5.0). Substantial binding is similarly facilitated at sub-optimal conditions. A subset of these polymers, however, enable internalization of heparin into cells. The reduced capability of PAEs to enable internalization of heparin compared to that for DNA is not surprising, however, given that a net positive charge, which may trigger endocytosis by promoting interactions with the negatively charged cell membrane, would be more difficult to achieve with a more anionic biopolymer [13]. Correspondingly, the PAEs that mediated the highest levels of DNA internalization had the most positive zeta potentials [15]. The reduced ability of PAEs to enable internalization of heparin compared to DNA is consistent with a net positive charge required for endocytosis. The positive zeta potentials therefore suggest lysosomal escape. Cationic surfaces promote interactions with the lysosome membrane and subsequent release into the cytosol [22]. Apoptotic bodies visible in cultures after addition of fluorescein-heparin conjugated to polymers uniformly exhibited fluorescence (FIG. 1), suggesting even distribution of the conjugates throughout the cytosol. While not being bound by any particular theory, it is thought that the A5-heparin conjugate must escape into the cytosol to significantly alter the activities of transcription factors and caspaces.

Internalized Heparin Affects Cell Processes

The 14 PAEs had distinct levels of response when examined in a whole cell proliferation assay. Polymer A5 was used because the magnitude of change in whole cell number was greatest, suggesting either the highest quantity of heparin internalized or the most robust response induced by the internalized complex. The ability of A5-heparin conjugates to affect whole cell number, transcription factor levels, and the HSGAG epitopes present on and around the cell compared to heparin or A5 alone is consistent with internalization of the complex. Furthermore, complexes formed with PAEs that bind but do not internalize heparin based on assays performed herein, had no effect of whole cell number.

The cellular response to A5-heparin was found to be cell specific (FIG. 5A). In general, non-cancerous cells produced a lower magnitude of effect than cancer cells. The upregulation of huntingtin interacting protein-1, a cofactor in clatharin-mediated endocytosis, has been associated with various epithelial cancers [23, 24]. Endocytic rate has been demonstrated to govern cell sensitivity to exogenous agents [25]. Correspondingly, B16-F10 cells, which exhibited the greatest magnitude of response to A5-heparin conjugates, showed a much faster rate of conjugate internalization than other cells in which less pronounced responses were induced (FIG. 5B). Spermine incorporation, which is greatly increased in susceptible cells, shows maximal effects after 6 hours. SMCs and B16-BL6 cells did not show significant internalization at this time and correspondingly, elicited lower levels of spermine incorporation (FIG. 7). B16-F10s, which internalized A5-heparin conjugates within one hour, showed much greater levels of spermine incorporation. Cell selectivity therefore seems dependent on internalization rate.

Full Length Heparin Promotes the Greatest Biological Response

The biological effect of internalized GAGs is not limited to heparin. Each of heparin, HS, LMWHs, and CS induced some reduction in whole cell number compared to GAG or polymer A5 alone. Full length heparin, however, induced the greatest magnitude of effect. Heparin has the highest charge density of the four full length GAGs tested. High activity LMWH, however, has a similar charge density to, but a smaller biological effect than full length heparin. While the relative amount of each GAG internalized was not quantified, these results suggest that high molecular weights and higher charge densities confer greater activity. Correspondingly, partial digestion of heparin with heparinase I (hepI) [26], which cleaves highly sulfated regions of HSGAGs, prior to conjugation with polymer A5, reduced the magnitude of effect observed. While hepIII digestion, which targets undersulfated regions, also reduced the magnitude of response, the reduction is less than that observed with hepI treatment.

Internalized Heparin Induces Apoptosis

Reduction of whole cell number does not directly explain the mechanism of action or distinguish between either general toxicity or controlled alterations to cell processes. How internalized heparin induced cellular effects was, therefore, analyzed. The mechanism by which internalized heparin induced a cellular mediated response was hypothesized to be by affecting cell processes normally involving heparin, altering cell functions by the degree of negative charge in the cell, or preventing transcription factor binding.

FGF2 has an essential autocrine role in melanoma [27]. Furthermore, the FGF-FGFR complex is stabilized, and downstream signaling is promoted by heparin [28, 29]. The FGF2 system is therefore an ideal approach to examine if internalized heparin alters cell processes normally involving heparin. The effects of A5-heparin conjugates in the presence of FGF2 did not yield a reduction in whole cell number that was distinct from the sum of the independent effects of conjugate and FGF2. The effect of conjugate in the presence of FGF2 was similarly additive in all cell lines examined. Furthermore, the effects of internalized heparin were identical on BaF3 cells as well as those transfected with FGFR1, when normalized to the effects of FGF2 alone. Taken together, these results suggest that the FGF2 pathway is not directly affected by internalized heparin.

The Rb pathway is another critical pathway in the development of melanoma [30]. The mutation of Rb and other tumor suppressor proteins including p107, causes an increase in free E2F family members [31 ]. Internalized heparin led to an upregulation of nuclear E2F-2 and of cytoplasmic E2F-1. Furthermore, Rb while upregulated in the cytoplasm, was downregulated in the nucleus. The levels of p107 were generally unchanged. DP-1 is not typically associated with melanomas, but has been found upregulated in complexes with E2F [32]. Sp-1, which is similarly not thought of as important in melanomas, is upregulated in tumors including glioblastomas [33]. With the exception of elevated levels of Rb found in the cytoplasm, the internalization of heparin promotes a cellular response that is in accordance with promoting melanoma growth.

The internalization of heparin places a substantial quantity of a highly charged compound into cells. While this could adversely affect cells through a non-specific process, controlled internalization of 0.15 M trehalose actually protects cells from environmental changes [34]. With the addition of 1 μg heparin to 5×10⁴ cells, each cell could receive up to 20 pg of internalized heparin, or ˜.13 M heparin, suggesting against a purely osmotic effect. Furthermore, HA-LMWH, which has the same charge density as full length heparin, has a much lower capacity to reduce whole cell number. Therefore, non-specific charge mediated effects do not appear to be the source of the biological response observed.

Oligosaccharides have been previously demonstrated to bind transcription factors [35]. Heparin is additionally used to assess the binding strength of delivery systems to DNA because the greater charge density of heparin can compete charged molecules off of DNA. Transcription factors in both the cytosol and nucleus were found to be upregulated. Since an ELISA technique was used to quantify transcription factor levels, heparin could lead to an apparent increase in transcription factor levels by competing the transcription factors off of DNA and freeing binding sites. Antithrombin III, however, prevents NF-κB activation and the subsequent production of growth factors and cytokines in a heparin dependent manner [36]. Internalized heparin, therefore, likely inhibits transcription factor activity either through preferential binding over DNA or by inhibition of their activation. The alterations in mitogenic response and caspase-3/-7 activity (FIG. 6) were consistent with specific cell processes being affected to induce apoptosis. These results suggest that internalized heparin reduces cell number by inducing apoptotic cell death through a transcription-factor mediated mechanism.

Above are details of a mechanism by which, for example, large, highly charged polysaccharides can be delivered into cells. This delivery induces a cell specific apoptotic response, based primarily on the rate at which complexes are internalized. Since some cancers have a higher endocytic rate, the use of internalized heparin offers an efficient treatment approach. Additionally, as heparin can bind several growth factors and cytokines, delivery of heparin could serve as a platform for the development of combination therapies to treat cancer.

### REFERENCES FOR EXAMPLE 1

1. Sasisekharan, R., Shriver, Z., Venkataraman, G., and Narayanasami, U. (2002). Roles of heparan-sulphate glycosaminoglycans in cancer. Nat Rev Cancer 2, 521-528.
2. Perrimon, N., and Bernfield, M. (2000). Specificities of heparan sulphate proteoglycans in developmental processes. Nature 404, 725-728.
3. Conrad, H. E. (1998). Heparin-Binding Proteins (San Diego: Academic Press).
4. Esko, J. D., and Lindahl, U. (2001). Molecular diversity of heparan sulfate. J Clin Invest 108, 169-173.
5. Blackhall, F. H., Merry, C. L., Davies, E. J., and Jayson, G. C. (2001). Heparan sulfate proteoglycans and cancer. Br J Cancer 85, 1094-1098.
6. Liu, D., Shriver, Z., Venkataraman, G., El Shabrawi, Y., and Sasisekharan, R. (2002). Tumor cell surface heparan sulfate as cryptic promoters or inhibitors of tumor growth and metastasis. Proc Natl Acad Sci USA 99, 568-573.
7. Sperinde, G. V., and Nugent, M. A. (2000). Mechanisms of fibroblast growth factor 2 intracellular processing: a kinetic analysis of the role of heparan sulfate proteoglycans. Biochemistry 39, 3788-3796.
8. Pellegrini, L., Burke, D. F., von Delft, F., Mulloy, B., and Blundell, T. L. (2000). Crystal structure of fibroblast growth factor receptor ectodomain bound to ligand and heparin. Nature 407, 1029-1034.
9. Hsia, E., Richardson, T. P., and Nugent, M. A. (2003). Nuclear localization of basic fibroblast growth factor is mediated by heparan sulfate proteoglycans through protein kinase C signaling. J Cell Biochem 88, 1214-1225.
10. Lynn, D. M., and Langer, R. (2000). Degradable Poly(β-amino esters): Synthesis, Characterization, and Self-assembly with plasmid DNA. J. Am. Chem. Soc 122, 10761-10768.
11. Lynn, D. M., Anderson, D. G., Putnam, D., and Langer, R. (2001). Accelerated Discovery of Synthetic Transfection Vectors: Parallel Synthesis and Screening of a Degradable Polymer Library. J. Am. Chem. Soc. 123, 8155-8156.
12. Brazeau, G. A., Attia, S., Poxon, S., and Hughes, J. A. (1998). In vitro myotoxicity of selected cationic macromolecules used in non-viral gene delivery. Pharm Res 15, 680-684.
13. Kabanov, A. V., and Kabanov, V. A. (1995). DNA complexes with polycations for the delivery of genetic material into cells. Bioconjug Chem 6, 7-20.
14. Wang, L., Malsch, R., and Harenberg, J. (1997). Heparins, low-molecular-weight heparins, and other glycosaminoglycans analyzed by agarose gel electrophoresis and Azure A-silver staining. Semin Thromb Hemost 23, 11-16.
15. Akinc, A., Lynn, D. M., Anderson, D. G., and Langer, R. (2003). Parallel synthesis and biophysical characterization of a degradable polymer library for gene delivery. J Am Chem Soc 125, 5316-5323.
16. Berry, D., Kwan, C. P., Shriver, Z., Venkataraman, G., and Sasisekharan, R. (2001). Distinct heparan sulfate glycosaminoglycans are responsible for mediating Fibroblast Growth Factor-2 biological activity though different Fibroblast Growth Factor Receptors. Faseb J 15, 1422-1424.
17. Berry, D., Shriver, Z., Natke, B., Kwan, C., Venkataraman, G., and Sasisekharan, R. (2003). Heparan sulfate glycosaminoglycan derived from endothelial cells and smooth muscle cells differentially modulate Fibroblast Growth Factor-2 biological activity through Fibroblast Growth Factor Receptor-1. Biochem J 372, 1-9.
18. Belting, M., Havsmark, B., Jonsson, M., Persson, S., and Fransson, L. A. (1996). Heparan sulphate/heparin glycosaminoglycans with strong affinity for the growth-promoter spermine have high antiproliferative activity. Glycobiology 6, 121-129.
19. Belting, M., Borsig, L., Fuster, M. M., Brown, J. R., Persson, L., Fransson, L. A., and Esko, J. D. (2002). Tumor attenuation by combined heparan sulfate and polyamine depletion. Proc Natl Acad Sci USA 99, 371-376.
20. Deguchi, Y., Okutsu, H., Okura, T., Yamada, S., Kimura, R., Yuge, T., Furukawa, A., Morimoto, K., Tachikawa, M., Ohtsuki, S., Hosoya, K., and Terasaki, T. (2002). Internalization of basic fibroblast growth factor at the mouse blood-brain barrier involves a heparan sulfate proteoglycan. J Neurochem 83, 381-389.
21. Piepkorn, M. W., and Daynes, R. A. (1983). Heparin effect on DNA synthesis in a murine fibrosarcoma cell line: influence of anionic density. J Natl Cancer Inst 71, 615-618.
22. Panyam, J., Zhou, W. Z., Prabha, S., Sahoo, S. K., and Labhasetwar, V. (2002). Rapid endo-lysosomal escape of poly(DL-lactide-co-glycolide) nanoparticles: implications for drug and gene delivery. Faseb J 16, 1217-1226.
23. Ross, T. S., and Gilliland, D. G. (1999). Transforming properties of the Huntingtin interacting protein 1/platelet-derived growth factor beta receptor fusion protein. J Biol Chem 274, 22328-22336.
24. Rao, D. S., Hyun, T. S., Kumar, P. D., Mizukami, I. F., Rubin, M. A., Lucas, P. C., Sanda, M. G., and Ross, T. S. (2002). Huntingtin-interacting protein 1 is overexpressed in prostate and colon cancer and is critical for cellular survival. J Clin Invest 110, 351-360.
25. Bazill, G. W., and Dexter, T. M. (1990). Role of endocytosis in the action of ether lipids on WEHI-3B, HL60, and FDCP-mix A4 cells. Cancer Res 50, 7505-7512.
26. Berry, D., Shriver, Z., Natke, B., Kwan, C. P., Venkataraman, G., and Sasisekharan, R. (2003). Heparan sulphate glycosaminoglycans derived from endothelial cells and smooth muscle cells differentially modulate fibroblast growth factor-2 biological activity through fibroblast growth factor receptor-1. Biochem J 373, 241-249.
27. Graeven, U., Rodeck, U., Karpinski, S., Jost, M., Philippou, S., and Schmiegel, W. (2001). Modulation of angiogenesis and tumorigenicity of human melanocytic cells by vascular endothelial growth factor and basic fibroblast growth factor. Cancer Res 61, 7282-7290.
28. Venkataraman, G., Sasisekharan, V., Herr, A. B., Ornitz, D. M., Waksman, G., Cooney, C. L., Langer, R., and Sasisekharan, R. (1996). Preferential self-association of basic fibroblast growth factor is stabilized by heparin during receptor dimerization and activation. Proc Natl Acad Sci USA 93, 845-850.
29. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible fo FGF receptor dimerization, activation, and cell proliferation. Cell 79, 1015-1024.
30. Kannan, K., Sharpless, N. E., Xu, J., O'Hagan, R. C., Bosenberg, M., and Chin, L. (2003). Components of the Rb pathway are critical targets of UV mutagenesis in a murine melanoma model. Proc Natl Acad Sci USA 100, 1221-1225.
31. Halaban, R. (1999). Melanoma cell autonomous growth: the Rb/E2F pathway. Cancer Metastasis Rev 18, 333-343.
32. Choubey, D., and Gutterman, J. U. (1997). Inhibition of E2F-4/DP-1-stimulated transcription by p202. Oncogene 15, 291-301.
33. Konduri, S., Lakka, S. S., Tasiou, A., Yanamandra, N., Gondi, C. S., Dinh, D. H., Olivero, W. C., Gujrati, M., and Rao, J. S. (2001). Elevated levels of cathepsin B in human glioblastoma cell lines. Int J Oncol 19, 519-524.
34. Eroglu, A., Toner, M., and Toth, T. L. (2002). Beneficial effect of microinjected trehalose on the cryosurvival of human oocytes. Fertil Steril 77, 152-158.
35. Dudas, J., Ramadori, G., Knittel, T., Neubauer, K., Raddatz, D., Egedy, K., and Kovalszky, I. (2000). Effect of heparin and liver heparan sulphate on interaction of HepG2-derived transcription factors and their cis-acting elements: altered potential of hepatocellular carcinoma heparan sulphate. Biochem J 350 Pt 1, 245-251.
36. Oelschlager, C., Romisch, J., Staubitz, A., Stauss, H., Leithauser, B., Tillmanns, H., and Holschermann, H. (2002). Antithrombin III inhibits nuclear factor kappaB activation in human monocytes and vascular endothelial cells. Blood 99, 4015-4020.
37. Klein, M. D., Drongowski, R. A., Linhardt, R. J., and Langer, R. S. (1982). A colorimetric assay for chemical heparin in plasma. Anal Biochem 124, 59-64.
38. Nugent, M. A., and Edelman, E. R. (1992). Kinetics of basic fibroblast growth factor binding to its receptor and heparan sulfate proteoglycan: a mechanism for cooperactivity. Biochemistry 31, 8876-8883.
39. Padera, R., Venkataraman, G., Berry, D., Godvarti, R., and Sasisekharan, R. (1999). FGF-2/fibroblast growth factor receptor/heparin-like glycosaminoglycan interactions: a compensation model for FGF-2 signaling. Faseb J 13, 1677-1687.
40. van Kuppevelt, T. H., Dennissen, M. A., van Venrooij, W. J., Hoet, R. M., and Veerkamp, J. H. (1998). Generation and application of type-specific anti-heparan sulfate antibodies using phage display technology. Further evidence for heparan sulfate heterogeneity in the kidney. J Biol Chem 273, 12960-12966.
41. van de Westerlo, E. M., Smetsers, T. F., Dennissen, M. A., Linhardt, R. J., Veerkamp, J. H., van Muijen, G. N., and van Kuppevelt, T. H. (2002). Human single chain antibodies against heparin: selection, characterization, and effect on coagulation. Blood 99, 2427-2433.

### Example 2

### Heparin Inhibits Tumor Growth

Materials and Methods

Proteins and Reagents

FBS was from Hyclone (Logan, Utah). L-glutamine, penicillin/streptomycin, PBS, and Trizol reagent were obtained from GibcoBRL (Gaithersberg, Md.). Porcine intestinal mucosa heparin was from Celsus Laboratories (Columbus, Ohio). Recombinant human FGF2 was a gift from Scios, Inc. (Mountainview, Calif.). Recombinant heparinases were produced as described (1). Rabbit α-FGF2, rabbit α-FGFR1, mouse α-FGFR3, rabbit α-Erk1, rabbit α-Erk2, goat α-phospho-Erk1/2 (Thr 202/Tyr 204), rabbit α-goat conjugated to horse radish peroxidase (HRP), and goat α-rabbit conjugated to HRP were from Santa Cruz Biotechnology (Santa Cruz, Calif.). Kinase inhibitors LY294002, PD98059, SB203580, and U0126 were from Promega (Madison, Wis.).

Cell Culture

PC-3 cells (American Type Culture Collection, Manassas, Va.) were maintained in Ham's F12K medium (American Type Culture Collection) supplemented with 1.5 mg/mL sodium bicarbonate, 100 μg/ml penicillin, 100 U/ml streptomycin, 500 μg/ml L-glutamine and 10% FBS. Cells were grown in 75 cm² flasks at 37° C. in a 5% CO₂ humidified incubator. Confluent cultures were split 1:3 to 1:6, two to three times per week.

Proliferation Assays

PC-3 cells were grown until confluence in 75 cm² flasks. Each flask was washed with 20 ml PBS, and treated with 3 ml trypsin-EDTA at 37° C. for 3-5 minutes, until cells detached. Cells were centrifuged for 3 min at 195×g. The supernatant was aspirated, and the cells were resuspended in 10 ml media. The cell density was measured using an electronic cell counter, and the suspension was diluted to 50,000 cells/ml. The suspension was plated 1 ml/well into 24-well tissue culture plates. After a 24 hour incubation in a 5% CO₂, 37° C. humidified incubator, the media was aspirated, the wells were washed with serum free media, and the cells were supplemented with media containing 0.1% FBS and incubated for 24 hours. Cells were sequentially treated with antibodies, kinase inhibitors, heparin, and FGF2. Antibodies to FGF2, FGFR1, or FGFR3 were added to yield a final dilution of 1:100. Kinase inhibitors were added sufficient to yield final concentrations of 50 μM LY294002, 20 μM PD98059, 1 μM SB203580, and 20 μM U0126. Heparin was added at 500 ng/ml unless otherwise noted. To produce heparin digests, heparin was treated with PBS, hepI, or hepIII for 30 minutes, and boiled for 30 minutes. prior to addition to cells. Digestion was verified by UV spectroscopy at 232 nm. FGF2 was added at 100 ng/ml unless otherwise specified. Cells were then incubated for 72 hours. Wells were then washed twice with PBS and treated with 0.5 ml trypsin-EDTA/well and incubated for 10 minutes at 37° C. Whole cell number was determined using an electronic cell counter. Data were averaged over three experiments, each consisting of four wells per condition.

RT-PCR

5 μg of total RNA was isolated from PC-3 cells using Trizol reagent (Life Tech, Rockville, Md.) followed by reverse transcription with random hexamers. Specific oligomers were designed based on the published sequences of FGFR isoforms in order to detect their expression. Sequences of primer pairs corresponding to distinct FGFR isoforms were as follows: FGFR1b: 5′-TGG AGC AAG TGC CTC CTC-3′ (SEQ ID NO:1) and 5′-ATA TTA CCA CTT CGA TTG GTC-3′ (SEQ ID NO:2); FGFR1c: 5′-TGG AGC TGG AAG TGC CTC CTC-3′ (SEQ ID NO:3) and 5′-GTG ATG GGA GAG TCC GAT AGA-3′ (SEQ ID NO:4); FGFR2b: 5′-GTC AGC TGG GGT CGT TTC ATC-3′ (SEQ ID NO:5) and 5′-CTG GTT GGC CTG CCC TAT ATA-3′ (SEQ ID NO:6); FGFR2c: 5′-GTC AGC TGG GGT CGT TTC ATC-3′ (SEQ ID NO:7) and 5′-GTG AAA GGA TAT CCC AAT AGA-3′ (SEQ ID NO:8); FGFR3b: 5′ GTA GTC CCG GCC TGC GTG CTA-3′ (SEQ ID NO:9) and 5′-GAC CGG TTA CAC AGC CTC GCC-3′ (SEQ ID NO:10); FGFR3c: 5′-GTA GTC CCG GCC TGC GTG CTA-3′ (SEQ ID NO:11) and 5′-TCC TTG CAC AAT GTC ACC TTT-3′ (SEQ ID NO:12); and FGFR4: 5′-CCC TGC CGG GAT CGT GAC CCG-3′ (SEQ ID NO:13) and 5′-TCG AAG CCG CGG CTG CCA AAG-3′ (SEQ ID NO:14). To control for total cell protein, RT-PCR was also performed on β-actin using the primers 5′-GCC AGC TCA CCA TGG ATG ATG ATA T-3′ (SEQ ID NO:15) and 5′-GCT TGC TGA TCC ACA TCT GCT GGA A-3′ (SEQ ID NO:16). PCR was performed using the Advantage-GC cDNA kit from Clontech as per manufacturer's instructions (Palo Alto, Calif.). Prior to experimental use, primers were confirmed to detect and have specificity towards given FGFR isoforms using BaF3 cells transfected with various FGFRs.

Whole Cell ELISA

ELISA was performed on whole cells to determine a quantifiable relative level of kinase activity. PC-3 cells were grown until confluence in 75 cm² flasks. Each flask was washed with 20 ml PBS, and treated with 3 ml trypsin-EDTA at 37° C. for 3-5 minutes, until cells detached. Cells were centrifuged for 3 min at 195×g. The supernatant was aspirated, and the cells were resuspended in 10 ml media. The cell density was measured using an electronic cell counter, and the suspension was diluted to 50,000 cells/ml. 100 mm dishes were supplemented with 10 ml cell suspension per dish. After a 24 hour incubation the media was aspirated, the dishes washed with serum free media, and the cells supplemented with media containing 0.1% FBS and incubated for 24 hours. Dishes were treated with PBS, 10 ng/ml FGF2, or 500 ng/ml heparin for 5, 15, or 60 minutes. Media was then aspirated, the cells were washed with 10 ml PBS, and each dish was treated with 5 ml Trizol reagent. Cell extract was added to 96-well plates previously incubated for 1 hour with primary antibodies to Erk1, Erk2 or phospho-Erk1/2. The cell extract was incubated on the plates for 1 hour, after which, wells were washed twice, and supplemented with the same primary antibody (1:200) as was in the well. Wells were again incubated 1 hour, washed twice, and then treated with secondary antibody (1:500). Goat α-rabbit-HRP was used for Erk1 and Erk2, while rabbit α-goat-HRP was used for phospho-Erk1/2. Plates were incubated for 30 minutes, washed twice, and incubated in with TMB (tetra methyl benzidine) One Solution (Promega). The reaction was quenched with 1 M HCl, and the plates were analyzed using a UV plate reader at 450 nm. Data were quantified by comparing to a standardized curve with varying concentrations of untreated cells.

Measurement of Anti-Coagulant Activity

After completion of the experiments, mice were sacrificed by CO₂ asphyxiation. ˜500 ml blood was collected by cardiac puncture. Blood was centrifuged and plasma extracted. The anti-coagulant effect of treatments was assessed by measuring activities of plasma factors Xa and IIa. Plasma was diluted 1:150 in PBS to a final volume of 90 μl and treated with 600 ng of either chromogenic substrate for factor Xa or for factor IIa (Sigma, St. Louis Mo.) as appropriate in 10 μl PBS. Change in absorbance per second was measured at 405 nm.

Results

Heparin Inhibits FGF2-Mediated Proliferation of PC-3 Cells

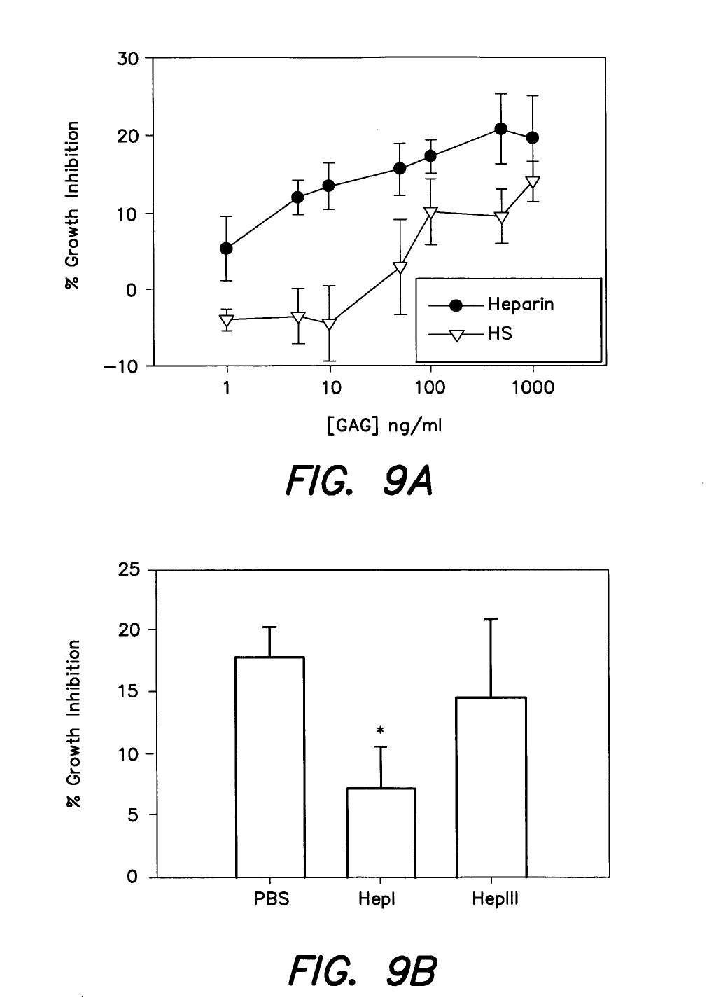

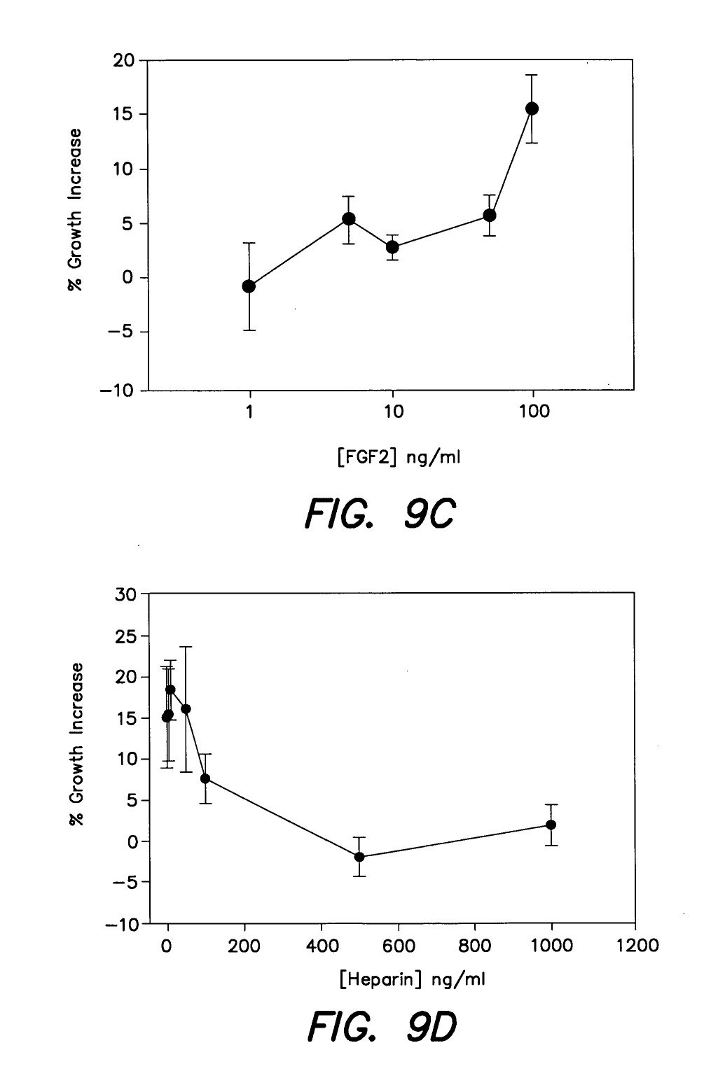

It was determined that heparin altered the proliferative capacity of PC-3 cells. Heparin has been demonstrated to impact PC-3 prostatic adenocarcinoma cell adhesion to endothelial monolayers, an important step in cancer development (2). Furthermore, PC-3 cells elaborate FGF2 (3). Heparin induced a dose-dependent inhibition of PC-3 cell proliferation, reaching a maximum of ˜16% at 100 ng/ml (FIG. 9A). Heparan sulfate, exhibited lower potency, but at substantially higher doses elicited an equivalent inhibitory affect. To confirm this finding, heparin was partially digested with heparinases, and added to PC-3 cells. HepI digestion reduced the inhibitory capacity of heparin by ˜60% (p<0.0002), while hepIII had no significant effect (FIG. 9B).

Since PC-3 cells elaborate FGF2, the effect of FGF2 on proliferation was established. Doses of 50 ng/ml and lower did not promote significant proliferation (FIG. 9C). However, 100 ng/ml FGF2 caused a ˜15% increase in proliferation (p<0.0005). To investigate if heparin could neutralize the FGF2 mediated proliferation, PC-3 cells were treated with 100 ng/ml FGF2 in the presence of increasing concentrations of heparin. FGF2 mediated proliferation was evident at heparin concentrations of 100 ng/ml and less, though the magnitude of proliferation was reduced at 100 ng/ml (FIG. 9D). At 500 ng/ml and 1000 ng/ml heparin, the capacity for FGF2 to induce a proliferative response was eliminated.

FGF2 Induces PC-3 Proliferation through FGFR1c

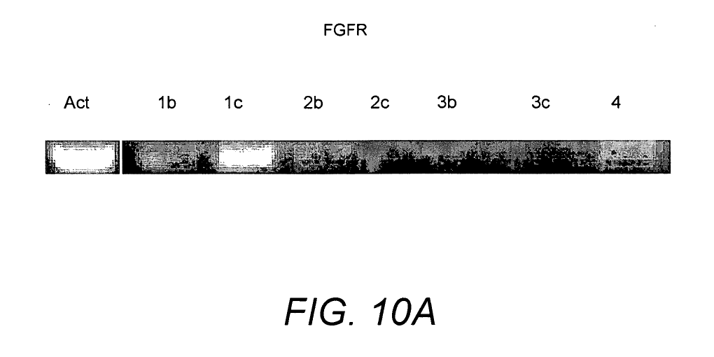

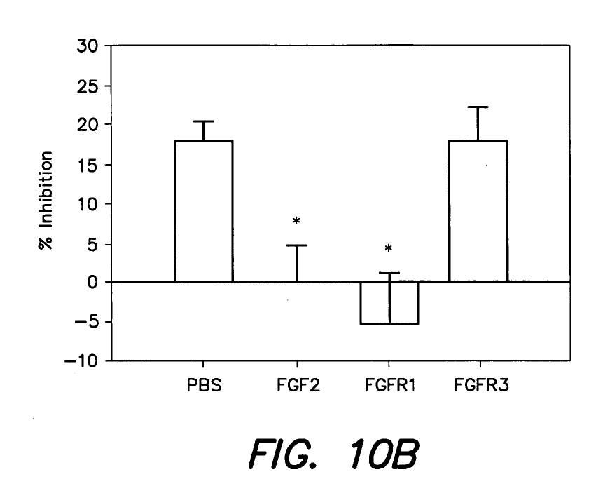

To examine how FGF2 induces a proliferative response, RT-PCR was performed to profile the FGFR isoforms expressed. PC-3 cells were found to contain message for FGFR1c (FIG. 10). Additionally, FGFR1b, FGFR2b, and FGFR4 may be expressed. To examine whether FGF2 signaling through FGFRs could be affected with heparin treatment, PC-3 cells were treated with heparin and antibodies to FGF2, FGFR1 and FGFR3, and normalized to antibody in the absence of heparin. Antibodies to FGF2 and FGFR1 abrogated the capacity of heparin to induce an inhibitory response. FGFR3 did not alter the capacity of heparin to inhibit cell growth.

Heparin Inhibits FGF2 Signaling

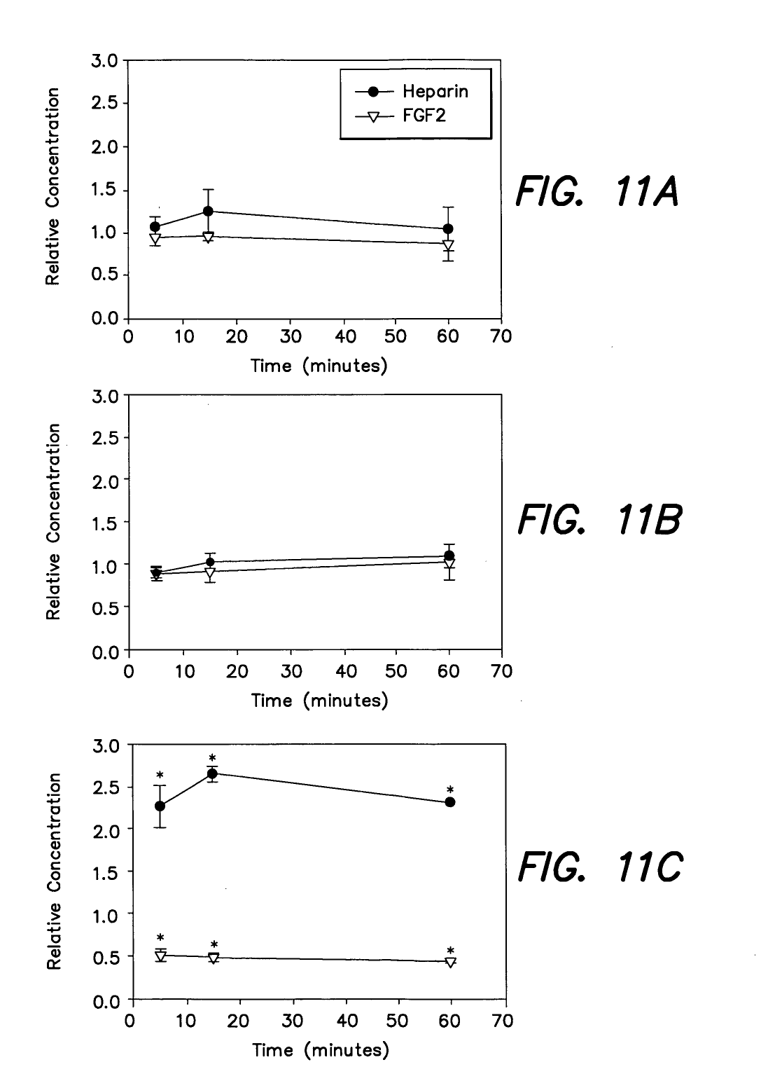

Given that PC-3 cells express a receptor through which FGF2 induces a cell mediated response, how FGF2 and heparin affect FGF2 downstream signaling was characterized. To this end, levels of Erk1, Erk2, and phosphor-Erk1/2 were characterized using a whole cell ELISA. Mek/Erk activity is associated with the proliferative activities of FGF2 through FGFR1 (4,5). Addition of FGF2 and heparin had no significant effect on Erk1 or Erk2 levels for any of the time points examined (FIG. 11). FGF2 caused a 2-3 fold upregulation of phospho-Erk1/2 at all time points investigated. Heparin, however, caused a ˜50% reduction in phosphor-Erk1/2 levels.

To further examine the mechanism by which heparin induces a cell mediated response, PC-3 cells were treated with heparin along with kinase inhibitors. Inhibition of phosphoinositol 3-kinase, which is downstream of FGFRs (6), with LY294002 abrogated the inhibitory capacity of heparin. Mek/Erk inhibition of PD98059 and Mek inhibition with U0126 also eliminate the ability of heparin to inhibit PC-3 growth. SB203580, which inhibits P38, however, did not eliminate the effect of heparin, although the inhibitory capacity was significantly reduced (p>0.002).

Heparin Inhibits PC-3 Growth in Vivo

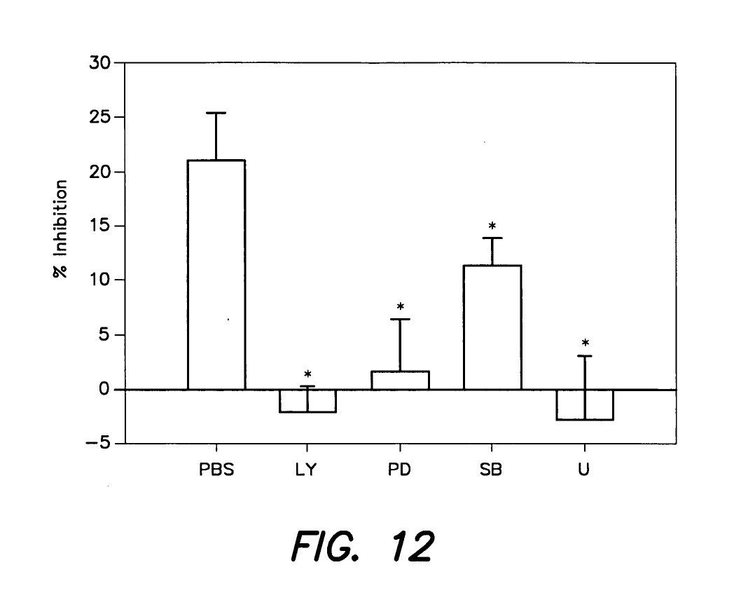

The capacity of heparin to inhibit PC-3 tumor growth was examined in vivo. PC-3 tumors in mice flanks were injected intratumorally with 5, 50, or 500 ng heparin or an equivalent volume of vehicle (FIG. 12). Injections of 50 ng and 500 ng heparin showed significant retardation of tumor growth compared to vehicle alone at all days examined (p<0.009). Treatment with 5 ng heparin retarded tumor growth through day 6 (p<0.02), but tumor size (volume) was no longer significantly lower than vehicle on day 8. Serum was collected from experimental animals after completion of the experiment. None of the heparin treatments produced a significant difference in factor X or factor II activity. Tumors were excised and stained using hematoxylin and eosin (H+E). Sections showed evidence of poor differentiation, invasion, tumor cells in lymph nodes, and tumor cells in vasculature. Treated and untreated cases were not overtly different in amounts of necrosis, invasion or malignancy.

### REFERENCES FOR EXAMPLE 2

1. Natke B, Venkataraman G, Nugent M A, Sasisekharan R. Heparinase treatment of bovine smooth muscle cells inhbits fibroblast growth factor-2 binding to fibroblast growth factor receptor but not FGF-2 mediated cellular proliferation. Angiogenesis 2000;3:249-57.
2. Lersch C, Gericke D, Classen M. Efficacy of low-molecular-weight heparin and unfractionated heparin to prevent adhesion of human prostate and bladder carcinoma and melanoma cells to bovine endothelial monolayers. An in vitro study and review of the literature. Urol Int 1996;56:230-3.
3. Nakamoto T, Chang C S, Li A K, Chodak G W. Basic fibroblast growth factor in human prostate cancer cells. Cancer Res 1992;52:571-7.
4. Bayatti N, Engele J. Cyclic AMP modulates the response of central nervous system glia to fibroblast growth factor-2 by redirecting signalling pathways. J Neurochem 2001;78:972-80.
5. Berry D, Shriver Z, Natke B, Kwan C, Venkataraman G, Sasisekharan R. Heparan sulfate glycosaminoglycan derived from endothelial cells and smotth muscle cells differentially modulate Fibroblast Growth Factor-2 biological activity through Fibroblast Growth Factor Receptor-1. Biochem J 2003;372:1-9.
6. Mochizuki Y, Tsuda S, Kanetake H, Kanda S. Negative regulation of urokinase-type plasminogen activator production through FGF-2-mediated activation of phosphoinositide 3-kinase. Oncogene 2002;21:7027-33.

### Example 3

### Heparinases Inhibit Burkitt's Lymphoma Growth

Materials and Methods

Proteins and Reagents

Porcine intestinal mucosa heparin was from Celsus Laboratories (Columbus, Ohio). FBS was from Hyclone (Logan, Utah). MEM, DMEM, RPMI-1640, PBS, HEPES, sodium pyruvate, sodium bicarbonate, L-glutamine, and penicillin/streptomycin were obtained from GibcoBRL (Gaithersberg, Md.). Transferrin, insulin, oxaloacetic acid, and β-mercaptoethanol were obtained from Sigma (St. Louis, Mo.). NCTC medium 109, B16-F10, Daudi, NFS-1.0 C-1, and J.CaM1.6 cells were from American Type Culture Collection (Manassas, Va.).

Polymer-Heparin Conjugate Synthesis

A single polymer, “A5,” was selected for this study based on previous screens of a 140-polymer library which identified an optimized PAE-heparin conjugate that elicited a maximal cellular mediated response. A5 was prepared as described (22). To form A5-heparin conjugates, A5 was dissolved with vortexing in 25 mM sodium acetate, pH 5.0, and mixed with heparin in 25 mM sodium acetate to produce a 20:1 polymer:heparin ratio (w/w). The mixture was shaken for 30 minutes at room temperature. The complexes were stored at 4° C. until use, which was no greater than 3 hours after conjugation.

Cell Culture

B16-F10 mouse melanoma cells were maintained in MEM supplemented with 10% FBS. B16-F10 cells were grown in 75 cm² flasks at 37° C. in a 5% CO₂ humidified incubator and passaged 2-3 times a week, at confluence. Daudi human Burkitt's lymphoma cells, J.CaM1.6 human M1 leukemia cells, and NFS-1.0 C-1 mouse follicular lymphoma cells were maintained as suspension cultures and grown in 75 cm² flasks at 37° C. in a 5% CO₂ humidified incubator, and were passaged 1:10 by dilution three times a week. Daudi were grown in propagation media composed of RPMI-1640 supplemented with 10% FBS. J.CaM1.6 cells were grown in RPMI-1640 supplemented with 1.5 g/L sodium bicarbonate, 10 mM HEPES, 1.0 mM sodium pyruvate, and 10% FBS. NFS-1.0 C-1 cells were maintained in DMEM supplemented 10 mM HEPES, 1 mM oxaloacetic acid, 0.2 U/ml insulin, 0.5 mM sodium pyruvate, 0.05 mM β-mercaptoethanol, 2 μg/ml transferrin, 10% NCTC medium 109, and 10% FBS. All media was supplemented with 100 μg/ml penicillin, 100 U/ml streptomycin, and 500 μg/ml L-glutamine.

Conjugate Internalization

Fluorescein-conjugated heparin (Molecular Probes, Eugene, Oreg.) was complexed with A5 as described for unconjugated heparin. To determine whether A5 conjugation enabled internalization, confluent Daudi cultures were washed in FBS-deficient media three times, and resuspended in 10 ml FBS-deficient media. Cell concentration was determined with an electronic cell counter and the solution was diluted to 5×10⁴ cells/ml. Wells of 24-well plates were supplemented with 1 ml cell suspension. Four wells were treated with A5-heparin conjugates formulated at a 20:1 (w/w) ratio sufficient to yield a final heparin concentration of 1 μg/ml. Four wells were treated with an equivalent amount of polymer alone, four wells with an equivalent amount of unconjugated fluorescein-labeled heparin, and four wells with PBS. Cells were incubated for 24 hours at 37° C., 5% CO2, and visualized with fluorescence microscopy. Digital images were visualized using Scion Image and processed using Adobe Illustrator 10.0 and Adobe Photoshop 7.0.

Whole Cell Proliferation Assay

B16-F10 cells were grown until confluent, washed with PBS, treated with 3 ml trypsin-EDTA per 75 cm² tissue culture flask at 37° C. for 3-5 min, pelleted, and resuspended in FBS-deficient media. Cell concentration was determined with an electronic cell counter. The suspension was diluted to 5×10⁴ cells/ml and cells were seeded in 24-well plates at 1 ml/well. The plates were incubated for 24 hours at 37° C., 5% CO₂, washed with PBS, and resuspended in media supplemented with 0.1% FBS. Cells were treated with PBS, heparin, A5, or A5-heparin conjugate, added in 10 μl quantities, to yield a polymer concentration of 20 μg/ml and a heparin concentration of 1 μg/ml. Cells were incubated at 37° C., 5% CO₂ for 72 hours, treated with 500 μl trypsin-EDTA per well for 5 minutes, and 400 μl was used to determine the cell number with an electronic cell counter. Data were normalized as a percent change relative to the PBS-treated control.

For proliferation assays using Daudi cells, cells were collected from 75 cm² flasks, washed three times with FBS-deficient media or proliferation media, and resuspended into 10 ml of the same media. Cells were diluted to 1×10⁵ cells/ml based on the reading of an electronic cell counter, and plated 1 ml/well in 24-well plates. Wells were treated with PBS, heparin, A5, or A5-heparin conjugate in 10 μl volumes, and incubated for 72 hours at 37° C., 5% CO₂. A5-heparin conjugates were additionally supplemented with 50 μM LY294002, 20 μM PD98059, or 1 μM SB203580, 50 mM sodium chlorate, or 10 ng/ml FGF2, as appropriate. Whole cell number was converted to a percent growth relative to PBS treatment. Proliferation assays on NFS-1.0 C-1 and J.CaM1.6 cells were performed as described for Daudi cells.

To probe the role of sulfation patterns and fine structure in inducing the effects of A5-heparin conjugates, heparin was partially digested with heparinases prior to conjugation. Heparin was diluted to 20 μg/ml in PBS and incubated with 5 mU/ml hepI or hepIII or an equivalent volume of PBS for 30 minutes. Digestion was confirmed by UV spectroscopy at 232 nm. Digested heparin was subsequently conjugated with A5 as described. A5 binding to the heparin fragments was confirmed using an Azure A competition assay as previously described. Daudi cells, plated as described at 1×10⁵ cells/ml in 24-well plates, 1 ml/well, were treated with conjugates at a heparin concentration of 1 μg/ml or an equivalent volume of PBS. After incubating for 72 hours at 37° C., 5% CO₂, the resultant whole cell count was determined by electronic cell counter, and data were converted to a percent growth relative to Daudi treated with PBS alone.

To examine the effects of protamine sulfate and heparinases on Daudi growth, cells were collected from 75 cm² flasks, pelletted, and resuspended into 10 ml of propagation media. Cells were diluted to 1×10⁵ cells/ml based on the reading of an electronic cell counter, and plated 1 ml/well in 24-well plates. Protamine sulfate was added between 1 and 100,000 ng/ml. HepI and hepIII were added between 0.5 and 500 μU/ml for 24, 48, or 72 hours. Whole cell counts from heparinase assays were converted to percent reduction in whole cell number relative to untreated.

Spectrophotometric Assays

Daudi cells were grown to confluence in 75 cm² plates. Cells were washed three times in FBS-deficient media and resuspended in 10 ml FBS-deficient media. The cell suspension was diluted as appropriate based on the reading of an electronic cell counter and cells were plated in 96-well plates.

The MTS proliferation assay (Promega, Madison, Wis.), the lactic acid dehydrogenase (LDH) cytotoxicity assay (Roche, Basel Switzerland) and the caspase-3/-7 apoptosis assay (Roche) were performed as per manufactures' instructions, and the results were determined using a spectrophotometric plate reader. MTS data were normalized as a percent change relative to PBS-treated cells. LDH data were normalized as the percent change of that induced by the positive control (Triton-X) relative to the negative control (PBS). Caspase-3/-7 data were similarly normalized as the percent reduction of that induced by the positive control, camptothecin relative to the negative control (PBS).

Results

Burkitt's lymphoma (BL) is often associated with the Epstein-Barr virus (EBV) and related proteins. BL is a highly malignant B-cell tumor characterized by a chromosomal translocation that causes constitutive activation of c-myc through the juxtaposition with immunoglobulin loci (1). A translocation to the immunoglobulin (Ig) H enhancer, t(8:14); the Igκ locus, t(2;8); or the Igγ locus, t(8;22); is critical in the initiation of BL, leading to a reduction in apoptotic activity as well as ubiquitin conjugates (2,3). Gene products of the Epstein-Barr virus (EBV) are involved in promoting the tumorigenicity of BL, facilitating the deregulation of c-myc (4,5).

The EBV oncoprotein, latent membrane protein (LMP) 1, for example, has been associated with the induction of factors promoting tumor progression, including the extracellular release of FGF2 from epithelial cells (6). The expression of FGF2, which binds HSGAGs, whose activity is regulated by HSGAGs, has been additionally associated with a worse prognosis in patients with BL (7,8). Other HSGAG-binding proteins and cell surface heparan sulfate proteoglycans can promote EBV gene expression as well as apoptotic cell death. Syndecan-1, a cell surface heparan sulfate proteoglycan (HSPG), has been associated with the onset and proliferation of lymphoma (9). Similarly, phorbol-12-myristate-13-acetate (PMA), which promotes the shedding of syndecan-1 and -4 (10), induces the lytic cycle of EBV genes as well as apoptosis (11). Tumor growth factor (TGF)-β, whose activities are also modulated by HSGAGs, also activates the lytic cycle of EBV in addition to cellular apoptosis (12-14). HSGAGs may be utilized, therefore, to inhibit BL proliferation through a number of important pathways.

In the absence of serum, free heparin inhibited cell growth>30%, while internalization of heparin using PAEs promoted proliferation up to 58%. The growth promoting affects are phosphoinositol-3 kinase (PI3K)-, Erk/Mek- and cell surface HSGAG-dependent, and are minimized in the presence of serum. These findings confirmed that HSGAGs could be harnessed to influence BL cell growth. In the presence of serum, protamine sulfate, heparinase I, and heparinase III inhibited proliferation with the greatest effect induced by heparinase I. These results demonstrate that cell surface HSGAGs are a potential therapeutic target in BL. Furthermore, the ability of HSGAGs to influence cell growth is dependent not only on structure, but also on HSGAG location.

A5-Heparin Conjugates Induce B16-F10 Cell Death

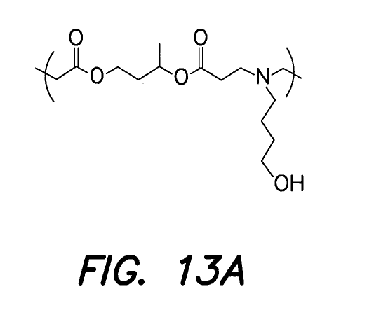

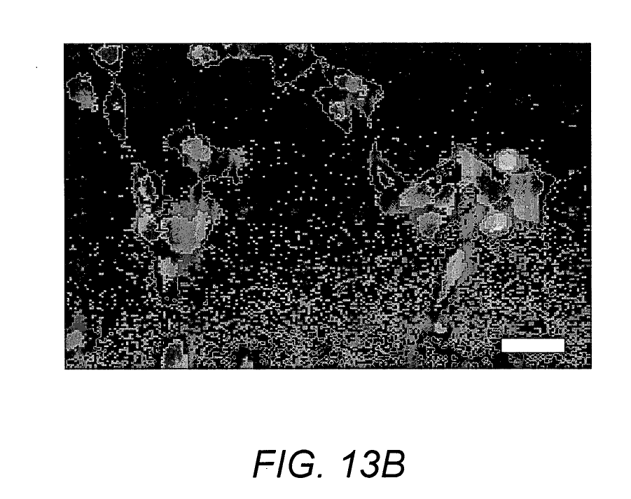

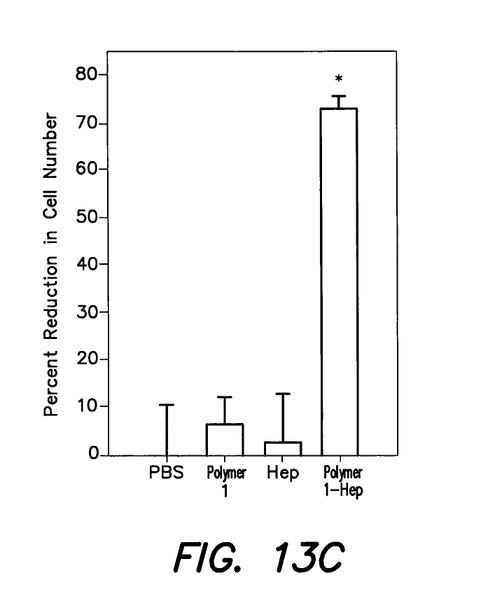

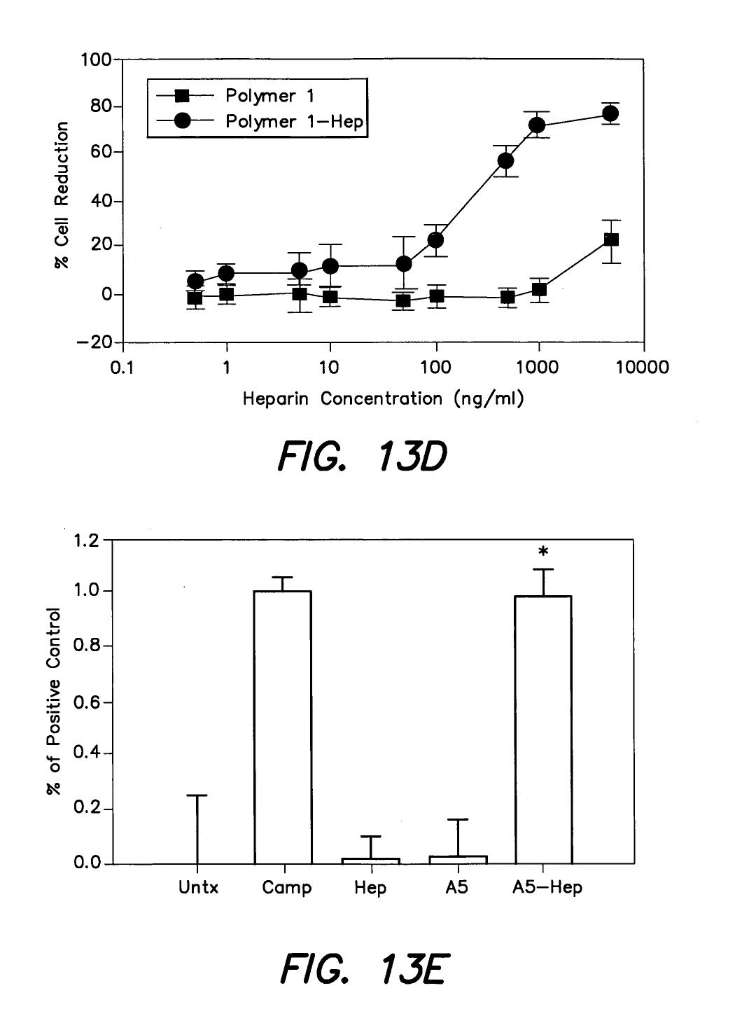

PAEs enable the internalization of DNA and heparin, presumably by creating positively charged complexes less than 200 nm in diameter, promoting endocytosis (22-24). It has been found, however, that the complexes can have a diameter larger than 200 nm. The polymer used herein, A5 (FIG. 13A), has been demonstrated to bind heparin, promote its uptake into cancer cells such as B16-F10 mouse melanoma cells, and reduce the proliferative capacity of these cells (FIGS. 13B and 13C). Treatment of B16-F10 cells with A5-heparin formulated at a 20:1 ratio (w/w) produced a 73.1±2.8% reduction in whole cell number with a heparin concentration of 1 μg/ml. The equivalent concentration of polymer alone (20 μg/ml) did not significantly alter the proliferation of B16-F10 cells (6.1±5.9% inhibition; p>0.51). Unconjugated heparin at 1 μg/ml also had no significant effect on whole cell count, causing a 2.4±10.3% reduction in whole cell number (p>0.67). Higher concentrations of polymer alone directly inhibited whole cell proliferation (FIG. 13D). Conjugates were, therefore, used at a heparin concentration of 1 μg/ml. A5-heparin conjugates activate caspase-3 and -7, consistent with the induction of apoptotic cell death (FIG. 13E).

A5-Heparin Conjugates Promote BL Cell Proliferation

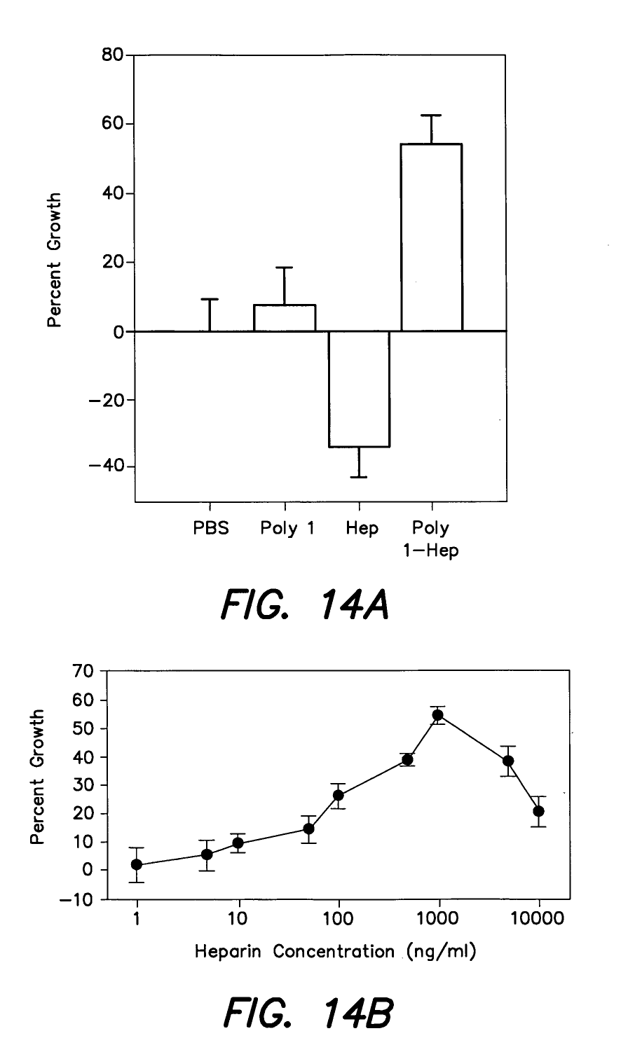

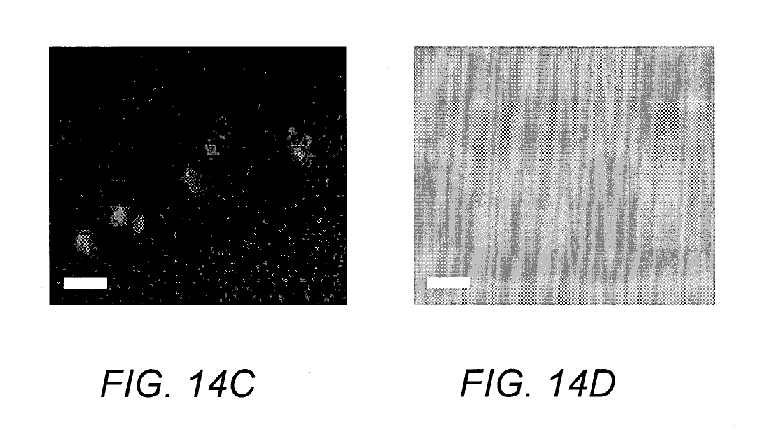

Since HSGAG binding proteins and HSPGs are intimately connected with both the lytic cycle of EBV genes and BL cell apoptosis (6,9,11), whether free HSGAGs and A5-heparin conjugates could influence the proliferation of BL cells using Daudi cells, a BL cell line that contains the EBV genome and a subset of latent proteins (20,21) was investigated. Daudi cells were treated with A5 (20 μg/ml), heparin (1 μg/ml) or A5-heparin (20:1 ratio, w/w, 1 μg/ml heparin) in the absence of serum. Heparin alone caused a 33.8±9.1% reduction in whole cell number (p<7×10⁻⁴), while A5-heparin induced a 58.2±8.6% increase in proliferation (p<2×10⁻⁵; FIG. 14A). A5 alone had no significant effect (p>0.38). A dose-response curve for A5-heparin treatment of Daudi cells was subsequently generated (FIG. 14B). The proliferative capacity of A5-heparin was dose-dependent with a maximal proliferative capacity of 55.2±2.9% observed at 1 μg/ml heparin concentration, 20 mg/ml A5. Notably, administration of A5-heparin at concentrations greater than 1 μg/ml produced less of a proliferative response.

The ability of heparin and A5-heparin to augment proliferation was additionally examined with NFS 1.0 C-1 mouse follicular lymphoma cells and J.CaM1.6 human M1 leukemia cells. No significant effect was observed with heparin, A5, or A5-heparin, for either cell type. The differential effects of A5-heparin to reduce cell number was consistent with findings that demonstrated that the efficaciousness of A5-heparin is cell specific. Correspondingly, while cancer cells typically have a greater magnitude of response to A5-heparin than non-cancer cells, some cancer cell lines are not susceptible to its effects.

While the distinct cellular mediated effects of free heparin and A5-heparin are consistent with the polymer imparting novel function, it was confirmed that in the Daudi cell line, the conjugation with A5 facilitated the internalization of heparin. To confirm that A5-heparin induced its distinct proliferative effects through the internalization of heparin, fluorescein-conjugated heparin was complexed with A5 and applied to Daudi cells. Fluorescence microscopy showed substantial co-localization of fluorescence with cells (FIGS. 14C and 14D). The degree of co-localization was much greater than that observed with free fluorescein-conjugated heparin, consistent with internalization. Even at higher magnification, the conjugates did not show localization to regions with the cells.

A5-Heparin Conjugates Activate Both Proliferative and Apoptotic Pathways

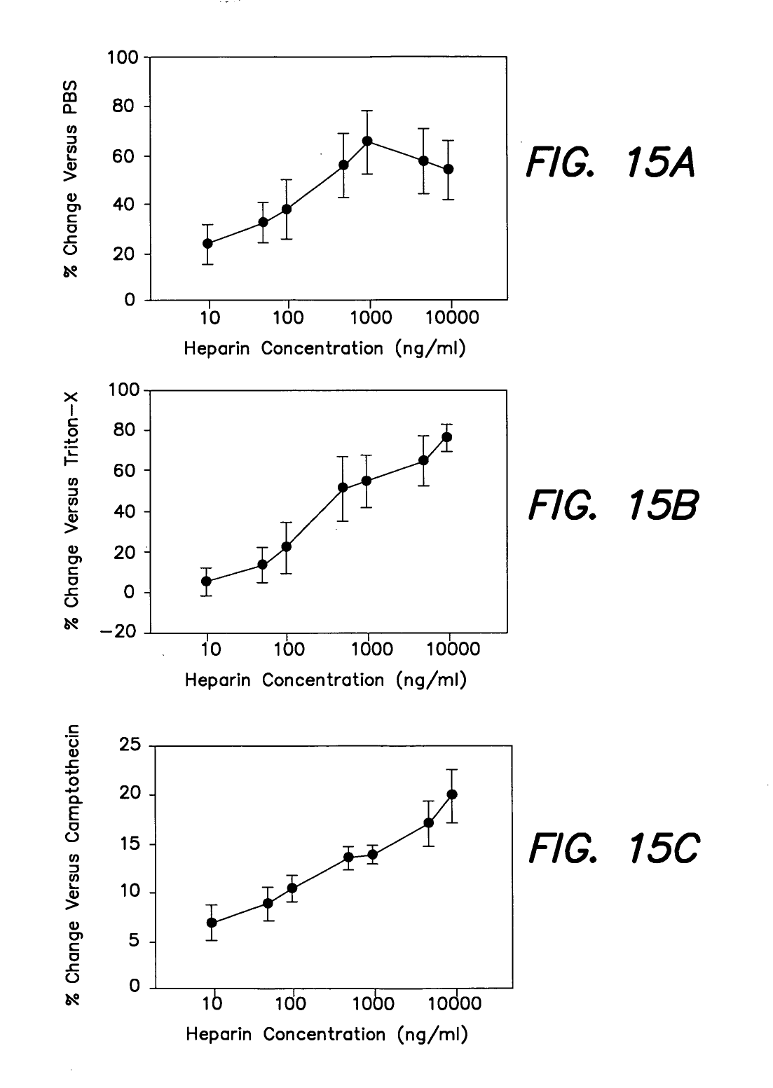

While heparin alone promoted a ˜30% growth inhibition, the magnitude of effect was greater with the A5-heparin conjugate, and therefore subsequent studies performed herein focused on the A5-heparin conjugate. To confirm the observed increase in whole cell number and to probe the mechanism by which A5-heparin induces its proliferative response, a MTS proliferation assay, a LDH cytotoxicity assay, and a caspase-3/-7 apoptosis assay was employed. All three assays demonstrated a dose-dependent response to A5-heparin. The MTS assay, in which a tetrazolium salt was used to detect mitochondrial integrity, produced a response pattern that was similar to that as with whole cell counts (FIG. 15A). A5-heparin administered with a heparin concentration of 1 μg/ml induced the maximal response, 65.4±12.5%, greater than that of the PBS control. At heparin concentrations greater than 1 μg/ml, a progressive decline in response level was observed.

The LDH cytotoxicity assay revealed that A5-heparin promoted LDH release that increased with concentration (FIG. 15B). No plateau was observed over the range examined, up to a heparin concentration of 10 μg/ml. The peak response, at 10 μg/ml heparin, was a cytotoxic response that was 75.41±6.56% of that induced by Triton-X, the positive control. The caspase-3/-7 apoptosis assay similarly revealed increasing responses with increasing concentrations of A5-heparin (FIG. 15C). No plateau concentration was determined. At a heparin concentration of 10 μg/ml, A5-heparin induced an apoptotic response that was 19.83±2.77% of that induced by camptothecin.

Taken together, these results demonstrate that A5-heparin promotes proliferation but also apoptosis. Based on the dose-response curves generated, the signals that support cell proliferation predominate at heparin concentrations of 1 μg/ml and below. As concentration increased, however, so too did apoptotic activity, as measured by caspase-3 and -7 activity. The dual activation of two sets of processes supports the shape of the dose-response curves generated by whole-cell counts and the MTS assay. Critical modulators and transcripts in BL require careful regulation to promote growth and avoid apoptosis. TGF-β, anti-Ig, and PMA, for example each promote the expression of EBV genes but also cell apoptosis (11). In BL, the apoptotic signal may be overcome, however, by the expression anti-apoptotic factors including BHRF-1, a Bc12 homolog that associates with Bax and Bak (25,26). Similarly, A5-heparin may activate multiple pathways, the concentration of which defines the observed phenotype. In B16-F10 cells, polymer-heparin conjugates promote apoptosis by rapid incorporation and interactions with transcription factors that alter their normal activities. However, this includes the transcription factor Sp-1, the levels of which are upregulated in both the cytoplasm and the nucleus. Sp-1 is induced downstream of the EBV-protein LMP1, and involved in the activation of the matrix metalloproteinase 9 promoter, supporting cell viability (27). As such, A5-heparin may promote processes that support both proliferation and apoptosis.

AS-Heparin Mediated Proliferation is PI3K and Erk/Mek Dependent

The Erk/Mek pathway was investigated as it is associated with Sp-1 activity downstream of growth factors (28,29). PI3K was additionally investigated as FGF2, also associated with LMP1 (6), induces the phosphorylation of this kinase as well as Erk/Mek activation (30,31).

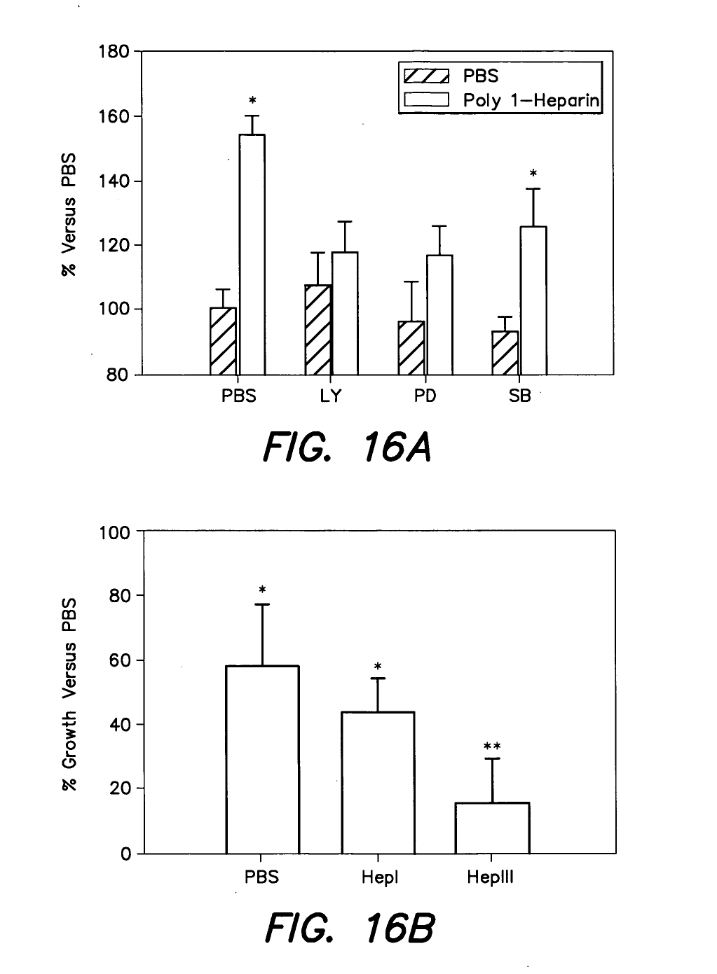

Daudi cells in FBS-deficient media were treated with 50 μM LY294002, 20 μM PD98059, or μM SB203580 as well as PBS or A5-heparin (20:1, w/w, 1 μg/ml heparin concentration). While neither inhibition of PI3K with LY294002 (p>0.39) nor inhibition of Erk and Mek with PD98059 (p>0.64) had a direct effect on Daudi whole cell number, inhibition of p38 with SB203580 caused a significant reduction (p<0.02). Application of A5-heparin in SB203580 maintained the proliferative response evident in the absence of kinase inhibitors (FIG. 16A). The response in the presence of SB203580 was 35.77±5.34% greater than the kinase inhibitor alone (p<0.005). A5-heparin, however, failed to induce a significant proliferative response in the presence of either LY294002 or PD98059 relative to the kinase inhibitor alone. These results suggest that the proliferative response of A5-heparin is dependent on Erk/Mek and PI3K.

To confirm that the effects of A5-heparin were dependent on the heparin component, heparin was treated with hepI, hepIII or PBS prior to conjugation with A5. Conjugates were then formed and applied to cells in the same method as for full length heparin. Heparinase treatment did not prevent A5 binding, as confirmed using an Azure A competition assay. Digestion of heparin with hepI reduced the proliferative capacity of the A5-heparin conjugate to 44.1±10.4% (FIG. 16B), not significantly less than PBS treated heparin (p>0.13). Digestion with hepIII, however, produced a proliferative response (15.5±14.0%) less than that of PBS treated heparin (p<5×10⁻⁷), and not significantly greater than PBS treatment of Daudi cells alone (p>0.11). These results demonstrate that the observed phenotype requires the HSGAG component, and furthermore, the structure of the HSGAG is important in defining the biological response.

Cell Surface HSGAGs are Necessary for A5-Heparin Mediated Proliferation

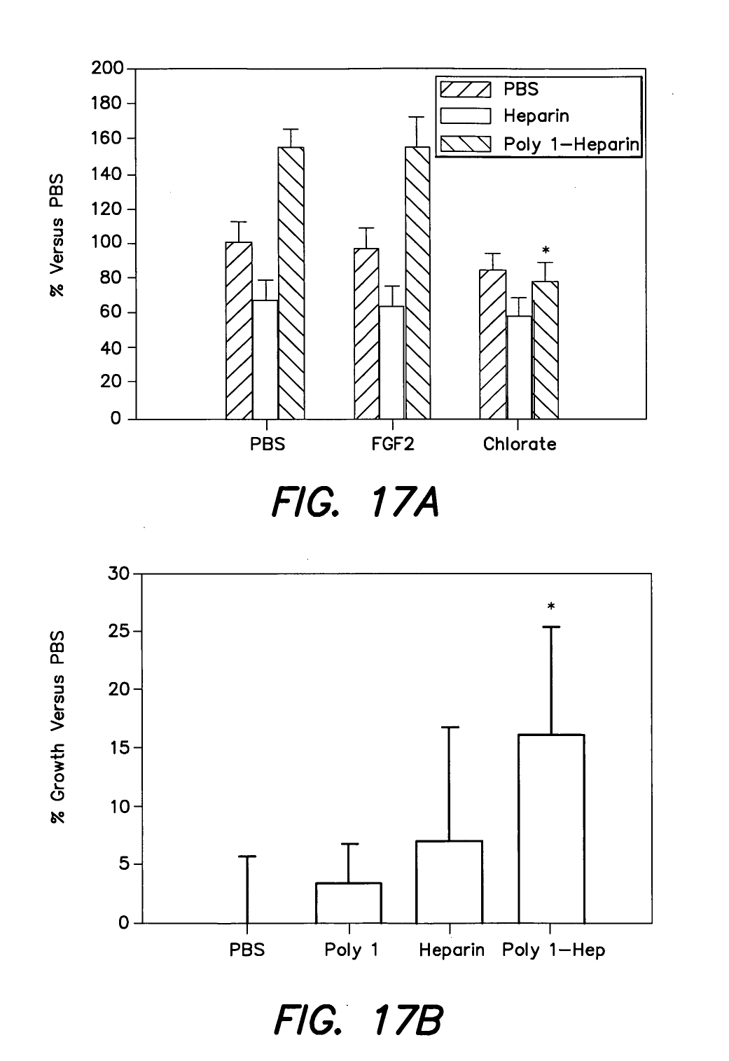

To further probe the mechanism by which A5-heparin promotes proliferation, whether FGF2 and cell surface HSGAGs were important was examined. Administration of FGF2 did not affect Daudi proliferation, the capacity of heparin to inhibit it, or A5-heparin to promote it (p>0.71). Treatment with sodium chlorate, which prevents heparan sulfate biosynthesis (32,33), did not affect the resultant whole cell count or heparin-mediated inhibition of proliferation, but did, however, abrogate the capacity of A5-heparin to induce a proliferative response (FIG. 17A). The combination of A5-heparin and sodium chlorate elicited a response not significantly different from sodium chlorate alone (p>0.52), but less than the effect of A5-heparin treatment in the absence of sodium chlorate (p<2×10⁻⁶). Serum also reduced the ability of A5-heparin to promote proliferation, and similarly eliminated the growth inhibitory capacity of free heparin (FIG. 17B).

While FGF2 did not affect A5-heparin proliferation, other FGF family members and other growth factor families may influence the proliferative response. Nonetheless, cell surface HSGAGs were important to A5-heparin proliferation. The GAG component of HSPGs, rather than-the protein core itself, has been implicated in PI3K- and Erk/Mrk-mediated responses (34,35). Cell surface associated HSPGs, including syndecan-1 and syndecan-4, are important in BL proliferation (9,10). The importance of cell surface HSGAGs in A5-heparin effects suggests that the GAG component of HSGAGs may confer the biological properties observed.

HSGA Gs can be Harnessed to Inhibit BL Proliferation

The information about how A5-heparin may promote proliferation was used to develop a way to inhibit BL growth in the presence of serum. High concentrations of A5-heparin may induce substantial apoptosis, but A5 alone does have cytotoxic effects at high concentrations. The important nature of cell surface HSGAGs suggested that they may be a viable target to influence BL growth.

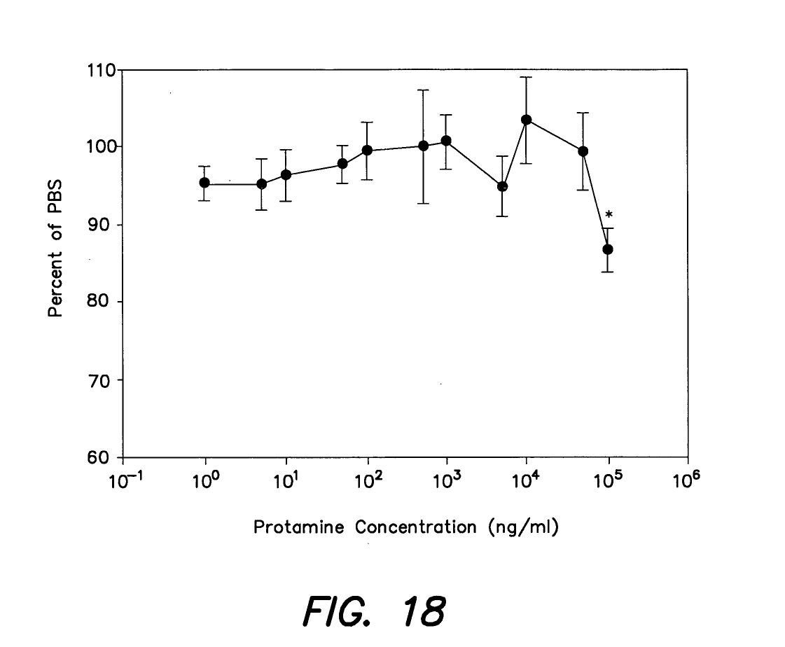

To this end, Daudi cells in media supplemented with 10% FBS were treated with various concentrations of protamine sulfate, a protein with known anti-heparin activities that counteracts the effects of heparin by interfering with protein binding rather than promoting its degradation (36,37). Application of protamine sulfate had no effect at concentrations less than 1×10⁵ ng/ml (FIG. 18). At a concentration of 1×10⁵ ng/ml, however, protamine sulfate induced a 12.9±2.8% reduction in whole cell number (p<3×10⁻⁶).

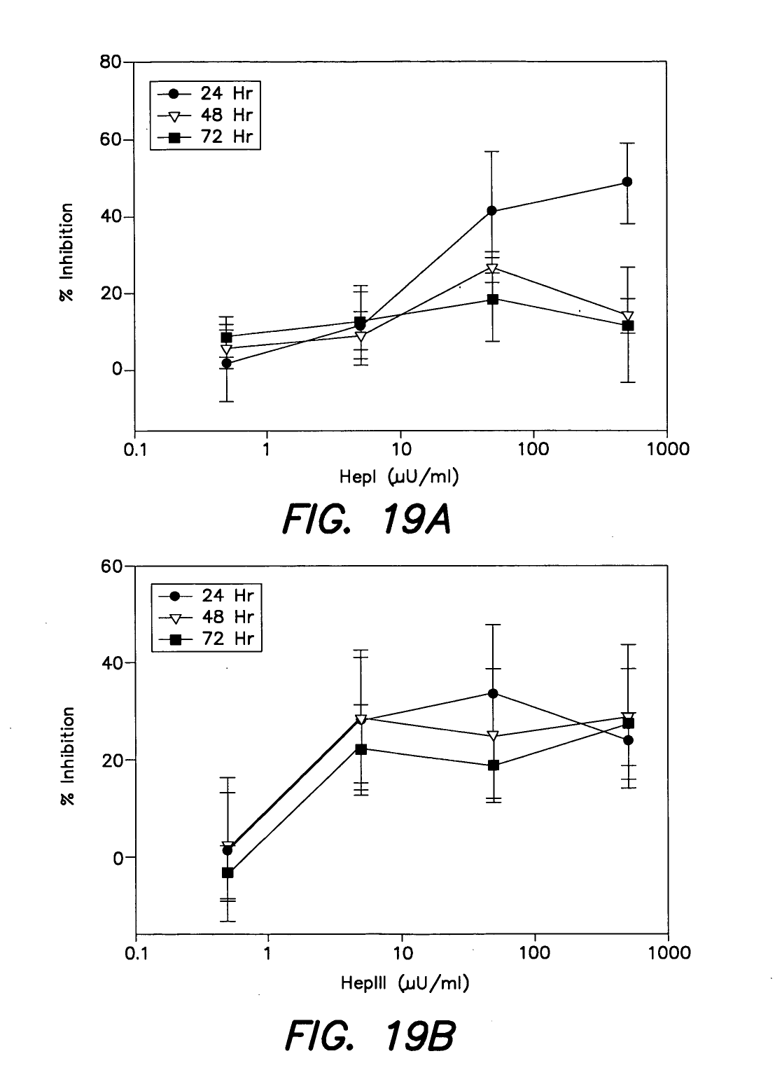

Since the anti-proliferative affects of protamine sulfate were only at high dose, whether digestion with heparinases, which differentially digest HSGAGs based on the distribution of sulfate groups, could inhibit Daudi proliferation was explored. HepI and hepIII were applied to Daudi cells in media supplemented with 10% FBS over a range of concentrations and incubated for 24, 48, or 72 hours. Both hepI and hepIII inhibited proliferation in a dose-dependent manner (FIG. 19). HepIII treatment promoted ˜30% inhibition at concentrations between 5 and 500 μU/ml. The time of incubation did not alter the inhibitory capacity of hepIII, as 24, 48, and 72 hr incubations had the same potency and efficaciousness. The effect of hepI treatment, however, was time-dependent. Incubations for 24 hrs were more efficacious than those for 48 or 72 hrs at concentrations of 50 μU/ml (p<3×10⁻⁵) and 500 μU/ml (p<6×10⁻⁵). Furthermore, the 49.7±10.4% inhibition obtained with 500 μU/ml was significantly greater than the maximal inhibitory effect, 33.7±14.5%, obtained with hepIII (p<0.05).

In this study, the ability of HSGAGs to influence the proliferative capacity of BL cells was examined. Daudi cells, which contain the full EBV genome and express a restricted set of EBV latent genes (20,21), were used as an in vitro model of BL. Exogenous heparin was examined as well as heparin internalized using PAEs, a class of cationic polymers that binds to and enables the internalization of both DNA and heparin (22-24). In the absence of serum, internalized heparin was found to strongly promote BL growth while heparin slightly inhibits it. Internalized heparin-mediated proliferation was investigated as its response was more robust than that of free heparin, and the ability of internalized heparin to promote proliferation was not observed in other cell types. The proliferative effect of internalized heparin was dependent on cell surface HSGAGs, PI3K, and Erk/Mek. It was then determined if HSGAGs could be altered to inhibit BL proliferation in the presence of serum, where the effects of free heparin and internalized heparin were mitigated. HepI was found to inhibit Daudi proliferation ˜50%. These results demonstrate the importance of HSGAGs in the proliferation of BL cells and suggest that HSGAGs and processes that they affect are potential therapeutic targets. Treatment of BL cells with hepI provides an efficacious method to inhibit cell growth. The ability of hepIII treatment to also inhibit cell growth is consistent with viability of HSGAGs as a target to influence BL growth. However, efficient growth inhibition requires digestion of HSGAGs rather than binding interference.

HSGAGs can be harnessed in multiple ways to differentially influence cancer cell growth, though the specific effects may be cell-specific. Not only can manipulation of HSGAG content to contain bioactive regions, such as by enzymatic digestion, directly influence the capacity to invoke a cellular response, but also, controlled localization of HSGAGs enables the regulation of the type of response elicited. Therefore, manipulation of both content and location may serve to optimize the efficacy of HSGAGs as therapeutic agents.

### REFERENCES FOR EXAMPLE 3

1. Klein G. Specific chromosomal translocations and the genesis of B-cell-derived tumors in mice and men. Cell 1983;32:311-5.
2. Klein G. The role of gene dosage and genetic transpositions in carcinogenesis. Nature 1981;294:313-8.
3. Gavioli R, Frisan T, Vertuani S, Bornkamm G W, Masucci M G. c-myc overexpression activates alternative pathways for intracellular proteolysis in lymphoma cells. Nat Cell Biol 2001;3:283-8.
4. Ruf I K, Rhyne P W, Yang H, Borza C M, Hutt-Fletcher L M, Cleveland J L, et al. Epstein-barr virus regulates c-MYC, apoptosis, and tumorigenicity in Burkitt lymphoma. Mol Cell Biol 1999;19:1651-60.
5. Ruf I K, Rhyne P W, Yang H, Borza C M, Hutt-Fletcher L M, Cleveland J L, et al. EBV regulates c-MYC, apoptosis, and tumorigenicity in Burkitt's lymphoma. Curr Top Microbiol Immunol 2001;258:153-60.
6. Wakisaka N, Murono S, Yoshizaki T, Furukawa M, Pagano J S. Epstein-barr virus latent membrane protein 1 induces and causes release of fibroblast growth factor-2. Cancer Res 2002;62:6337-44.
7. Ho C L, Sheu L F, Li C Y. Immunohistochemical expression of basic fibroblast growth factor, vascular endothelial growth factor, and their receptors in stage IV non-Hodgkin lymphoma. Appl Immunohistochem Mol Morphol 2002;10:316-21.
8. Padera R, Venkataraman G, Berry D, Godvarti R, Sasisekharan R. FGF-2/fibroblast growth factor receptor/heparin-like glycosaminoglycan interactions: a compensation model for FGF-2 signaling. Faseb J 1999;13:1677-87.
9. Sebestyen A, Totth A, Mihalik R, Szakacs O, Paku S, Kopper L. Syndecan-1-dependent homotypic cell adhesion in HT58 lymphoma cells. Tumour Biol 2000;21:349-57.
10. Fitzgerald M L, Wang Z, Park P W, Murphy G, Bernfield M. Shedding of syndecan-1 and -4 ectodomains is regulated by multiple signaling pathways and mediated by a TIMP-3-sensitive metalloproteinase. J Cell Biol 2000;148:811-24.
11. Inman G J, Binne U K, Parker G A, Farrell P J, Allday M J. Activators of the Epstein-Barr virus lytic program concomitantly induce apoptosis, but lytic gene expression protects from cell death. J Virol 2001;75:2400-10.
12. Chaouchi N, Arvanitakis L, Auffredou M T, Blanchard D A, Vazquez A, Sharma S. Characterization of transforming growth factor-beta 1 induced apoptosis in normal human B cells and lymphoma B cell lines. Oncogene 1995;11:1615-22.
13. Lyon M, Rushton G, Gallagher J T. The interaction of the transforming growth factor-betas with heparin/heparan sulfate is isoform-specific. J Biol Chem 1997;272:18000-6.
14. Inman G J, Allday M J. Apoptosis induced by TGF-beta 1 in Burkitt's lymphoma cells is caspase 8 dependent but is death receptor independent. J Immunol 2000;165:2500-10.
15 Perrimon N, Bernfield M. Specificities of heparan sulphate proteoglycans in developmental processes. Nature 2000;404:725-8.
16. Esko J D, Lindahl U. Molecular diversity of heparan sulfate. J Clin Invest 2001;108:169-73.
17. Blackhall F H, Merry C L, Davies E J, Jayson G C. Heparan sulfate proteoglycans and cancer. Br J Cancer 2001;85:1094-8.
18. Sasisekharan R, Shriver Z, Venkataraman G, Narayanasami U. Roles of heparan-sulphate glycosaminoglycans in cancer. Nat Rev Cancer 2002;2:521-8.
19. Liu D, Shriver Z, Venkataraman G, El Shabrawi Y, Sasisekharan R. Tumor cell surface heparan sulfate as cryptic promoters or inhibitors of tumor growth and metastasis. Proc Natl Acad Sci USA 2002;99:568-73.
20. Klein E, Klein G, Nadkarni J S, Nadkarni J J, Wigzell H, Clifford P. Surface IgM-kappa specificity on a Burkitt lymphoma cell in vivo and in derived culture lines. Cancer Res 1968;28:1300-10.
21. Nanbo A, Inoue K, Adachi-Takasawa K, Takada K. Epstein-Barr virus RNA confers resistance to interferon-alpha-induced apoptosis in Burkitt's lymphoma. Embo J 2002;21:954-65.
22. Lynn D M, Anderson D G, Putnam D, Langer R. Accelerated Discovery of Synthetic Transfection Vectors: Parallel Synthesis and Screening of a Degradable Polymer Library. J. Am. Chem. Soc. 2001;123:8155-6.
24. Lynn D M, Langer R. Degradable Poly(β-amino esters): Synthesis, Characterization, and Self-assembly with plasmid DNA. J. Am. Chem. Soc 2000;122:10761-8.
25. Marshall W L, Yim C, Gustafson E, Graf T, Sage D R, Hanify K, et al. Epstein-Barr virus encodes a novel homolog of the bcl-2 oncogene that inhibits apoptosis and associates with Bax and Bak. J Virol 1999;73:5181-5.
26. Henderson S, Huen D, Rowe M, Dawson C, Johnson G, Rickinson A. Epstein-Barr virus-coded BHRF1 protein, a viral homologue of Bcl-2, protects human B cells from programmed cell death. Proc Natl Acad Sci USA 1993;90:8479-83.
27. Yoshizaki T, Sato H, Furukawa M, Pagano J S. The expression of matrix metalloproteinase 9 is enhanced by Epstein-Barr virus latent membrane protein 1. Proc Natl Acad Sci USA 1998;95:3621-6.
28. Sakuta T, Matsushita K, Yamaguchi N, Oyama T, Motani R, Koga T, et al. Enhanced production of vascular endothelial growth factor by human monocytic cells stimulated with endotoxin through transcription factor SP-1. J Med Microbiol 2001;50:233-7.
29. Ling S, Deng G, Ives H E, Chatterjee K, Rubanyi G M, Komesaroff P A, et al. Estrogen inhibits mechanical strain-induced mitogenesis in human vascular smooth muscle cells via down-regulation of Sp-1. Cardiovasc Res 2001;50:108-14.
30. Ong S H, Guy G R, Hadari Y R, Laks S, Gotoh N, Schlessinger J, et al. FRS2 proteins recruit intracellular signaling pathways by binding to diverse targets on fibroblast growth factor and nerve growth factor receptors. Mol Cell Biol 2000;20:979-89.
31. Ong S H, Hadari Y R, Gotoh N, Guy G R, Schlessinger J, Lax I. Stimulation of phosphatidylinositol 3-kinase by fibroblast growth factor receptors is mediated by coordinated recruitment of multiple docking proteins. Proc Natl Acad Sci USA 2001;98:6074-9.
32. Fannon M, Nugent M A. Basic fibroblast growth factor binds its receptors, is internalized, and stimulates DNA synthesis in Balb/c3T3 cells in the absence of heparan sulfate. J Biol Chem 1996;271:17949-56.
33. Rapraeger A C, Kruffka A, Olwin B B. Requirement of heparin sulfate for bFGF-mediated fibroblast growth and myoblast differentiation. Science 1991;252:1705-8.
34. Berry D, Shriver Z, Natke B, Kwan C, Venkataraman G, Sasisekharan R. Heparan sulfate glycosaminoglycan derived from endothelial cells and smotth muscle cells differentially modulate Fibroblast Growth Factor-2 biological activity through Fibroblast Growth Factor Receptor-1. Biochem J 2003;372:1-9.
35. Qi M, Ikematsu S, Maeda N, Ichihara-Tanaka K, Sakuma S, Noda M, et al. Haptotactic migration induced by midkine. Involvement of protein-tyrosine phosphatase zeta. Mitogen-activated protein kinase, and phosphatidylinositol 3-kinase. J Biol Chem 2001;276:15868-75.
36. Cobel-Geard R J, Hassouna H I. Interaction of protamine sulfate with thrombin. Am J Hematol 1983;14:227-33.
37. Teoh K H, Young E, Bradley C A, Hirsh J. Heparin binding proteins. Contribution to heparin rebound after cardiopulmonary bypass. Circulation 1993;88:11420-5.

### Example 4

### Heparin Inhibits Tumor Growth in Vivo

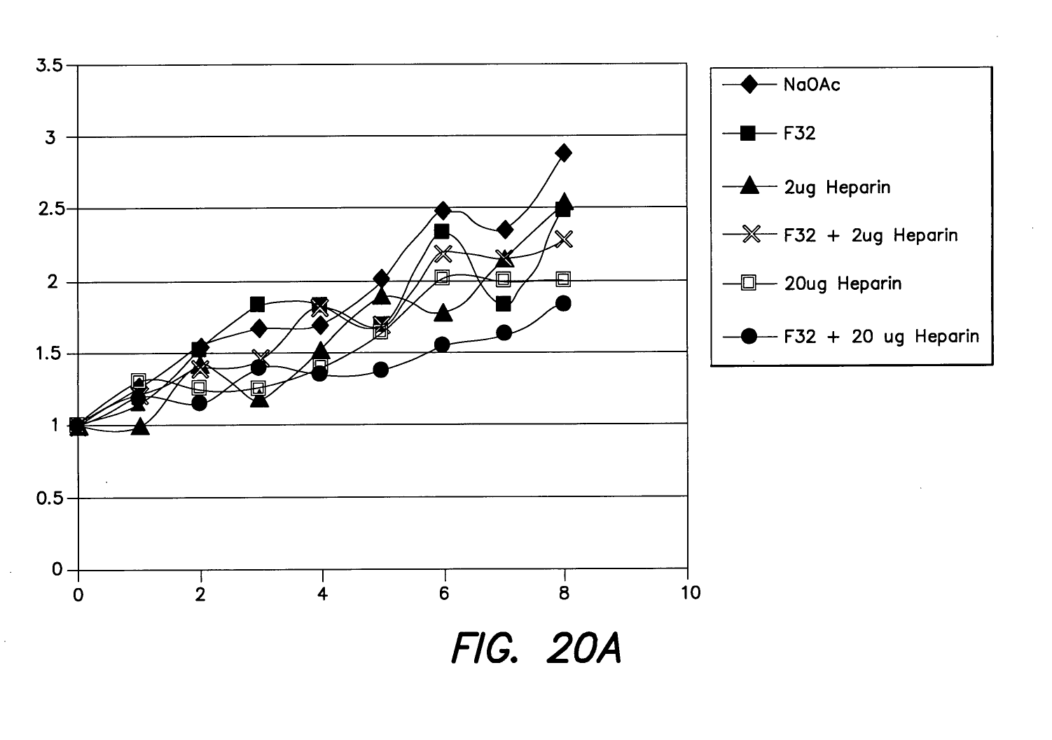

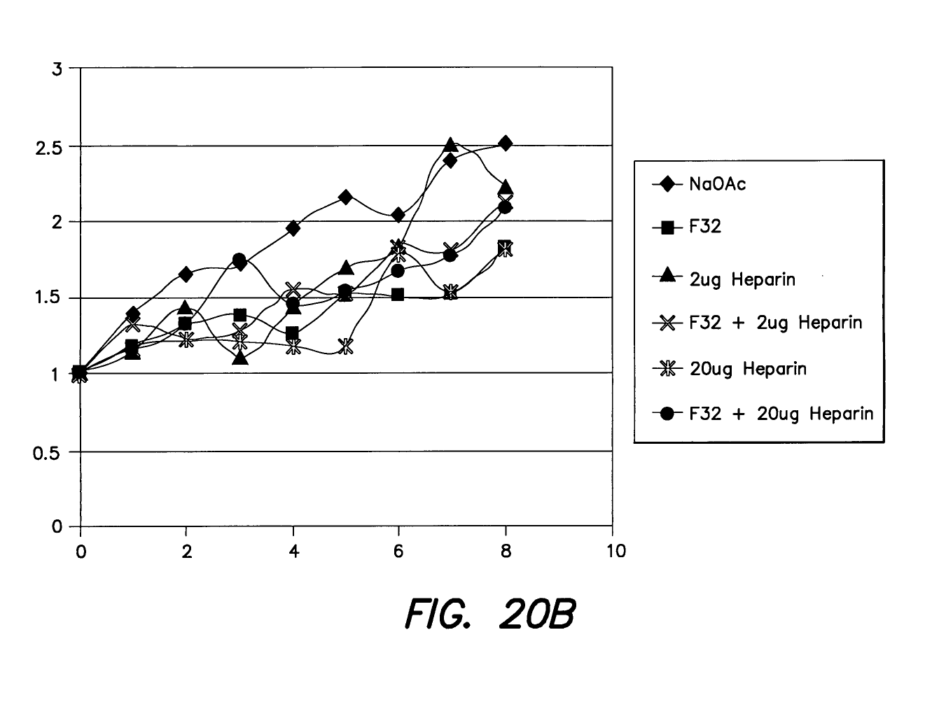

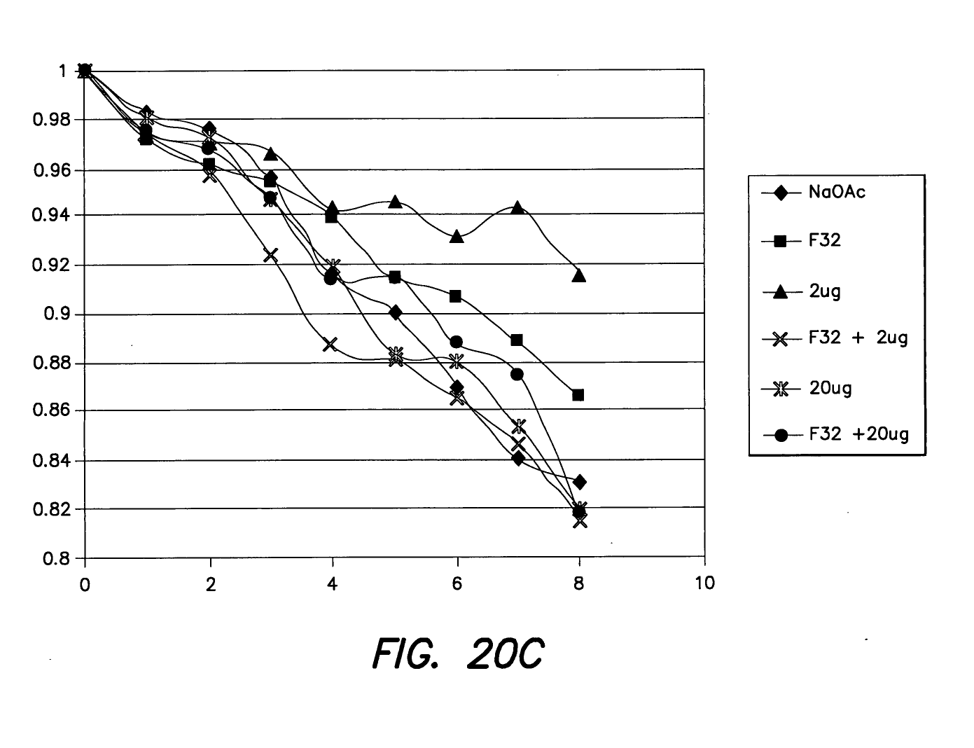

Xenografts were generated in nude (nu/nu.c) Harlan Sprague Dawley mice via the subcutaneous injection of PC-3 human prostatic adenocarcinoma cells into each flank. Tumors were allowed to grow for 1 week until tumor volumes were approximately 50 mm³, after which intratumoral injections were initiated, defined as day 0. Only mice in which tumors on both sides were of similar size were employed herein. Heparin was injected in 2.5 mM sodium acetate in PBS, in a final volume of 100 μl. Three dosing regimens were employed. First, 5 ng, 50 ng, and 500 ng total heparin was injected on day 0 into one tumor of five mice per dose. An equivalent volume of vehicle alone was injected into five mice. Second, 5 mice per dose were treated on day 0 with 0.5 μg, 1 μg, 5 μg, 10 μg and 50 μg heparin. Third, 10 mice per dose were treated with 1 μg and 50 μg heparin on each of days 0-7. Tumor size was measured by caliper throughout the experiment. The results are shown in FIG. 20 and in the following tables:

TABLE 4-1
Treated Side

| | days | | | | | | | | |
|---|---|---|---|---|---|---|---|---|---|
| | 0 | 1 | 2 | 3 | 4 | 5 | 6 | 7 | 8 |
| NaOAC | 1 | 1.389032 | 1.64065 | 1.709986 | 1.937064 | 2.149362 | 2.035342 | 2.387239 | 2.50509 |
| | 0 | 0.276459 | 0.172542 | 0.156175 | 0.384727 | 0.439057 | 0.567072 | 1.030804 | 1.036557 |
| F32 | 1 | 1.167496 | 1.316453 | 1.366189 | 1.255631 | 1.497185 | 1.503879 | 1.519541 | 1.815339 |
| | 0 | 0.169374 | 0.300085 | 0.285086 | 0.389608 | 0.531347 | 0.771103 | 0.621641 | 0.84936 |
| 2 ug | 1 | 1.137318 | 1.429402 | 1.106751 | 1.422238 | 1.681226 | 1.825982 | 2.481106 | 2.209753 |
| Heparin | 0 | 0.112369 | 0.461854 | 0.065419 | 0.206999 | 0.407861 | 0.244815 | 1.28446 | 0.676783 |
| 2 ug + | 1 | 1.319029 | 1.213263 | 1.276857 | 1.539202 | 1.516157 | 1.820794 | 1.802118 | 2.112871 |
| F32 | 0 | 0.251459 | 0.276576 | 0.375466 | 0.472176 | 0.454365 | 0.690408 | 0.808109 | 0.757559 |
| 20 ug | 1 | 1.151435 | 1.221232 | 1.202308 | 1.172504 | 1.167294 | 1.76771 | 1.52675 | 1.800511 |
| Heparin | 0 | 0.233096 | 0.467101 | 0.470937 | 0.209193 | 0.284926 | 0.300699 | 0.201841 | 0.277714 |
| 20 ug + | 1 | 1.139911 | 1.318113 | 1.732305 | 1.439508 | 1.532554 | 1.654692 | 1.756282 | 2.073655 |
| F32 | 0 | 0.18329 | 0.175099 | 0.292883 | 0.266894 | 0.340936 | 0.173914 | 0.315631 | 0.390296 |

TABLE 4-2
Untreated Side

| | days | | | | | | | | |
|---|---|---|---|---|---|---|---|---|---|
| | 0 | 1 | 2 | 3 | 4 | 5 | 6 | 7 | 8 |
| NaOAC | 1 | 0.981974 | 0.975654 | 0.954847 | 0.915459 | 0.899562 | 0.869818 | 0.840376 | 0.830613 |
| | 0 | 0.009928 | 0.012198 | 0.007412 | 0.014707 | 0.024089 | 0.032162 | 0.033886 | 0.030955 |
| F32 | 1 | 0.971045 | 0.960107 | 0.953192 | 0.938786 | 0.914315 | 0.906855 | 0.888716 | 0.865347 |
| | 0 | 0.04058 | 0.046061 | 0.047724 | 0.049353 | 0.060077 | 0.07809 | 0.077548 | 0.075715 |
| 2 ug | 1 | 0.973128 | 0.970269 | 0.965494 | 0.943012 | 0.945547 | 0.931379 | 0.942451 | 0.91496 |
| Heparin | 0 | 0.013726 | 0.012969 | 0.022288 | 0.029753 | 0.013658 | 0.021442 | 0.036079 | 0.034317 |
| 2 ug + | 1 | 0.973916 | 0.955755 | 0.923444 | 0.88796 | 0.88097 | 0.865065 | 0.845513 | 0.814878 |
| F32 | 0 | 0.010082 | 0.007303 | 0.022652 | 0.04694 | 0.041087 | 0.049684 | 0.046035 | 0.035482 |
| 20 ug | 1 | 0.979813 | 0.971274 | 0.946629 | 0.918886 | 0.882669 | 0.880114 | 0.85296 | 0.819403 |
| Heparin | 0 | 0.020799 | 0.012014 | 0.024094 | 0.030986 | 0.025317 | 0.054117 | 0.043804 | 0.038826 |
| 20 ug + | 1 | 0.974648 | 0.96684 | 0.946757 | 0.914097 | 0.914239 | 0.888086 | 0.87419 | 0.818069 |
| F32 | 0 | 0.010113 | 0.014294 | 0.024735 | 0.038359 | 0.041503 | 0.048551 | 0.04777 | 0.046666 |

TABLE 4-3
Weight

| | days | | | | | | | | |
|---|---|---|---|---|---|---|---|---|---|
| | 0 | 1 | 2 | 3 | 4 | 5 | 6 | 7 | 8 |
| NaOAC | 1 | 1.258802 | 1.541353 | 1.660725 | 1.683012 | 2.007327 | 2.464029 | 2.327478 | 2.873181 |
| | 0 | 0.257075 | 0.439144 | 0.347031 | 0.461454 | 0.742836 | 1.048991 | 1.048591 | 1.844803 |
| F32 | 1 | 1.156562 | 1.52404 | 1.822166 | 1.811123 | 1.681895 | 0.319742 | 1.823234 | 2.468849 |
| | 0 | 0.075985 | 0.403799 | 1.053257 | 0.396415 | 0.469519 | 0.97472 | 0.475798 | 1.148256 |
| 2 ug | 1 | 0.99881 | 1.413037 | 1.182572 | 1.523902 | 1.880731 | 1.762346 | 2.131789 | 2.538852 |
| Heparin | 0 | 0.158639 | 0.224852 | 0.340702 | 0.22563 | 0.249825 | 0.120817 | 0.394391 | 0.633993 |
| 2 ug + | 1 | 1.194798 | 1.388365 | 1.445708 | 1.803486 | 1.690598 | 2.161315 | 2.132554 | 2.258513 |
| F32 | 0 | 0.091181 | 0.275051 | 0.299017 | 0.503252 | 0.346431 | 0.476639 | 0.301392 | 0.502294 |
| 20 ug | 1 | 1.301082 | 1.246814 | 1.257158 | 1.397081 | 1.634861 | 1.997466 | 1.985434 | 1.976972 |
| Heparin | 0 | 0.406462 | 0.462459 | 0.33831 | 0.415877 | 0.464075 | 0.464424 | 0.343026 | 0.444383 |
| 20 ug + | 1 | 1.182979 | 1.156487 | 1.377045 | 1.35021 | 1.35921 | 1.535894 | 1.615607 | 1.808821 |
| F32 | 0 | 0.201688 | 0.225049 | 0.15253 | 0.152897 | 0.175832 | 0.282634 | 0.377986 | 0.430939 |

e 5—Polymer-Heparin Conjugate Reverses the Anticoagulant Effect

After completion of the experiments, mice were sacrificed by CO₂ asphyxiation. ˜500 ml blood was collected by cardiac puncture. Blood was centrifuged and plasma extracted. The anticoagulant effect of treatments was assessed by measuring activities of plasma factors Xa and IIa. Plasma was diluted 1:150 in PBS to a final volume of 90 μl, and treated with 600 ng of either chromogenic substrate for factor Xa or for factor IIa (Sigma, St. Louis Mo.) as appropriate in 10 μl PBS. Change in absorbance per second was measured at 405 nm. The results are summarized in the following table:

TABLE 5-1
Anticoagulant effect of polymer-heparin conjugates

| | IIa | Xa |
|---|---|---|
| NaOAc | 0.145 | 0.191 |
| F32 | 0.121333333 | 0.208 |
| 2 ug Hep | 0.071833333 | 0.131866667 |
| 20 ug Hep | 0.070533333 | 0.085083333 |
| 2 ug Hep + F32 | 0.0805 | 0.199166667 |
| 20 ug Hep + F32 | 0.256533333 | 0.1648 |

### Example 6

### Heparin and Internalized Heparin: Dual Mechanisms to Inhibit Prostate Cancer Growth

Fibroblast growth factor (FGF) family members play an important role in the growth and progression of prostate cancer. The activity of FGFs is modulated by heparin/heparan sulfate-like glycosaminoglycans (HSGAGs), which interact with FGFs as well as their cell surface tyrosine kinase receptors. The ability of HSGAG to regulate prostate cancer growth was investigated. Heparin was found to prevent PC-3 cell growth. This growth inhibition was attributed to heparin preventing FGF2-mediated proliferation. PC-3 tumor growth was also inhibited by heparin in vivo. The ability of heparin complexed to poly(β-amino ester)s (PAEs), which promote endocytosis preferentially into cancer cells, was additionally examined. Internalized heparin inhibited PC-3 growth more efficaciously than heparin alone in vitro. In vivo, internalized heparin reduced PC-3 tumor growth. Heparin alone, but not internalized heparin, had an in vivo anticoagulant effect. Each of heparin and internalized heparin inhibited prostate cancer growth. Internalized heparin, however, more efficaciously inhibits primary tumor growth and prevents secondary effects associated with heparin.

In American men, prostate cancer is the most common cancer and second leading cause of cancer death (1). The growth and progression of prostate cancers has been associated with the activities of fibroblast growth factor (FGF) and the FGF receptor (FGFR). FGF1 (2), FGF2 (3), FGF6 (4), FGF8 (5), and FGF9 (6), for example, have each been demonstrated to be produced by and to regulate the activity of prostate cancer cells. The corresponding FGFRs that can support signal transduction downstream of the various FGF are also expressed by prostate cancer cells (6-9). The presence of FGFs and FGFRs provides the basis for an autocrine loop by which FGF-FGFR activity is thought to enhance prostate cancer cell proliferation (7).

FGF2 and its receptor, FGFR1, have emerged as critical regulators of prostate cancer as well as benign prostatic hypertrophy (3, 6, 9-11). FGFs interact not only with cell surface tyrosine kinase FGFRs, but also the heparin/heparan sulfate-like glycosaminoglycan (HSGAG) component of heparan sulfate (HS) proteoglycans (12-14). HSGAGs interact with both the ligand and the receptor, promoting ligand and subsequent receptor oligomerization. FGFR dimerization subsequently leads to tyrosine kinase phosphorylation and signal transduction (15-18). HSGAGs would be expected to modulate prostate cancer given the importance of FGFs and FGFRs.

Cancer growth, progression, and mortality can all be influenced by HSGAGs (19, 20). The 48 disaccharide building blocks that compose the 10-100-mer HSGAG biopolymer allow HSGAGs to regulate a wide variety of important processes involved with cancer, including growth factor activity and angiogenesis (19, 21, 22). When in the extracellular matrix (ECM), HSGAGs can bind growth factors and angiogenesis promoters, preventing their activity (23). Heparin, a highly sulfated HSGAG, can reduce the mortality associated with cancer by preventing fatal pulmonary embolisms secondary to deep venous thrombosis (20, 24). Nonetheless, the potential therapeutic use of HSGAGs in prostate cancer has not been well defined.

In this study, how HSGAGs influenced PC-3 growth, both in vitro and in vivo was examined. Heparin was found to successfully inhibit cell growth by preventing FGF2-mediated proliferation. Sufficiently high doses of heparin also inhibited tumor growth in vivo. Additionally, whether controlled internalization of heparin by complexation with poly(β-amino ester)s (PAEs), which targets cancer cells based on their increased endocytic rate and induces apoptotic cell death, could also prevent PC-3 growth was also examined. Internalized heparin more effectively inhibited PC-3 growth in vitro than heparin, and was not permissive to in vivo tumor growth. Heparin can therefore be used in multiple ways to prevent prostate cancer growth.

Materials and Methods

Proteins and Reagents

Fetal bovine serum (FBS) was from Hyclone (Logan, Utah). L-glutamine, penicillin/streptomycin, phosphate buffered saline (PBS), and Trizol reagent were obtained from GibcoBRL (Gaithersburg, Md.). Porcine intestinal mucosa heparin was from Celsus Laboratories (Columbus, Ohio). Recombinant human FGF2 was a gift from Scios, Inc. (Mountainview, Calif.). Recombinant heparinases were produced as described (26). Kinase inhibitors LY294002, PD98059, SB203580, and U0126 were from Promega (Madison, Wis.).

Cell Culture

PC-3 cells (American Type Culture Collection, Manassas, Va.) were maintained in Ham's F12K medium (American Type Culture Collection) supplemented with 1.5 mg/mL sodium bicarbonate, 100 μg/ml penicillin, 100 U/ml streptomycin, 500 μg/ml L-glutamine and 10% FBS. Cells were grown in 75 cm² flasks at 37° C. in a 5% CO₂ humidified incubator. Confluent cultures were split 1:3 to 1:6, two to three times per week.

Proliferation Assays

PC-3 cells were grown until confluence in 75 cm² flasks. Each flask was washed with 20 ml PBS, and treated with 3 ml trypsin-EDTA at 37° C. for 3-5 minutes, until cells detached. Cells were centrifuged for 3 minutes at 195×g, the supernatant was aspirated, and the cells were resuspended in 10 ml media. The cell suspension was diluted to 50,000 cells/ml based on the readings of an electronic cell counter. The suspension was plated 1 ml/well into 24-well tissue culture plates. After a 24 hour incubation in a 5% CO₂, 37° C. humidified incubator, the cells were washed with serum free media, supplemented with media containing 0.1% FBS, and incubated for 24 hours. Cells were treated with heparin, HS or FGF2 as appropriate. Heparin was added at 500 ng/ml unless otherwise noted. FGF2 was added at 100 ng/ml unless otherwise specified. Cells were then incubated for 72 hours. Wells were then washed twice with PBS and treated with 0.5 ml trypsin-EDTA/well and incubated for 10 minutes at 37° C. Whole cell number was determined using an electronic cell counter. Data were averaged over three experiments, each consisting of four wells per condition.

For antibody and kinase inhibitor experiments, antibodies and kinase inhibitors were added prior to HSGAGs or FGF2. Antibodies to FGF2, FGFR1, or FGFR3 were added to yield a final dilution of 1:100. Kinase inhibitors were added sufficient to yield final concentrations of 50 μM LY294002, 20 μM PD98059, 20 μM U0126, and 1 μM SB203580.

To produce heparin digests, heparin was treated with PBS, heparinase I (hepI), or hepIII for 30 minutes, and boiled for 30 prior to addition to cells. Digestion was verified and quantified by UV spectroscopy at 232 nm (27). Digests were added to yield a final HSGAG concentration of 500 ng/ml.

Polymer-Heparin Conjugates

Nine polymers (C32, D94, E28, F28, F32, U28, U32, JJ28, and JJ32) were selected from a library of 2350 PAEs, as they enabled highly efficient DNA transfection (28, 29). Polymers were prepared as described (28). To form conjugates, PAEs at 100 mg/ml in dimethyl sulfoxide were added to heparin in 25 mM NaOAc as appropriate to yield the desired PAE:heparin (w/w) ratio. The mixture was shaken gently at room temperature for five minutes, and diluted in PBS as appropriate for subsequent assays. Conjugates were used immediately after synthesis

A preliminary screen was performed on PC-3 cells by proliferation assay using the nine polymers described at polymer:heparin (w/w) ratios of 10:1, 20:1, 30:1, 40:1, and 60:1. The three best formulations (polymer and ratio) were selected and analyzed further. From this, a single best polymer was selected for subsequent use. In vitro assessment of polymer activity was measured by proliferation assay with a heparin concentration of 1 μg/ml. In vivo assessment was performed by intratumoral injection.

RT-PCR

A quantity of 5 μg of total RNA was isolated from PC-3 cells using Trizol reagent (Life Tech, Rockville, Md.), and reverse transcription was performed with random hexamers. Specific oligomers were designed based on the published sequences of FGFR isoforms in order to detect their expression. Sequences of primer pairs corresponding to distinct FGFR isoforms were as follows: FGFR1b: 5′-TGG AGC AAG TGC CTC CTC-3′ (SEQ ID NO:1) and 5′-ATA TTA CCA CTT CGA TTG GTC-3′ (SEQ ID NO:2); FGFR1c: 5′-TGG AGC TGG AAG TGC CTC CTC-3′ (SEQ ID NO:3) and 5′-GTG ATG GGA GAG TCC GAT AGA-3′ (SEQ ID NO:4); FGFR2b: 5′-GTC AGC TGG GGT CGT TTC ATC-3′ (SEQ ID NO:5) and 5′-CTG GTT GGC CTG CCC TAT ATA-3′ (SEQ ID NO:6); FGFR2c: 5′-GTC AGC TGG GGT CGT TTC ATC-3′ (SEQ ID NO:7) and 5′-GTG AAA GGA TAT CCC AAT AGA-3′ (SEQ ID NO:8); FGFR3b: 5′ GTA GTC CCG GCC TGC GTG CTA-3′ (SEQ ID NO:9) and 5′-GAC CGG TTA CAC AGC CTC GCC-3′ (SEQ ID NO:10); FGFR3c: 5′-GTA GTC CCG GCC TGC GTG CTA-3′ (SEQ ID NO:11) and 5′-TCC TTG CAC AAT GTC ACC TTT-3′ (SEQ ID NO:12); and FGFR4: 5′-CCC TGC CGG GAT CGT GAC CCG-3′ (SEQ ID NO:13) and 5′-TCG AAG CCG CGG.CTG CCA AAG-3′ (SEQ ID NO: 14). To control for total cell protein, RT-PCR was also performed on β-actin using the primers 5′-GCC AGC TCA CCA TGG ATG ATG ATA T-3′ (SEQ ID NO:15) and 5′-GCT TGC TGA TCC ACA TCT GCT GGA A-3′ (SEQ ID NO:16). PCR was performed using the Advantage-GC cDNA kit from Clontech as per manufacturer's instructions (Palo Alto, Calif.). Prior to experimental use, primers were confirmed to detect and have specificity towards given FGFR isoforms using BaF3 cells transfected with various FGFRs (27, 30).

Measurement of Anti-Coagulant Activity

In vitro anti-Xa and anti-IIa experiments were performed as described (31-34). The anti-Xa assay was performed by using S-2222 as the chromogenic substrate. The anti-IIa assay was performed by using S-2238 as the chromogenic substrate.

For in vivo assessment of Factor Xa and Factor IIa activity, mice were treated with heparin or F32-heparin and sacrificed by CO₂ asphyxiation within 24 hours. Cardiac puncture was used to collect ˜500 μl blood per animal. For coagulation studies, blood was centrifuged the plasma was extracted, and the activities of plasma Factors Xa and IIa were measured. Plasma was diluted 1:150 in PBS to a final volume of 90 μl, and treated with 600 ng of chromogenic substrate for Factor Xa or for Factor IIa (Sigma, St. Louis Mo.) as appropriate in 10 μl PBS. Change in absorbance per second was measured at 405 nm.

In Vivo Tumor Growth Assays

Xenografts were generated in nude (nu/nu.c) Harlan (Indianapolis, Ind.) Sprague-Dawley rats via the subcutaneous injection of 5×10⁶ PC-3 human prostatic adenocarcinoma cells into each flank. Tumors were allowed to grow for 1 week until tumor volumes were approximately 50 mm³, and intratumoral injections were initiated (day 0). Only mice in which tumors on both sides were of similar size were used for the remainder of the experiment. Heparin was prepared in 2.5 mM sodium acetate in PBS, in a final volume of 100 μl. F32-heparin conjugates were produced as described at a 10:1 polymer:heparin (w/w) ratio, and diluted in PBS.

Three dosing regimens were employed. At least six mice were used for a given experimental point, predicted to yield p<0.05 with power=80%. First, heparin alone at various concentrations (5 ng-50 μg) was injected into six mice per dose on day 0 and each subsequent day through the experimental end point (day 8). An equivalent volume of vehicle (referred to as NaOAc) alone was injected into five mice. Second, six mice per dose were treated on day 0 with vehicle or heparin (500 ng to 400 μg), and tumor size was measured over eight days. Finally, 10 mice per dose were treated once with vehicle, heparin (5 μg-400 μg), or the equivalent amounts of heparin conjugated to F32 at a 10:1 polymer:heparin (w/w) ratio. Tumors were measured by caliper throughout the experiment, and volume was calculated as length×width×height×π/6. Liver function tests and complete blood counts were performed on all treated animals using blood collected via cardiac puncture.

Results and Discussion

Heparin Inhibits PC-3 Proliferation by Preventing FGF2-Mediated Growth

Human prostate cancer cells express FGFs as well as the appropriate FGFR isoforms to enable a cellular mediated response both in vitro and in vivo (2-6, 10, 35-37). Autocrine FGF activity through cell surface FGFRs is common in human prostate cancer. Prostate cancer cells additionally switch their FGF and FGFR expression with invasion and malignancy (38). FGF is thought to enhance prostate cancer cell proliferation.

PC-3 cells are androgen-insensitive and highly metastatic human prostate cancer cells, whose survival can be increased by FGF2 (9, 39). FGF2 and FGFR1 are critical regulators of prostate cancer tumorigenicity (8). HSGAGs are known to alter the growth and progression of cancers through a variety of mechanisms including via FGF2 (40). Although HSGAGs serve to enhance the activity of FGF2 by promoting ligand dimerization, ternary complex formation, and downstream signal transduction (15), extracellular heparin can serve as a biological “sink,” binding FGFs and preventing cellular mediated responses (23).

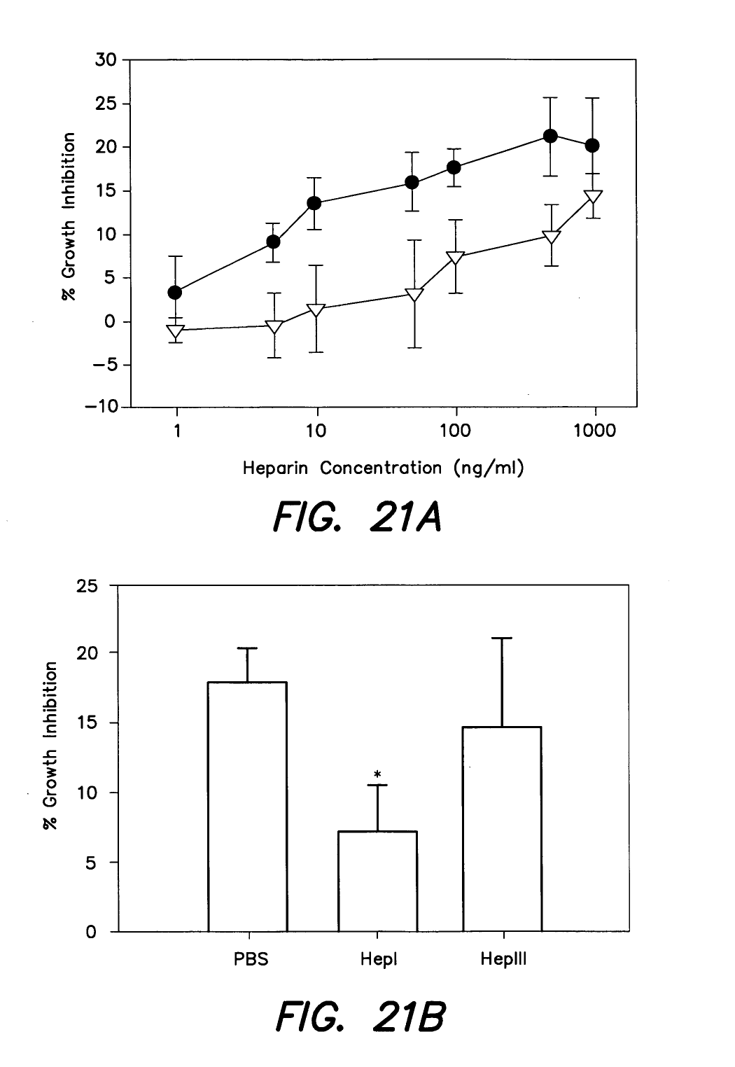

To investigate whether HSGAGs could be used to inhibit human prostate cancer cell growth, PC-3 cells were treated with heparin, and the effect on proliferation was determined. Heparin reduced PC-3 whole cell number in a dose-dependent manner (FIG. 21A), with a maximal effect of 21.0±4.4% at 500 ng/ml. HS also reduced whole cell number in a dose-dependent manner. A maximal response of a 14.3±2.6% reduction in whole cell number was observed at 1 μg/ml, the maximal concentration tested. Nonetheless, heparin elicited a more potent response than HS. To confirm the ability of HSGAGs to inhibit PC-3 growth, heparin was pretreated with PBS, hepI, or hepIII. Partial digestion was confirmed and quantified by UV spectroscopy at 232 nm (27). PBS-treated heparin reduced whole cell number 17.8±2.5% (FIG. 21B), not significantly different from the cellular response elicited with hepIII digested heparin (p>0.45). HepI treated heparin only reduced whole cell number by 7.0±3.5%, significantly less than PBS-treated heparin (p<0.006). Highly sulfated HSGAGs therefore elicit the greatest growth inhibitory response from PC-3 cells.

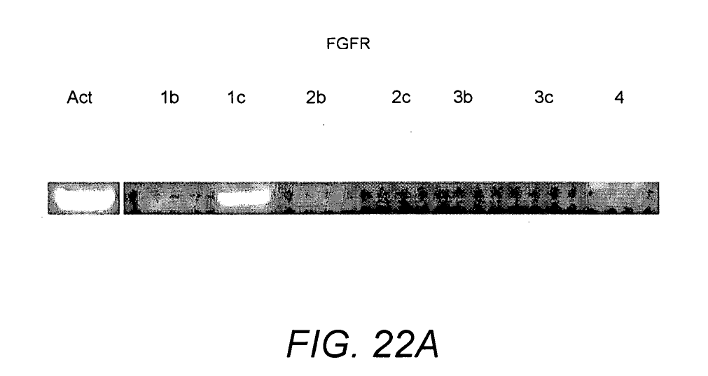

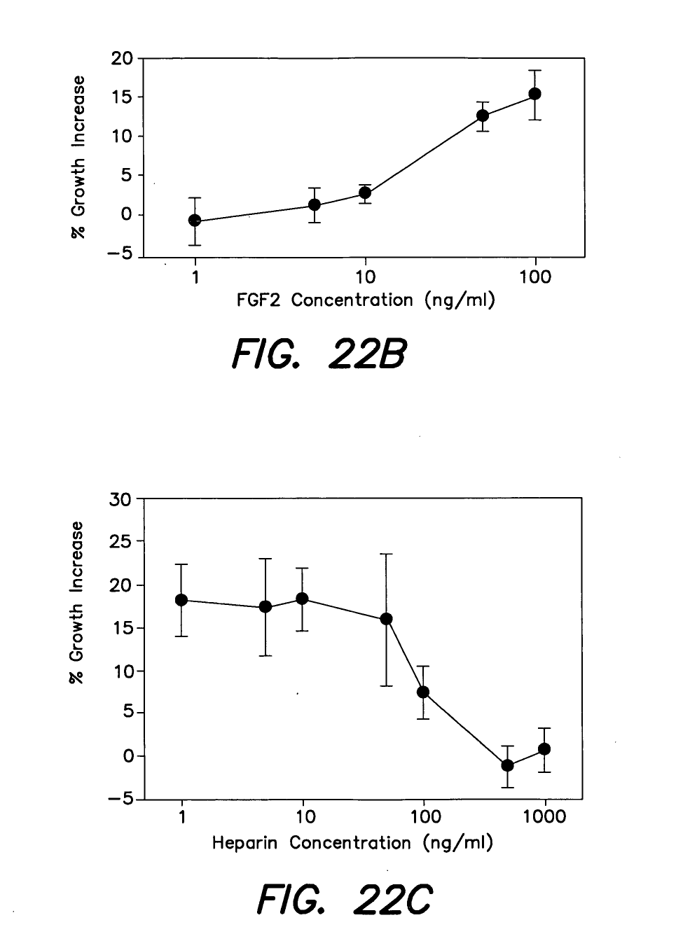

How heparin elicited its growth inhibitory effects was then examined. PC-3 cells produce FGF2, 80-90% of which remains in the cytoplasm while the other 10-20% is secreted into the ECM (36). Heparin can inhibit the activity of angiogenic factors by preventing their interaction with cell surface HSGAGs (23). To investigate whether heparin reduced whole cell number by inhibiting FGF2 activity, it was verified that PC-3 cells could respond to FGF2. RT-PCR demonstrated that PC-3 cells predominantly expressed FGFR1c (FIG. 22A), which supports the activity of FGF2 (14, 30). The addition of FGF2 induced the proliferation of PC-3 cells, with a maximal effect of 15.6±3.1% observed with 100 ng/ml FGF2 (FIG. 22B). To investigate whether heparin could reduce whole cell number by preventing FGF2 activity, PC-3 cells were treated with 100 ng/ml FGF2 and varying concentrations of heparin (FIG. 22C). Heparin concentrations of 50 ng/ml and less permitted FGF2-mediated proliferation. At 100 ng/ml heparin, however, the increase in whole cell number was reduced to 7.7±3.0%, and at 500 ng/ml heparin, the cells responded as if no FGF2 had been added (−0.9%±2.4%). Heparin can, therefore, prevent FGF2-mediated cell growth.

To confirm that heparin inhibited proliferation by preventing FGF2 activity, it was next examined whether other techniques to block FGF2 and its downstream signaling would similarly reduce whole cell number. Correspondingly, treating PC-3 cells with antibodies to FGF2 (56.6±2.2%; p<1×10-¹⁰) or to FGFR1 (58.2±1.8 p<3×10⁻¹²) reduced whole cell number. Furthermore, the addition of heparin failed to reduce whole cell number when cells were pretreated with antibodies to FGF2 (−5.0±6.0; p>0.14) or FGFR1 (0.0±4.5%; p>0.99). Antibodies to FGFR3 did not prevent heparin from reducing whole cell number (p<2×10⁻⁶). The specificity of the various antibodies was confirmed by performing proliferation assays with BaF3 cells transfected with specific FGFRs (30).

Inhibition of processes downstream of FGF2, with LY294002, PD98059, or U0126, similarly prevented heparin-mediated growth inhibition. LY294002 inhibits phosphoinositol 3-kinase, which is downstream of FGFRs (42). PD98059 and U0126 inhibit Erk/Mek and Mek respectively, which are associated with the proliferative activities of FGF2 through FGFR1 (43). The use of kinase inhibitors such as SB203580, which are not downstream of FGF2, however, had no effect. These findings provide additional evidence that heparin prevents FGF2 activity, thereby inhibiting PC-3 proliferation.

Heparin Inhibits PC-3 Cell Growth in Vivo

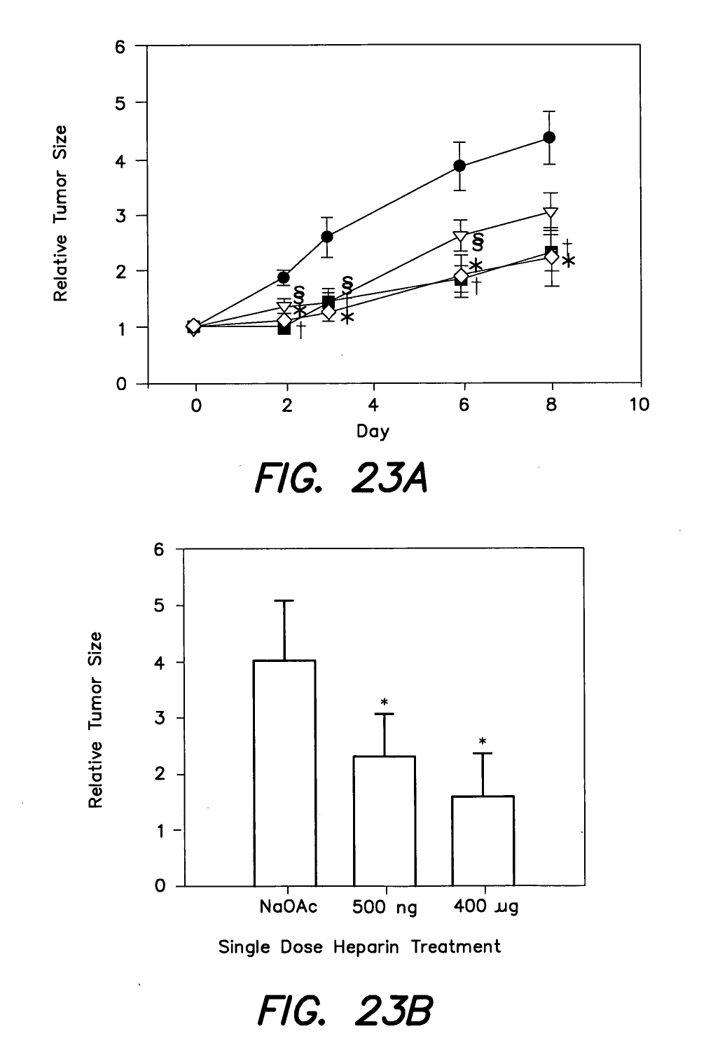

Given the ability of heparin to inhibit PC-3 cell growth in vitro, its effects in vivo were examined. PC-3 tumors were formed in the flanks of mice, allowed to grow, and heparin was injected intratumorally, either each experimental day or only once. Tumors were first injected with heparin each day ranging between 5 ng and 50 μg per injection, for eight days. Heparin injections inhibited tumor growth compared to the vehicle (NaOAc) control (FIG. 23A). Increasing amounts of heparin progressively increased the magnitude of the growth inhibitory effect of heparin up to 500 ng. Injections of greater amounts of heparin, however, did not inhibit PC-3 tumor growth to a greater extent.

The effects of single dose heparin was then determined (FIG. 23B). PC-3 tumors were injected with heparin between 500 ng and 400 μg (8×50 μg). Treatment with 500 ng heparin significantly reduced tumor growth from 4.0±1.1-times the day 0 tumor (with NaOAc) to 2.3±0.8-times the day 0 tumor (with heparin treatment; p<0.05). Increasing doses inhibited tumor growth to a greater extent, with the most efficacious response observed with 400 μg, where final tumor volume was 1.6±0.8-times the size of the day 0 tumor, ˜61% (p<0.02) smaller than the NaOAc treated tumor. The highest dose of heparin therefore prevented tumor growth. No other single dose or repeated dose that was examined elicited this effect.

These results demonstrate that heparin effectively inhibits tumor growth. The importance of FGF-FGFR signaling has been well supported in cancer cell lines, in animal models, and in human tissues. The in vitro results demonstrate that heparin does inhibit FGF2 signaling and the same mechanism may enable in vivo prostate cancer growth inhibition. This mechanism was not confirmed in the in vivo experiments performed, but evidence of this possibility has been shown (7, 11, 23). Small molecule inhibitors of FGFR signaling have additionally shown preliminary success as a potential cancer therapy in clinical trials (44). Especially as FGF2 release may be associated with more aggressive prostate cancers (36), the results presented suggest that heparin treatment may serve as a therapeutic in cancer, such as prostate cancer, both by preventing tumor growth, and by preventing coagulation-related complications associated with cancer (20, 24).

Internalized Heparin Induces PC-3 Cell Death

Heparin itself has been demonstrated to have a wide range of potential roles in cancer growth and progression (45). The data presented suggest potential therapeutic value in cancer, such as prostate cancer, by inhibiting essential autocrine factors. The polydispersity of HSGAGs leads to a low percentage of sequences that regulate a given process and therefore, an increased potential for secondary, and possibly undesirable, activities (46). The use of a delivery vehicle to target the activities of heparin could minimize the potential for side effects and therefore promote therapeutic use for cancer.

PAEs are a class of polymers that has been demonstrated to efficiently bind DNA and promote its internalization into cells (28, 29, 47, 48). PAEs condense DNA through electrostatic interactions between the cationic polymers and the anionic DNA. PAE-DNA complexes that are best internalized by cells have the most positive zeta potentials (49). Although heparin is more anionic than DNA, PAEs can also condense heparin. Conjugates formed between specific PAEs and heparin yield positively charged complexes that enable endocytosis, preferentially into cancer cells. The selectivity of PAE-heparin conjugates for cancer cells is based on their increased rate of endocytosis relative to non-transformed cells, which is associated with the upregulation of factors found in epithelial tumors including those of the prostate and colon (50, 51). Therefore, it was investigated whether PAE-heparin conjugates would offer a more efficacious and potentially safer method to target cancer cells with heparin.

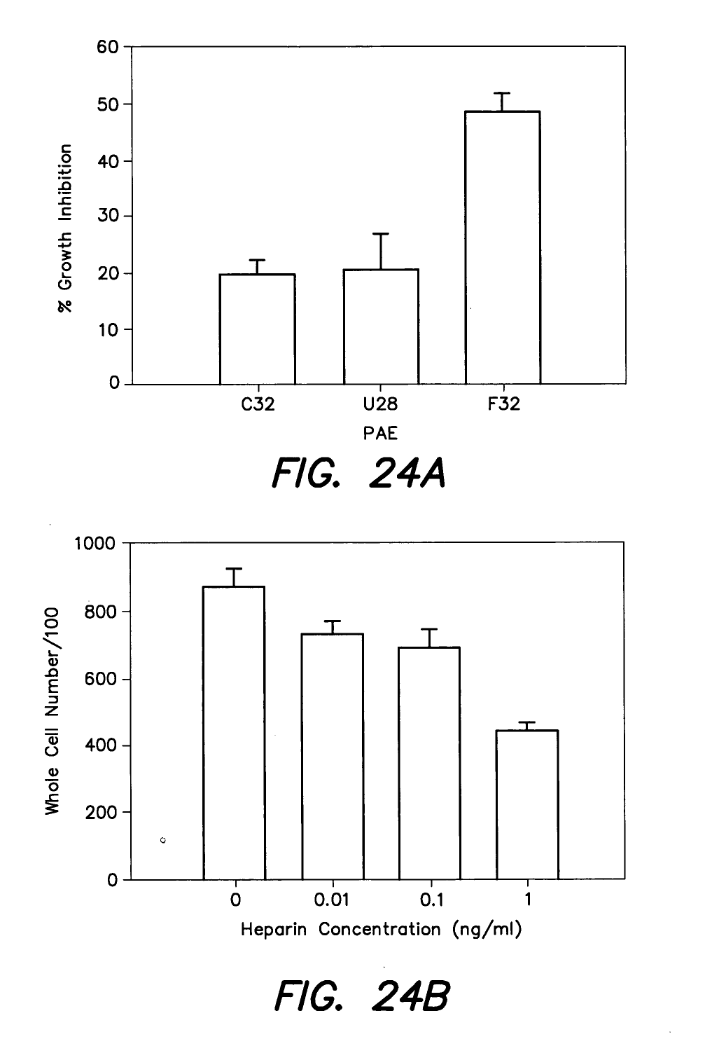

Previous studies with PAE-heparin conjugates focused on selected members of a 140-member polymer library (47). As positive zeta potentials correlated to internalization efficiency, polymers from a given PAE library that best enabled DNA transfection also supported heparin internalization. A subsequent library of 2350 polymers was constructed by the combinatorial addition of 94 amines and 25 diacrylates (48). Nine polymers (C32, D94, E28, F28, F32, U28, U32, JJ28, and JJ32) selected from previous screens to have the best in vitro transfection rates (28, 29) were used to examine the effects of internalized heparin on PC-3 cells at polymer:heparin (w/w) ratios of 10:1, 20:1, 30:1, 40:1, and 60:1, all using a final heparin concentration of 1 μg/ml. The results of this screen identified three polymers that produced the greatest reduction in whole cell number: C32 (60:1), U28 (60:1), and F32 (10:1). At the concentrations examined, polymer alone did not affect whole cell number. The ability of these polymers to internalize heparin was subsequently verified by fluorescent microscopy using fluorescein-conjugated heparin. These three polymers were tested specifically to validate the growth inhibition observed on the first screen (FIG. 24A). C32 (19.4±2.5%; p<6×10⁻⁵), U28 (20.1±6.6%; p<0.008) and F32 (48.4±3.2; p<6×10⁻⁶) again showed substantial growth inhibition, with the greatest effects observed with F32.

A dose-response curve was produced using F32, which demonstrated that the ˜50% growth inhibition observed could not be elicited by heparin concentrations less than 1 μg/ml (FIG. 24B). Furthermore, F32 alone at 10 μg/ml did not alter whole cell counts. PC-3 cells were then treated with polymer-heparin conjugates for two hours, washed, and incubated for three days in unsupplemented media to determine if increases in magnitudes of response were related to more rapid internalization. C32 and U28 had no effect, while F32 treatment for two hours reduced whole cell number by 10.0±0.8% (p<0.02).

Internalized Heparin Prevents PC-3 Tumor Growth

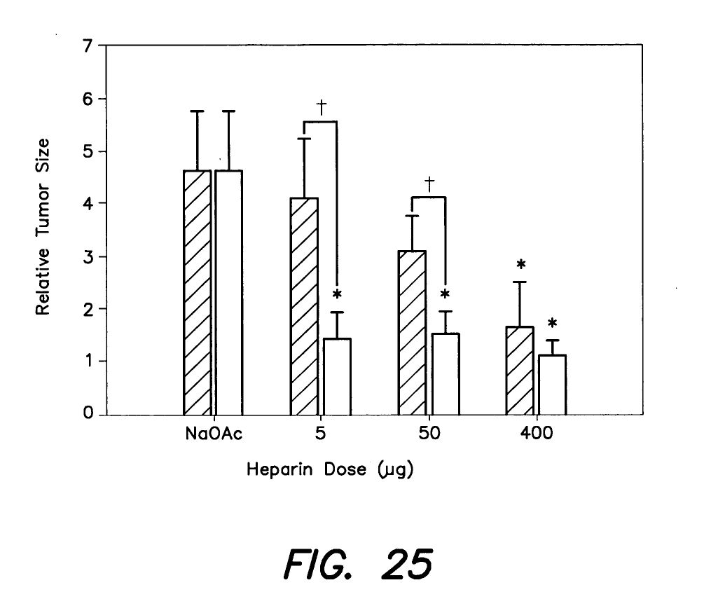

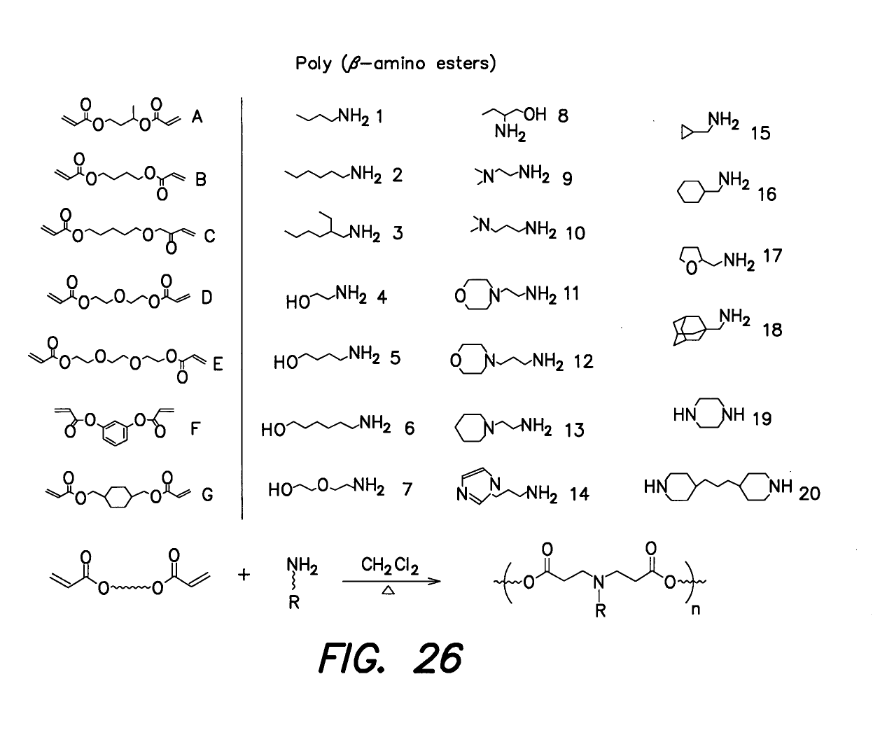

F32-heparin conjugates inhibited PC-3 cell growth not only better than the other polymer-heparin conjugates examined, but also more effectively than heparin alone. Therefore, the effects of F32-heparin conjugates in vivo were examined. PC-3 tumors were treated once with heparin (5 μg to 400 μg), F32-heparin (10:1, w/w polymer:heparin, 5 μg to 400 μg heparin), or NaOAc, and tumor volume was measured over 8 days. Liver function tests and complete blood counts were performed to identify any systemic toxicity associated with heparin or F32-heparin. No measure was significantly different than that observed with NaOAc treated rats. Heparin alone inhibited tumor growth in a dose-dependent manner, with the highest dose (1.7±0.9-times the original tumor volume) preventing significant tumor growth (FIG. 25). F32-heparin conjugates effectively prevented tumor growth at each of 5 μg (1.5±0.4-times the original tumor volume), 50 μg (1.4±0.5-times the original tumor volume), and 400 μg (1.1±0.3-times the original tumor volume). Polymer alone had no significant effect on tumor size (p>0.63). F32-heparin inhibited tumor growth significantly more than heparin alone at doses of 5 μg (p<0.005) and 50 μg (p<0.004). At 50 μg and 400 μg, tumors did not grow.

The anticoagulant effects of heparin and F32-heparin were additionally examined. In addition to other mechanisms, heparin is known to reduce cancer-associated mortality through anticoagulant effects (20, 24). Anti-Xa and anti-IIa activities were first measured in vitro. Heparin produced a Xa/IIa ratio of 1.3, consistent with previous findings (52). Neither Xa nor IIa activity was detectable, however, with F32-heparin. The anticoagulant effects of heparin and F32-heparin were next examined in vivo. Serum was then collected from animals treated with heparin and F32-heparin, and the anticoagulant effects were determined. All doses of heparin had significant anticoagulant effects, while F32-heparin demonstrated no change in the anticoagulant profile of treated mice. The coagulation assays additionally suggest that F32-heparin elicited the increased magnitude of response by heparin internalization rather than by slow-release. Should F32-heparin conjugates act through slow release of heparin, an anticoagulant effect would have been expected, albeit potentially less than that elicited by heparin alone. The absence of any detectable anticoagulant effect is not consistent with a slow-release mechanism. Furthermore, single dose heparin yielded a greater magnitude of response than repeated doses. F32-heparin therefore increases the magnitude of growth inhibition in a slow release-independent manner, consistent with the heparin internalization mechanism.

Internalized heparin may therefore be an effective way to prevent prostate cancer growth, both in vitro and in vivo, and thus is a potential cancer therapeutic for prostate cancer as well as other cancers. The ability of internalized heparin to inhibit prostate cancer growth, better than heparin alone, validates the use of endocytic rate as a mechanism by which cancer cells can be targeted. Additionally, no side effects were detected by liver function tests, complete blood counts or coagulation assays. PAE-heparin conjugates therefore have increased anti-cancer activity in vivo with reduced or no apparent side effects.

The data presented demonstrate that heparin can be harnessed to inhibit cancer growth by multiple mechanisms. Heparin alone can prevent the activity of angiogenic and tumor growth promoting factors such as FGF2 (23), and therefore inhibit PC-3 growth in vitro and in vivo, while also exhibiting anticoagulant effects. As a result, heparin alone would serve as an important secondary anti-cancer agent by reducing tumor growth as well as potential coagulation-related mortality events (20, 24). Conjugating heparin to PAEs can promote more potent growth inhibition without anticoagulant behavior. PAE-heparin conjugates could thus better function as a primary anti-cancer agent. Tailoring the delivery mechanism can therefore change the anti-cancer behavior of heparin, an effect that can potentially be harnessed to achieve a desired subset of therapeutic behaviors.

### REFERENCES FOR EXAMPLE 6

1. Jemal, A., Murray, T., Ward, E., Samuels, A., Tiwari, R. C., Ghafoor, A., Feuer, E. J., and Thun, M. J. Cancer statistics, 2005. CA Cancer J Clin, 55: 10-30, 2005.
2. Dorkin, T. J., Robinson, M. C., Marsh, C., Neal, D. E., and Leung, H. Y. aFGF immunoreactivity in prostate cancer and its co-localization with bFGF and FGF8. J Pathol, 189: 564-569, 1999.
3. Giri, D., Ropiquet, F., and Ittmann, M. Alterations in expression of basic fibroblast growth factor (FGF) 2 and its receptor FGFR-1 in human prostate cancer. Clin Cancer Res, 5: 1063-1071, 1999.
4. Ropiquet, F., Giri, D., Kwabi-Addo, B., Mansukhani, A., and Ittmann, M. Increased expression of fibroblast growth factor 6 in human prostatic intraepithelial neoplasia and prostate cancer. Cancer Res, 60: 4245-4250, 2000.
5. Dorkin, T. J., Robinson, M. C., Marsh, C., Bjartell, A., Neal, D. E., and Leung, H. Y. FGF8 over-expression in prostate cancer is associated with decreased patient survival and persists in androgen independent disease. Oncogene, 18: 2755-2761, 1999.
6. Giri, D., Ropiquet, F., and Ittmann, M. FGF9 is an autocrine and paracrine prostatic growth factor expressed by prostatic stromal cells. J Cell Physiol, 180: 53-60, 1999.
7. Ozen, M., Giri, D., Ropiquet, F., Mansukhani, A., and Ittmann, M. Role of fibroblast growth factor receptor signaling in prostate cancer cell survival. J Natl Cancer Inst, 93: 1783-1790, 2001.
8. Feng, S., Wang, F., Matsubara, A., Kan, M., and McKeehan, W. L. Fibroblast growth factor receptor 2 limits and receptor 1 accelerates tumorigenicity of prostate epithelial cells. Cancer Res, 57: 5369-5378, 1997.
9. Rosini, P., Bonaccorsi, L., Baldi, E., Chiasserini, C., Forti, G., De Chiara, G., Lucibello, M., Mongiat, M., Iozzo, R. V., Garaci, E., Cozzolino, F., and Torcia, M. G. Androgen receptor expression induces FGF2, FGF-binding protein production, and FGF2 release in prostate carcinoma cells: role of FGF2 in growth, survival, and androgen receptor down-modulation. Prostate, 53: 310-321, 2002.
10. Ropiquet, F., Giri, D., Lamb, D. J., and Ittmann, M. FGF7 and FGF2 are increased in benign prostatic hyperplasia and are associated with increased proliferation. J Urol, 162: 595-599, 1999.
11. Polnaszek, N., Kwabi-Addo, B., Peterson, L. E., Ozen, M., Greenberg, N. M., Ortega, S., Basilico, C., and Ittmann, M. Fibroblast growth factor 2 promotes tumor progression in an autochthonous mouse model of prostate cancer. Cancer Res, 63: 5754-5760, 2003.
12. Givol, D. and Yayon, A. Complexity of FGF receptors: genetic basis for structural diversity and functional specificity. Faseb J, 6: 3362-3369, 1992.
13. Rapraeger, A. C. The coordinated regulation of heparan sulfate, syndecans and cell behavior. Curr Opin Cell Biol, 5: 844-853, 1993.
14. Ornitz, D. M., Xu, J., Colvin, J. S., McEwen, D. G., MacArthur, C. A., Coulier, F., Gao, G., and Goldfarb, M. Receptor specificity of the fibroblast growth factor family. J Biol Chem, 271: 15292-15297, 1996.
15. Yayon, A., Klagsbrun, M., Esko, J. D., Leder, P., and Ornitz, D. M. Cell surface, heparin-like molecules are required for binding of basic fibroblast growth factor to its high affinity receptor. Cell, 64: 841-848, 1991.
16. Guimond, S., Maccarana, M., Olwin, B. B., Lindahl, U., and Rapraeger, A. C. Activating and inhibitory heparin sequences for FGF-2 (basic FGF). Distinct requirements for FGF-1, FGF-2 and FGF-4. J Biol Chem, 268: 23906-23914, 1993.
17. Kan, M., Wu, X., Wang, F., and McKeehan, W. L. Specificity for fibroblast growth factors determined by heparan sulfate in a binary complex with the receptor kinase. J Biol Chem, 274: 15947-15952, 1999.
18. Pye, D. A., Vives, R. R., Hyde, P., and Gallagher, J. T. Regulation of FGF-1 mitogenic activity by heparan sulfate oligosaccharides is dependent on specific structureal features: differential requirements for the modulation of FGF-1 and FGF-2. Glycobiology, 10: 1183-1192, 2000.
19. Iozzo, R. V. and San Antonio, J. D. Heparan sulfate proteoglycans: heavy hitters in the angiogenesis arena. Journal of Clinical Investigation, 108: 349-355, 2001.
20. Zacharski, L. R. and Ornstein, D. L. Heparin and cancer. Thromb Haemost, 80: 10-23, 1998.
21. Folkman, J. Angiogenesis-dependent diseases. Semin Oncol, 28: 536-542, 2001.
22. Perrimon, N. and Bernfield, M. Specificities of heparan sulphate proteoglycans in developmental processes. Nature, 404: 725-728, 2000.
23. Fannon, M., Forsten-Williams, K., Dowd, C. J., Freedman, D. A., Folkman, J., and Nugent, M. A. Binding inhibition of angiogenic factors by heparan sulfate proteoglycans in aqueous humor: potential mechanism for maintenance of an avascular environment. Faseb J, 17: 902-904, 2003.
24. Cosgrove, R. H., Zacharski, L. R., Racine, E., and Andersen, J. C. Improved cancer mortality with low-molecular-weight heparin treatment: a review of the evidence. Semin Thromb Hemost, 28: 79-87, 2002.
26. Natke, B., Venkataraman, G., Nugent, M. A., and Sasisekharan, R. Heparinase treatment of bovine smooth muscle cells inhibits fibroblast growth factor-2 binding to fibroblast growth factor receptor but not FGF-2 mediated cellular proliferation. Angiogenesis, 3: 249-257, 2000.
27. Berry, D., Shriver, Z., Natke, B., Kwan, C. P., Venkataraman, G., and Sasisekharan, R. Heparan sulphate glycosaminoglycans derived from endothelial cells and smooth muscle cells differentially modulate fibroblast growth factor-2 biological activity through fibroblast growth factor receptor-1. Biochem J, 373: 241-249, 2003.
28. Anderson, D. G., Peng, W., Akinc, A., Hossain, N., Kohn, A., Padera, R., Langer, R., and Sawicki, J. A. A polymer library approach to suicide gene therapy for cancer. Proc Natl Acad Sci USA, 101: 16028-16033, 2004.
29. Anderson, D. G., Akinc, A., Hossain, N., and Langer, R. Structure/property studies of polymeric gene delivery using a library of poly(beta-amino esters). Mol Therapy, In Press, 2005.
30. Berry, D., Kwan, C. P., Shriver, Z., Venkataraman, G., and Sasisekharan, R. Distinct heparan sulfate glycosaminoglycans are responsible for mediating Fibroblast Growth Factor-2 biological activity though different Fibroblast Growth Factor Receptors. Faseb J, 15: 1422-1424, 2001.
31. Teien, A. N. and Lie, M. Heparin assay in plasma: a comparison of five clotting methods. Thromb Res, 7: 777-788, 1975.
32. Teien, A. N. and Lie, M. Evaluation of an amidolytic heparin assay method: increased sensitivity by adding purified antithrombin III. Thromb Res, 10: 399-410, 1977.
33. Teien, A. N., Lie, M., and Abildgaard, U. Assay of heparin in plasma using a chromogenic substrate for activated factor X. Thromb Res, 8: 413-416, 1976.
34. Sundaram, M., Qi, Y., Shriver, Z., Liu, D., Zhao, G., Venkataraman, G., Langer, R., and Sasisekharan, R. Rational design of low-molecular weight heparins with improved in vivo activity. Proc Natl Acad Sci USA, 100: 651-656, 2003.
35. Leung, H. Y., Dickson, C., Robson, C. N., and Neal, D. E. Over-expression of fibroblast growth factor-8 in human prostate cancer. Oncogene, 12: 1833-1835, 1996.
36. Cronauer, M. V., Hittmair, A., Eder, I. E., Hobisch, A., Culig, Z., Ramoner, R., Zhang, J., Bartsch, G., Reissigl, A., Radmayr, C., Thurnher, M., and Klocker, H. Basic fibroblast growth factor levels in cancer cells and in sera of patients suffering from proliferative disorders of the prostate. Prostate, 31: 223-233, 1997.
37. Ropiquet, F., Giri, D., Kwabi-Addo, B., Schmidt, K., and Ittmann, M. FGF-10 is expressed at low levels in the human prostate. Prostate, 44: 334-338, 2000.
38. Yan, G., Fukabori, Y., McBride, G., Nikolaropolous, S., and McKeehan, W. L. Exon switching and activation of stromal and embryonic fibroblast growth factor (FGF)-FGF receptor genes in prostate epithelial cells accompany stromal independence and malignancy. Mol Cell Biol, 13: 4513-4522, 1993.
39. Nakamoto, T., Chang, C. S., Li, A. K., and Chodak, G. W. Basic fibroblast growth factor in human prostate cancer cells. Cancer Res, 52: 571-577, 1992.
40. Liu, D., Shriver, Z., Venkataraman, G., El Shabrawi, Y., and Sasisekharan, R. Tumor cell surface heparan sulfate as cryptic promoters or inhibitors of tumor growth and metastasis. Proc Natl Acad Sci USA, 99: 568-573, 2002.
42. Mochizuki, Y., Tsuda, S., Kanetake, H., and Kanda, S. Negative regulation of urokinase-type plasminogen activator production through FGF-2-mediated activation of phosphoinositide 3-kinase. Oncogene, 21: 7027-7033, 2002.
43. Chua, C. C., Rahimi, N., Forsten-Williams, K., and Nugent, M. A. Heparan sulfate proteoglycans function as receptors for fibroblast growth factor-2 activation of extracellular signal-regulated kinases 1 and 2. Circ Res, 94: 316-323, 2004.
44. Laird, A. D., Vajkoczy, P., Shawver, L. K., Thurnher, A., Liang, C., Mohammadi, M., Schlessinger, J., Ullrich, A., Hubbard, S. R., Blake, R. A., Fong, T. A., Strawn, L. M., Sun, L., Tang, C., Hawtin, R., Tang, F., Shenoy, N., Hirth, K. P., McMahon, G., and Cherrington SU6668 is a potent antiangiogenic and antitumor agent that induces regression of established tumors. Cancer Res, 60: 4152-4160, 2000.
45. Sasisekharan, R., Shriver, Z., Venkataraman, G., and Narayanasami, U. Roles of heparan-sulphate glycosaminoglycans in cancer. Nat Rev Cancer, 2: 521-528, 2002.
46. Walenga, J. M., Jeske, W. P., Bara, L., Samama, M. M., and Fareed, J. Biochemical and pharmacologic rationale for the development of a synthetic heparin pentasaccharide. Thromb Res, 86: 1-36, 1997.
47. Lynn, D. M., Anderson, D. G., Putnam, D., and Langer, R. Accelerated Discovery of Synthetic Transfection Vectors: Parallel Synthesis and Screening of a Degradable Polymer Library. J. Am. Chem. Soc., 123: 8155-8156, 2001.
48. Anderson, D. G., Lynn, D. M., and Langer, R. Semi-automated synthesis and screening of a large library of degradable cationic polymers for gene delivery. Angew Chem Int Ed, 42: 3153-3158, 2003.
49. Akinc, A., Lynn, D. M., Anderson, D. G., and Langer, R. Parallel synthesis and biophysical characterization of a degradable polymer library for gene delivery. J Am Chem Soc, 125: 5316-5323, 2003.
50. Rao, D. S., Hyun, T. S., Kumar, P. D., Mizukami, I. F., Rubin, M. A., Lucas, P. C., Sanda, M. G., and Ross, T. S. Huntingtin-interacting protein 1 is overexpressed in prostate and colon cancer and is critical for cellular survival. J Clin Invest, 110: 351-360, 2002.
51. Ross, T. S. and Gilliland, D. G. Transforming properties of the Huntingtin interacting protein 1/platelet-derived growth factor beta receptor fusion protein. J Biol Chem, 274: 22328-22336, 1999.
52. Morris, T. A., Marsh, J. J., Konopka, R., Pedersen, C. A., and Chiles, P. G. Anti-thrombotic efficacies of enoxaparin, dalteparin, and unfractionated heparin in venous thrombo-embolism. Thromb Res, 100: 185-194, 2000.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

We claim:

1. A composition, comprising:
a cationic polymer, and
a polysaccharide, wherein the polysaccharide is present in a intracellular therapeutically effective amount.

2. The composition of claim 1, wherein the polysaccharide is a glycosaminoglycan.

3. The composition of claim 2, wherein the glycosaminoglycan is a heparin/heparin sulfate-like glycosaminoglycan.

4. The composition of claim 2, wherein the glycosaminoglycan is heparin, heparan sulfate, enoxaparin, low molecular weight heparin (LMWH) or chondroitin sulfate.

5. The composition of claim 4, wherein the chondroitin sulfate is chondroitin sulfate A or chondroitin sulfate C.

6. The composition of claim 1, wherein the therapeutically effective amount is an amount effective to promote apoptosis.

7. The composition of claim 1, wherein the therapeutically effective amount is an amount effective to treat a disease characterized by abnormal cell proliferation.

8. The composition of claim 1, wherein the cationic polymer is degradable.

9. The composition of claim 1, wherein the cationic polymer is a poly(β-amino ester).

10. The composition of claim 9, wherein the poly(β-amino ester) is A5, A8, A11, B6, B9, B11, B14, C4, C12, C32, D6, D94, E7, E14, E28, F20, F28, G5, C32-2, U28, U28-3, JJ28-3, D94-5, E28-3, U32, U32-2, JJ28, JJ32, JJ32-3, F28-6, F32 or F32-2.

11.-31. (canceled)

32. A composition, comprising:
a polysaccharide,
a cationic polymer, and
a pharmaceutically acceptable carrier, wherein more cationic polymer is present in the composition (w/w) than the polysaccharide.

33.-41. (canceled)

42. A composition, comprising:
a poly(β-amino ester), and
a polysaccharide.

43.-50. (canceled)

51. A composition, comprising:
a cationic polymer,
a polysaccharide, and
a targeting molecule.

52.-59. (canceled)

60. A method for the intracellular delivery of a therapeutically effective amount of a polysaccharide, comprising:
administering the polysaccharide complexed to a cationic polymer to promote the uptake of the polysaccharide into a cell in a therapeutically effective amount.

61.-77. (canceled)

78. A method for promoting apoptosis in a subject, comprising:
administering a glycosaminoglycan intracellularly in an amount effective to promote apoptosis, wherein the glycosaminoglycan is delivered in an intracellular therapeutically effective amount to promote apoptosis.

79.-87. (canceled)

88. A method for treating a disease characterized by abnormal cell proliferation in a subject, comprising:
administering a glycosaminoglycan intracellularly in an amount effective to treat the disease, wherein the glycosaminoglycan is delivered in an intracellular therapeutically effective amount to treat the disease.

89.-100. (canceled)

101. A method for the intracellular delivery of a polysaccharide, comprising:
administering the composition of claim 42.

102. (canceled)

103. A method for the intracellular delivery of a polysaccharide, comprising:
administering the polysaccharide complexed to a cationic polymer to a non-macrophage cell, wherein the polysaccharide is not present in excess of the cationic polymer.

104. A method of promoting cell viability, comprising:
contacting a cell with a cationic polymer-polysaccharide conjugate prior to freezing the cell in an amount effective to increase the cell's viability when thawed.

105.-106. (canceled)

107. A method for inhibiting abnormal cell proliferation, comprising:
administering a glycosaminoglycan complexed to a cationic polymer in an amount effective to inhibit cell proliferation.

108.-120. (canceled)
